(19) United States
(12) Patent Application Publication
(10) Pub. No.: US 2014/0127708 A1
(43) Pub. Date: May 8, 2014

(54) PREDICTIVE BIOMARKERS FOR PROSTATE CANCER

(71) Applicant: Patrick J. Muraca, Pittsfield, MA (US)

(72) Inventor: Patrick J. Muraca, Pittsfield, MA (US)

(73) Assignee: NUCLEA BIOTECHNOLOGIES, INC., Pittsfield, MA (US)

(21) Appl. No.: 14/115,430

(22) PCT Filed: May 8, 2012
(86) PCT No.: PCT/US12/36904

§ 371 Date: Dec. 16, 2013

Related U.S. Application Data
(60) Provisional application No. 61/484,271, filed on May 10, 2011.
(60) Provisional application No. 61/551,500, filed on Oct. 26, 2011.

(51) Int. Cl. G01N 33/574

(52) U.S. Cl. CPC G01N 33/57434
USPC 435/7.1

(57) ABSTRACT

The invention relates to compositions and methods for detecting, screening, diagnosing or determining the progression of, regression of and/or survival from a proliferative disease or condition, specifically prostate cancer. The invention also provides new assays and kits for the staging or stratifying prostate cancer patients or patients suspected of having prostate cancer.

### CROSS REFERENCE TO RELATED APPLICATIONS

This application claims priority of U.S. Provisional Application No. 61/484,271 filed May 10, 2011 and U.S. Provisional Application No. 61/551,500 filed Oct. 26, 2011 each of which are incorporated by reference in their entirety.

### REFERENCE TO SEQUENCE LISTING

The present application is being filed along with a Sequence Listing in electronic format. The Sequence Listing is provided as a file entitled 2015.1004PCT_SEQLST_ST25.txt, created on May 8, 1012, which is 72,693 bytes in size. The information in the electronic format of the sequence listing is incorporated herein by reference in its entirety.

### FIELD OF THE INVENTION

The invention relates to compositions, methods, assays and kits for detecting, screening, diagnosing or determining the progression of, regression of and/or survival from a proliferative disease or condition.

### BACKGROUND OF THE INVENTION

According to the American Cancer Society, in 2009 there were over 190,000 cases of prostate cancer reported and over 27,000 related deaths in the United States. Despite advances in detection which include the use of PSA (prostate specific antigen) as a biomarker and improved clinical management, there remains a long felt need for clinical tools in the areas of prediction, patient stratification and optimization of treatment regimens.

Prostate-specific antigen (PSA) is used as a biological or tumor marker to detect prostate disease. PSA is a protein produced by cells of the prostate gland. The PSA test measures the level of PSA in the blood; but PSA alone is not a reliable indicator of the presence of prostate disease.

It is normal for men to have a low level of PSA in their blood. The reference range of PSA is between 0-4.0 ng/mL, based on a study that found 99% of a cohort of apparently healthy men had a total PSA level below 4 ng/mL. The upper limit of normal is much less than 4 ng/mL. Increased levels of PSA may suggest the presence of prostate cancer; however, prostate cancer can also be present in the complete absence of an elevated PSA level, in which case the test result would be a false negative.

Men that have elevated PSA levels typically undergo biopsy to assess the potential presence of prostate cancer. Following biopsy, histopathological grading of prostate tissue is performed by Gleason scoring, which classifies tumors from 1 to 5 (most to least differentiated) based on their most prevalent architecture, and assigns a combined score that is the sum of the two most common patterns. Patients are also diagnosed by the status of their primary tumors, from organ-confined to fully invasive (T1-4), with or without lymph node involvement (N0 or 1), and the presence and degree of distant metastases (M0 and 1a-c).

If prostate cancer is diagnosed, conventional treatment regimens include surgical excision of the prostate or irradiation methods. In the case of advanced cancer, these regimens are usually followed or substituted with androgen ablation therapy, which initially will reduce tumor burden and/or circulating PSA to low or undetectable levels, but ultimately the disease will recur in most cases.

In prostate cancer, fatty acid synthase (FAS), a 270 kDa cytosolic protein, is overexpressed throughout the natural history of a majority of tumors beginning with prostatic intraepithelial neoplasia (PIN). The protein is expressed in low to undetectable levels in most normal human tissues. Although the biochemical and metabolic basis for FAS overexpression in tumor cells in not well understood, it appears that FAS overexpression confers a selective growth advantage to tumor cells. Prostate tumors expressing high FAS levels display aggressive biologic behavior and overexpression has been associated with poor prognosis.

As men age, both benign prostate conditions and prostate cancer become more common, resulting in an increase in PSA levels. PSA levels can be increased by conditions including prostate infection, irritation or benign prostatic hyperplasia (BPH).

However, according to the National Cancer Institute, PSA levels alone do not give doctors enough information to distinguish between benign prostate conditions and cancer. Treatment needs to be individualized based on the patient's risk of progression as well as the likelihood of success and the risks of the treatment.

### SUMMARY OF THE INVENTION

The present invention provides methods for stratifying prostate cancer patients comprising the steps of determining the level of expression of the FAS gene in samples obtained from the patients; and the patients based on the level or grade of expression of the FAS gene. Stratification may be against FAS alone or in combination with other genes. One embodiment involves the combination of FAS and USP2a measurements.

The methods of the present invention may further comprise determining the level of prostate specific antigen (PSA) in a sample from said prostate cancer patient. The PSA measured may be free or total or both.

According to the present invention, stratification may be along one or more clinical management parameters which may include patient survival in years, early recurrence of the cancer, late recurrence of the cancer, disease related death, degree of cancer regression, metastasis, responsiveness to treatment, effectiveness of treatment, and /or likelihood of progression to prostate cancer.

In one embodiment of the invention is provided a method of predicting a clinical outcome of a patient diagnosed with prostate cancer, where the method comprises determining the level of expression of the FAS gene, and correlating one or more clinical management parameters with the level of expression of the FAS gene, wherein a FAS level of 3 is correlated with a negative clinical outcome. The method may be performed by determining the level of FAS alone or in combination with one or more other genes, including but not limited to, USP2a, NPY, AMACR, and pAKT. The measurements of expression level may be of RNA or protein levels of the gene, or a combination thereof

The present invention provides methods, assays and kits for practicing the invention. These include assays which involve immunohistochemical techniques and may involve the use of antibodies which may be labeled with a detectable label.

In one embodiment, the present invention provides a method for predicting the of likelihood of early or late recurrence of prostate cancer independent of tumor size, tumor grade or androgen receptor status in a patient who was or is under a course of therapeutic treatment, where the method comprises determining the level of expression of the FAS gene alone or in combination with one or more marker genes in tissue or serum in a sample obtained from said patient; and predicting the likelihood of recurrence of the cancer in the patient based on the determined level of expression of the FAS gene in tissue or serum in a sample obtained from said patient.

In one embodiment, the present invention provides a method for predicting the of likelihood of disease related death independent of tumor size, tumor grade or androgen receptor status in a patient who was or is under a course of therapeutic treatment, where the method comprises determining the level of expression of the FAS gene in tissue or serum in a sample obtained from said patient and predicting disease related death in the patient based on the determined level of expression of the FAS gene in tissue or serum in a sample obtained from said patient.

In one embodiment, a method is provided for the binary stratification of a subject suspected of having prostate cancer comprising determining the level or grade of one or more predictor variables in a sample from the subject and then stratifying the subject as likely to survive at least 5 years based on the level or grade of said one or more predictor variables, wherein the grade of the predictor variable is less than 3. The predictor variables may be selected from any of those known in the art as well as those taught herein. They may also be selected from the group consisting of FAS, NPY, USP2a, and AMACR. The subject may have been previously screened for one or more cancer markers such as PSA. The subject may also have previously had one or more biopsies of the prostate and the biopsy may have been evaluated for cancer staging by the Gleason system.

### BRIEF DESCRIPTION OF THE FIGURES

FIG. 1 is a residual quintile plot of the survival time equation showing the residuals (=observed-predicted).

FIG. 2 is a distribution profiler diagram for a patient with FAS=0, Gleason=7, PSA=13, Months of therapy=3.7, and Time (of failure)=2.

FIG. 3 is a distribution profiler diagram for a patient with FAS=1, Gleason=7, PSA=13, Months of therapy=3.7, and Time (of failure)=2.

FIG. 4 is a distribution profiler diagram for a patient with FAS=2, Gleason=7, PSA=13, Months of therapy=3.7, and Time (of failure)=2.

FIG. 5 is a distribution profiler diagram for a patient with FAS=3, Gleason=7, PSA=13, Months of therapy=3.7, and Time (of failure)=2.

FIG. 6 shows the ROC curves for the six X variables FAS, Gleason, Pre-PSA, USP2a, AMACR and NPY.

FIG. 7 is a series of plots showing failure probabilities (no survival). Each plot has Survival Time as the X-axis (from 0 to 5) and the Failure Probabilities on the Y-axis.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of methods featured in the invention, suitable methods and materials are described below.

### Definitions

For convenience, the meaning of certain terms and phrases employed in the specification, examples, and appended claims are provided below. The definitions are not meant to be limiting in nature and serve to provide a clearer understanding of certain aspects of the present invention.

The term “genome” is intended to include the entire DNA complement of an organism, including the nuclear DNA component, chromosomal or extrachromosomal DNA, as well as the cytoplasmic domain (e.g., mitochondrial DNA).

The term “gene” refers to a nucleic acid sequence that comprises control and most often coding sequences necessary for producing a polypeptide or precursor. Genes, however, may not be translated and instead code for regulatory or structural RNA molecules.

A gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions.

The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

The terms “gene expression profile” or “GEP” or “gene signature” refer to a group of genes expressed by a particular cell or tissue type wherein presence of the genes or transcriptional products thereof, taken individually (as with a single gene marker) or together or the differential expression of such, is indicative/predictive of a certain condition.

The phrase “single-gene marker” or “single gene marker” refers to a single gene (including all variants of the gene) expressed by a particular cell or tissue type wherein presence of the gene or transcriptional products thereof, taken individually the differential expression of such, is indicative/predictive of a certain condition.

The phrase “gene-protein expression profile “GPEP” as used herein refers to the group of genes and proteins expressed by a particular cell or tissue type wherein presence of the genes and the proteins, taken together or the differential expression of such, is indicative/predictive of a certain condition. GPEPs are comprised of one or more sets of GEPs and PEPs.

The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like.

The term “complementary” as it relates to nucleic acids refers to hybridization or base pairing between nucleotides or nucleic acids, such as, for example, between the two strands of a double-stranded DNA molecule or between an oligonucleotide probe and a target are complementary.

As used herein, an “expression product” is a biomolecule, such as a protein or mRNA, which is produced when a gene in an organism is expressed. An expression product may comprise post-translational modifications. The polypeptide of a gene may be encoded by a full length coding sequence or by any portion of the coding sequence.

The terms “amino acid” and “amino acids” refer to all naturally occurring L-alpha-amino acids. The amino acids are identified by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

The term “amino acid sequence variant” refers to molecules with some differences in their amino acid sequences as compared to a native sequence. The amino acid sequence variants may possess substitutions, deletions, and/or insertions at certain positions within the amino acid sequence. Ordinarily, variants will possess at least about 70% homology to a native sequence, and preferably, they will be at least about 80%, more preferably at least about 90% homologous to a native sequence.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions or deletions of amino acid residues that still maintain the properties of the parent polypeptide.

The term “derivative” is used synonymously with the term “variant” and refers to a molecule that has been modified or changed in any way relative to a reference molecule or starting molecule.

The present invention contemplates several types of compositions, such as antibodies, which are amino acid based including variants and derivatives. These include substitutional, insertional, deletion and covalent variants and derivatives. As such, included within the scope of this invention are polypeptide based molecules containing substitutions, insertions and/or additions, deletions and covalently modifications. For example, sequence tags or amino acids, such as one or more lysines, can be added to the polypeptide sequences of the invention (e.g., at the N-terminal or C-terminal ends). Sequence tags can be used for polypeptide purification or localization. Lysines can be used to increase solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence “Immediately adjacent” to an amino acid means connected to either the alpha-carboxy or alpha-amino functional group of the amino acid.

“Deletional variants,” when referring to proteins, are those with one or more amino acids in the native or starting amino acid sequence removed. Ordinarily, deletional variants will have one or more amino acids deleted in a particular region of the molecule.

“Covalent derivatives,” when referring to proteins, include modifications of a native or starting protein with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present invention.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W. H. Freeman & Co., San Francisco, pp. 79-86 (1983)).

Covalent derivatives specifically include fusion molecules in which proteins of the invention are covalently bonded to a non-proteinaceous polymer. The non-proteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol. The proteins may be linked to various non-proteinaceous polymers, such as polyethylene glycol, polypropylene glycol or polyoxyalkylenes, in the manner set forth in U.S. Pat. No. 4,640,835; 4,496,689; 4,301,144; 4,670,417; 4,791,192 or 4,179,337.

“Features” when referring to proteins are defined as distinct amino acid sequence-based components of a molecule. Features of the proteins of the present invention include surface manifestations, local conformational shape, folds, loops, half-loops, domains, half-domains, sites, termini or any combination thereof.

As used herein when referring to proteins the term “surface manifestation” refers to a polypeptide based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830; 1997).

As used herein when referring to proteins the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions).

As used herein when referring to proteins the term “half-domain” means portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid based embodiments is used synonymous with “amino acid residue” and “amino acid side chain”. A site represents a position within a peptide or polypeptide that may be modified, manipulated, altered, derivatized or varied within the polypeptide based molecules of the present invention.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

A “protein” means a polymer of amino acid residues linked together by peptide bonds. The term, as used herein, refers to proteins, polypeptides, and peptides of any size, structure, or function. Typically, however, a protein will be at least 50 amino acids long. In some instances the protein encoded is smaller than about 50 amino acids. In this case, the polypeptide is termed a peptide. If the protein is a short peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex. The term protein may also apply to amino acid polymers in which one or more amino acid residues is an artificial chemical analogue of a corresponding naturally occurring amino acid.

The term “protein expression” refers to the process by which a nucleic acid sequence undergoes translation such that detectable levels of the amino acid sequence or protein are expressed.

The terms “protein expression profile” or “PEP” or “protein expression signature” refer to a group of proteins expressed by a particular cell or tissue type (e.g., neuron, coronary artery endothelium, or diseased tissue), wherein presence of the proteins taken individually (as with a single protein marker) or together or the differential expression of such proteins, is indicative/predictive of a certain condition.

The phrase “single-protein marker” or “single protein marker” refers to a single protein (including all variants of the protein) expressed by a particular cell or tissue type wherein presence of the protein or translational products of the gene encoding said protein, taken individually the differential expression of such, is indicative/predictive of a certain condition.

A “fragment of a protein,” as used herein, refers to a protein that is a portion of another protein. For example, fragments of proteins may comprise polypeptides obtained by digesting full-length protein isolated from cultured cells. In one embodiment, a protein fragment comprises at least about six amino acids. In another embodiment, the fragment comprises at least about ten amino acids. In yet another embodiment, the protein fragment comprises at least about sixteen amino acids.

The terms “array” and “microarray” refer to any type of regular arrangement of objects usually in rows and columns. As it relates to the study of gene and/or protein expression, arrays refer to an arrangement of probes (often oligonucleotide or protein based) or capture agents anchored to a surface which are used to capture or bind to a target of interest. Targets of interest may be genes, products of gene expression, and the like. The type of probe (nucleic acid or protein) represented on the array is dependent on the intended purpose of the array (e.g., to monitor expression of human genes or proteins). The oligonucleotide- or protein-capture agents on a given array may all belong to the same type, category, or group of genes or proteins. Genes or proteins may be considered to be of the same type if they share some common characteristics such as species of origin (e.g., human, mouse, rat); disease state (e.g., cancer); structure or functions (e.g., protein kinases, tumor suppressors); or same biological process (e.g., apoptosis, signal transduction, cell cycle regulation, proliferation, differentiation). For example, one array type may be a “cancer array” in which each of the array oligonucleotide- or protein-capture agents correspond to a gene or protein associated with a cancer. An “epithelial array” may be an array of oligonucleotide- or protein-capture agents corresponding to unique epithelial genes or proteins. Similarly, a “cell cycle array” may be an array type in which the oligonucleotide- or protein-capture agents correspond to unique genes or proteins associated with the cell cycle.

The terms “immunohistochemical” or as abbreviated “IHC” as used herein refer to the process of detecting antigens (e.g., proteins) in a biologic sample by exploiting the binding properties of antibodies to antigens in said biologic sample.

The term “immunoassay” refers to a test that uses the binding of antibodies to antigens to identify and measure certain substances. Immunoassays often are used to diagnose disease, and test results can provide information about a disease that may help in planning treatment (for example, when estrogen receptors are measured in prostate cancer). An immunoassay takes advantage of the specific binding of an antibody to its antigen. Monoclonal antibodies are often used as they usually bind only to one site of a particular molecule, and therefore provide a more specific and accurate test, which is less easily confused by the presence of other molecules. The antibodies used must have a high affinity for the antigen of interest, because a very high proportion of the antigen must bind to the antibody in order to ensure that the assay has adequate sensitivity.

The term “PCR” or “RT-PCR”, abbreviations for polymerase chain reaction technologies, as used here refer to techniques for the detection or determination of nucleic acid levels, whether synthetic or expressed.

The term “cell type” refers to a cell from a given source (e.g., a tissue, organ) or a cell in a given state of differentiation, or a cell associated with a given pathology or genetic makeup.

The term “activation” as used herein refers to any alteration of a signaling pathway or biological response including, for example, increases above basal levels, restoration to basal levels from an inhibited state, and stimulation of the pathway above basal levels.

The term “differential expression” refers to both quantitative as well as qualitative differences in the temporal and tissue expression patterns of a gene or a protein in diseased tissues or cells versus normal adjacent tissue. For example, a differentially expressed gene may have its expression activated or completely inactivated in normal versus disease conditions, or may be up-regulated (over-expressed) or down-regulated (under-expressed) in a disease condition versus a normal condition. Such a qualitatively regulated gene may exhibit an expression pattern within a given tissue or cell type that is detectable in either control or disease conditions, but is not detectable in both. Stated another way, a gene or protein is differentially expressed when expression of the gene or protein occurs at a higher or lower level in the diseased tissues or cells of a patient relative to the level of its expression in the normal (disease-free) tissues or cells of the patient and/or control tissues or cells.

The term “detectable” refers to an RNA expression pattern which is detectable via the standard techniques of polymerase chain reaction (PCR), reverse transcriptase-(RT) PCR, differential display, and Northern analyses, or any method which is well known to those of skill in the art. Similarly, protein expression patterns may be “detected” via standard techniques such as Western blots.

The term “complementary” as it relates to arrays refers to the topological compatibility or matching together of the interacting surfaces of a probe molecule and its target. The target and its probe can be described as complementary, and furthermore, the contact surface characteristics are complementary to each other.

The term “antibody” means an immunoglobulin, whether natural or partially or wholly synthetically produced. All derivatives thereof that maintain specific binding ability are also included in the term. The term also covers any protein having a binding domain that is homologous or largely homologous to an immunoglobulin binding domain. An antibody may be monoclonal or polyclonal. The antibody may be a member of any immunoglobulin class, including any of the human classes: IgG, IgM, IgA, IgD, and IgE, etc.

The term “antibody fragment” refers to any derivative or portion of an antibody that is less than full-length. In one aspect, the antibody fragment retains at least a significant portion of the full-length antibody's specific binding ability, specifically, as a binding partner. Examples of antibody fragments include, but are not limited to, Fab, Fab′, F(ab′)2, scFv, Fv, dsFv diabody, and Fd fragments. The antibody fragment may be produced by any means. For example, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may comprise a single chain antibody fragment. In another embodiment, the fragment may comprise multiple chains that are linked together, for example, by disulfide linkages. The fragment may also comprise a multimolecular complex. A functional antibody fragment may typically comprise at least about 50 amino acids and more typically will comprise at least about 200 amino acids.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. This type of antibodies is produced by the daughter cells of a single antibody-producing hybridoma. A monoclonal antibody typically displays a single binding affinity for any epitope with which it immunoreacts.

The modifier “monoclonal” indicates the character of the antibody as being obtained from a substantially homogeneous population of antibodies, and is not to be construed as requiring production of the antibody by any particular method. Monoclonal antibodies recognize only one type of antigen The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies. The preparation of antibodies, whether monoclonal or polyclonal, is know in the art. Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999.

A monoclonal antibody may contain an antibody molecule having a plurality of antibody combining sites, each immunospecific for a different epitope, e.g., a bispecific monoclonal antibody. Monoclonal antibodies may be obtained by methods known to those skilled in the art. Kohler and Milstein (1975), Nature, 256:495-497; U.S. Pat. No. 4,376,110; Ausubel et al. (1987, 1992), eds., Current Protocols in Molecular Biology, Greene Publishing Assoc. and Wiley Interscience, N.Y.; Harlow and Lane (1988), Antibodies: A Laboratory Manual, Cold Spring Harbor Laboratory; Colligan et al. (1992, 1993), eds., Current Protocols in Immunology, Greene Publishing Assoc. and Wiley Interscience, N.Y.; Iyer et al., Ind. J. Med. Res., (2000), 123:561-564.

An “antibody preparation” is meant to embrace any composition in which an antibody may be present, e.g., a serum (antiserum).

Antibodies may be labeled with detectable labels by one of skill in the art. The label can be a radioisotope, fluorescent compound, chemiluminescent compound, enzyme, or enzyme co-factor, or any other labels known in the art. In some aspects, the antibody that binds to an entity one wishes to measure (the primary antibody) is not labeled, but is instead detected by binding of a labeled secondary antibody that specifically binds to the primary antibody.

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), intracellularly made antibodies (i.e., intrabodies), and epitope-binding fragments of any of the above. The antibodies of the invention can be from any animal origin including birds and mammals. Preferably, the antibodies are of human, murine (e.g., mouse and rat), donkey, sheep, rabbit, goat, guinea pig, camel, horse, or chicken origin.

Multispecific antibodies can be specific for different epitopes of a peptide of the present invention, or can be specific for both a peptide of the present invention, and a heterologous epitope, such as a heterologous peptide or solid support material. See, e.g., WO 93/17715; WO 92/08802; WO 91/00360; WO 92/05793; Tutt et al., 1991, J. Immunol., 147:60-69; U.S. Pat. Nos. 4,474,893; 4,714,681; 4,925,648; 5,573,920; 5,601,819; and Kostelny et al., 1992, J. Immunol., 148:1547-1553. For example, the antibodies may be produced against a peptide containing repeated units of a FAS peptide sequence of the invention, or they may be produced against a peptide containing two or more FAS peptide sequences of the invention, or the combination thereof.

Moreover, antibodies can also be prepared from any region of the FAS peptides of the invention. In addition, if a polypeptide is a receptor protein, antibodies can be developed against an entire receptor or portions of the receptor, for example, an intracellular domain, an extracellular domain, the entire transmembrane domain, specific transmembrane segments, any of the intracellular or extracellular loops, or any portions of these regions. Antibodies can also be developed against specific functional sites, such as the site of ligand binding, or sites that are glycosylated, phosphorylated, myristylated, or amidated, for example.

By “amplification” is meant production of multiple copies of a target nucleic acid that contains at least a portion of an intended specific target nucleic acid sequence (FAS, USP2a, AMACR, etc). The multiple copies may be referred to as amplicons or amplification products. Preferably, the amplified target contains less than the complete target gene sequence (introns and exons) or an expressed target gene sequence (spliced transcript of exons and flanking untranslated sequences). For example, FAS-specific amplicons may be produced by amplifying a portion of the FAS target polynucleotide by using amplification primers which hybridize to, and initiate polymerization from, internal positions of the FAS target polynucleotide. Preferably, the amplified portion contains a detectable target sequence which may be detected using any of a variety of well known methods.

By “primer” or “amplification primer” is meant an oligonucleotide capable of binding to a region of a target nucleic acid or its complement and promoting nucleic acid amplification of the target nucleic acid. In most cases a primer will have a free 3′ end which can be extended by a nucleic acid polymerase. All amplification primers include a base sequence capable of hybridizing via complementary base interactions either directly with at least one strand of the target nucleic acid or with a strand that is complementary to the target sequence. Amplification primers serve as substrates for enzymatic activity that produces a longer nucleic acid product.

A “target-binding sequence” of an amplification primer is the portion that determines target specificity because that portion is capable of annealing to a target nucleic acid strand or its complementary strand. The complementary target sequence to which the target-binding sequence hybridizes is referred to as a primer-binding sequence.

By “detecting” an amplification product is meant any of a variety of methods for determining the presence of an amplified nucleic acid, such as, for example, hybridizing a labeled probe to a portion of the amplified product. A labeled probe is an oligonucleotide that specifically binds to another sequence and contains a detectable group which may be, for example, a fluorescent moiety, a chemiluminescent moiety, a radioisotope, biotin, avidin, enzyme, enzyme substrate, or other reactive group.

By “nucleic acid amplification conditions” is meant environmental conditions including salt concentration, temperature, the presence or absence of temperature cycling, the presence of a nucleic acid polymerase, nucleoside triphosphates, and cofactors which are sufficient to permit the production of multiple copies of a target nucleic acid or its complementary strand using a nucleic acid amplification method. Many well-known methods of nucleic acid amplification require thermocycling to alternately denature double-stranded nucleic acids and hybridize primers.

The term “biomarker” as used herein refers to a substance indicative of a biological state. According to the present invention, biomarkers include the GPEPs, PEPs, GEPs or combinations thereof. Biomarkers according to the present invention also include any compounds or compositions which are used to identify or signal the presence of one or more members of the GPEPs, PEPs, GEPs or combinations thereof disclosed herein. For example, an antibody created to bind to any of the proteins identified as a member of a PEP herein, may be considered useful as a biomarker, although the antibody itself is a secondary indicator.

The term “biological sample” or “biologic sample” refers to a sample obtained from an organism (e.g., a human patient) or from components (e.g., cells) or from body fluids (e.g., blood, serum, sputum, urine, etc) of an organism. The sample may be of any biological tissue, organ, organ system or fluid. The sample may be a “clinical sample” which is a sample derived from a patient. Such samples include, but are not limited to, sputum, blood, blood cells (e.g., white cells), amniotic fluid, plasma, semen, bone marrow, and tissue or core, fine or punch needle biopsy samples, urine, peritoneal fluid, and pleural fluid, or cells therefrom. Biological samples may also include sections of tissues such as frozen sections taken for histological purposes. A biological sample may also be referred to as a “patient sample.”

The term “condition” refers to the status of any cell, organ, organ system or organism. Conditions may reflect a disease state or simply the physiologic presentation or situation of an entity. Conditions may be characterized as phenotypic conditions such as the macroscopic presentation of a disease or genotypic conditions such as the underlying gene or protein expression profiles associated with the condition. Conditions may be benign or malignant.

The term “cancer” in an individual refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an individual, or may circulate in the blood stream as independent cells, such as leukemic cells.

The term “prostate cancer” means a cancer of the prostate tissue.

The term “cell growth” is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

The term “tumor growth” or “tumor metastases growth”, as used herein, unless otherwise indicated, is used as commonly used in oncology, where the term is principally associated with an increased mass or volume of the tumor or tumor metastases, primarily as a result of tumor cell growth.

The term “metastasis” means the process by which cancer spreads from the place at which it first arose as a primary tumor to distant locations in the body. Metastasis also refers to cancers resulting from the spread of the primary tumor. For example, someone with prostate cancer may show metastases in their lymph system, liver, bones or lungs.

The term “lesion” or “lesion site” as used herein refers to any abnormal, generally localized, structural change in a bodily part or tissue. Calcifications or fibrocystic features are examples of lesions of the present invention.

The term “clinical management parameter” refers to a metric or variable considered important in the detecting, screening, diagnosing, staging or stratifying patients, or determining the progression of, regression of and/or survival from a disease or condition. Examples of such clinical management parameters include, but are not limited to survival in years, disease related death, early or late recurrence, degree of regression, metastasis, responsiveness to treatment, effectiveness of treatment or the likelihood of progression to prostate cancer.

The term “endpoint” means a final stage or occurrence along a path or progression.

The term “tumor assessment endpoint” means an endpoint observation or calculation based on the stage, status or occurrence of a tumor. Examples of endpoints based on tumor assessments include, but are not limited to, survival, disease free survival (DFS), objective response rate (ORR), time to progression (TTP), progression free survival (PFS), and time to treatment failure (TTF).

The term “treating” as used herein, unless otherwise indicated, means reversing, alleviating, inhibiting the progress of, or preventing, either partially or completely, the growth of tumors, tumor metastases, or other cancer-causing or neoplastic cells in a patient with cancer. The term “treatment” as used herein, unless otherwise indicated, refers to the act of treating.

The phrase “a method of treating” or its equivalent, when applied to, for example, cancer refers to a procedure or course of action that is designed to reduce, eliminate or prevent the number of cancer cells in an individual, or to alleviate the symptoms of a cancer. “A method of treating” cancer or another proliferative disorder does not necessarily mean that the cancer cells or other disorder will, in fact, be completely eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an individual, is nevertheless deemed an overall beneficial course of action.

The term “predicting” means a statement or claim that a particular event will, or is very likely to, occur in the future.

The term “prognosing” means a statement or claim that a particular biologic event will, or is very likely to, occur in the future.

The term “progression” or “cancer progression” means the advancement or worsening of or toward a disease or condition.

The term “regression” or “degree of regression” refers to the reversal, either phenotypically or genotypically, of a cancer progression. Slowing or stopping cancer progression may be considered regression.

The term “stratifying” as it relates to patients means the parsing of patients into groups of predicted outcomes along a continuum of from a positive outcome (such as disease free) to moderate or good outcomes (such as improved quality of life or increased survival) to poor outcomes (such as terminal prognosis or death).

The term “therapeutically effective agent” means a composition that will elicit the biological or medical response of a tissue, organ, system, organism, animal or human that is being sought by the researcher, veterinarian, medical doctor or other clinician.

The term “therapeutically effective amount” or “effective amount” means the amount of the subject compound or combination that will elicit the biological or medical response of a tissue, organ, system, organism, animal or human that is being sought by the researcher, veterinarian, medical doctor or other clinician.

The term “correlate” or “correlation” as used herein refers to a relationship between two or more random variables or observed data values. A correlation may be statistical if, upon analysis by statistical means or tests, the relationship is found to satisfy the threshold of significance of the statistical test used.

### Clinical Management Parameters

The invention relates to compositions, methods and assays for detecting, screening for, or diagnosing prostate cancer; staging or stratifying prostate cancer patients; and determining the progression of, regression of and/or survival from prostate cancer.

In doing so, the present invention provides methods, algorithms and other clinical tools to augment traditional diagnostic, prognostic and/or therapeutic paradigms. Combination approaches using one or more biomarkers in the determination of the value of one or more clinical management parameters also are envisioned. For example methods of this invention that measure both FAS and PSA biomarkers can provide potentially superior results to diagnostic assays measuring just one of these biomarkers, as illustrated by the data presented herein. This dual or multi-biomarker approach, in combination with imaging techniques would provide even further superiority. Any dual, or multiple, biomarker approach (with or without companion imaging) thus reduces the number of patients that are predicted not to benefit from treatment, and thus potentially reduces the number of patients that fail to receive treatment that may extend their life significantly.

Clinical management parameters addressed by the present invention include survival in years, disease related death, early or late recurrence, degree of regression, metastasis, responsiveness to treatment, effectiveness of treatment and Gleason score. Also included are measurements of PSA for comparison.

It is very important to distinguish patients who will develop cancer to those who will not. There is a lack of data regarding the natural history of untreated low-risk, low-PSA prostate cancer in healthy men in their 60s and 70s. Studies at autopsy have shown that a third of men ages 40-60 have lived with prostate cancer. This number grows to 75% after age 85. Yet, only 3% of men die from the disease and there remains no definitive test.

Advantageously, practice of the present invention can result in reduced harms caused by screening (resulting in false positives) and the unnecessary subsequent evaluations and therapy (e.g., radiation, biopsy, hormones and surgery), including infections or urinary retention or incontinence, unnecessary screening-triggered biopsies, erectile dysfunction, rectal and/or urethral injury, persistent blood in the semen, breast enlargement, hot flashes, impotence and/or an overall reduced quality of life. The present invention provides methods of reducing, avoiding or eliminating harms resulting from false-positive treatment regimens in patients that would have undergone radical therapy such as radiation or surgery. To this end, the invention provides a mechanism by which men who have been screened and found to have elevated PSA levels, may be screened or tested for one or more of the predictor variables described herein before undergoing radiation, biopsy or surgery. This confirmation assay or “Survive5” test, as demonstrated herein, provides a better predictor of survival than current PSA measurements or Gleason scores.

Having found that FAS expression is a superior predictor of many of the clinical management parameters important to clinicians treating patients having or suspected of having prostate cancer, the present invention involves the rapid and accurate identification of FAS expression in tissue, cells and/or serum.

The method generally comprises the following steps: (a) obtaining a biological sample (optimally containing cells or other cell or fluid) from a cancer patient; (b) contacting the sample with a detection agent specific for FAS; (c) detecting the presence, amount or levels of FAS in (b); and (d) correlating the presence, amount or levels of FAS (alone or in combination) with the one or more clinical management parameters in order to aid in the prevention, diagnosis or treatment of prostate cancer.

The biological sample may be cells or tissue, and preferably is serum or plasma containing cells. However, the cells also may be obtained from tissue samples or cell cultures such as in ex vivo or in situ methods.

The detection agent may a nucleic acid probe specific for FAS, or an anti-FAS antibody.

### FAS Probes

The present invention provides novel nucleic acid based probes useful in the detection of the FAS gene or protein in a biological sample. To this end, the present invention includes nucleic acid sequences specific for segments of a human FAS gene which are used in methods of detecting FAS-specific sequences in nucleic acids prepared from a biological sample. The invention further includes nucleic acid sequences specific for segments of other prostate-associated genetic markers, a human PSA, USP2a, pAKT, NPY, and/or AMACR, which are used in methods of detecting prostate-associated sequences that are useful cancer detection markers in nucleic acids prepared from a biological sample of tissue or fluid from a patient with prostate cancer. The sample may be prostate tissue or non-prostate tissue. The non-prostate tissue can include, for example, blood, lymph node, breast or breast cyst, kidney, liver, lung, muscle, stomach or intestinal tissue. The invention also includes preferred methods that combine nucleic acid sequences for amplifying and detecting FAS-specific sequences, PSA, USP2a, pAKT, NPY, and/or AMACR sequences, individually or in combination.

Preferred probes, primers and promoter-primers of the present invention used for detecting

The present invention also includes a method for detecting and quantifying the FAS-specific RNA species. Other embodiments of the invention include methods for detecting PSA, USP2a, pAKT, NPY, and/or AMACR RNA species, individually or in combination with each other or FAS sequences. Moreover, detection of these markers individually and in combination, are clinically important because cancers from individual patients may express one or more of the markers, such that detecting one or more of the markers decreases the potential of false negatives during diagnosis that might otherwise result if the presence of only one marker was tested.

In one embodiment, commercial antibodies may be used to detect expression. One such antibody for USP2a is the USP2 Antibody (N-term) from Abgent (San Diego, Calif.; Cat. #AP2131a).

### In Situ Hybridization (ISH) and Fluorescence In Situ Hybridization (FISH)

The present invention provides methods of detecting target nucleic acids via in situ hybridization and fluorescent in situ hybridization using novel probes. The methods of in situ hybridization were first developed in 1969 and many improvements have been made since. The basic technique utilizes hybridization kinetics for RNA and/or DNA via hydrogen bonding. By labeling sequences of DNA or RNA of sufficient length (approximately 50-300 base pairs), selective probes can be made to detect particular sequences of DNA or RNA. The application of these probes to tissue sections allows DNA or RNA to be localized within tissue regions and cell types. Methods of probe design are known to those of skill in the art. Detection of hybridized probe and target may be performed in several ways known in the art. Most prominently is through the use of detection labels attached to the probes. Probes of the present invention may be single or double stranded and may be DNA, RNA, or mixtures of DNA and RNA. They may also constitute any nucleic acid based construct. Labels for the probes of the present invention may be radioactive or non-radioactive and the design and use of such labels is well known in the art.

### FAS Antibodies

In one embodiment, the present invention utilizes anti-FAS antibodies and ELISA assay. The anti-FAS antibodies preferably are those disclosed in PCT Publication PCT/US2010/030545 published Oct. 14, 2010, and PCT/US2010/046773 published Mar. 17, 2011, respectively.

The antibodies used in the present invention for detection or capture of FAS are novel anti-FAS antibodies that are highly specific for human FAS.

In one embodiment, commercial antibodies for the detection of FAS are used. For IHC the antibodies which may be used are the human anti-FASN Antibody, Affinity Purified (Catalog No. A301-324A) from Bethyl Laboratories (Montgomery, Tex.) and for ELISA studies, antibodies which may be used include the Fatty Acid Synthase Antibody Pair (Catalog No. H00002194-AP11) from Novus Biologicals (Littleton, Colo.). The pair contains a Capture antibody which is rabbit affinity purified polyclonal anti-FASN (100 ug) and a Detection antibody which is mouse monoclonal anti-FASN, IgG1 Kappa (20 ug).

In one embodiment, the present antibodies are monoclonal antibodies specific for a human FAS sequence selected from SEQ ID NOs. 1-5 (Table 1). In another embodiment, the present antibodies are used as capture antibodies in a sandwich ELISA assay.

TABLE 1
FAS Peptides

| Hybridoma | FAS Peptide | SEQ ID |
|---|---|---|
| A | VAQGQWEPSGXAP | 1 |
| B | PSGPAPTNXGALE | 2 |
| C | TLEQQHXVAQGQW | 3 |
| D | EVDPGSAELQKVLQGD | 4 |
| E | ELSSKADEASELAC | 5 |

### FAS Antibodies and Detection Rate

In one embodiment, the FAS antibodies disclosed herein may be used in the detection of prostate cancer, either alone or in combination with measurements of PSA. Measurements may be made in tissue, cells or serum of patients.

### Gleason Score and FAS

In the practice of the methods of the invention, FAS expression may be combined with one or more clinical management parameters to provide improvements in the diagnosis, care and/or treatment of the patients. One such combination contemplated by the present invention is with Gleason score. Gleason scores or grades are defined by a primary or predominant tissue pattern and a secondary pattern of tissue presentation (See Table 2). Each of the two patterns is given a score and the scores are combined for a final Gleason score.

TABLE 2
Gleason score outline

| Pattern | |
|---|---|
| (score) | Description |
| Pattern 1 | The cancerous prostate closely resembles normal prostate |
| (score 1) | tissue. The glands are small, well-formed, and closely |
| | packed. |
| Pattern 2 | The tissue still has well-formed glands, but they are larger |
| (score 2) | and have more tissue between them. |
| Pattern 3 | The tissue still has recognizable glands, but the cells are |
| (score 3) | darker. At high magnification, some of these cells have left |
| | the glands and are beginning to invade the surrounding |
| | tissue. |
| Pattern 4 | The tissue has few recognizable glands. Many cells are |
| (score 4) | invading the surrounding tissue. |
| Pattern 5 | The tissue does not have recognizable glands. There are |
| (score 5) | often just sheets of cells throughout the surrounding tissue. |

In one embodiment, FAS levels in combination with a Gleason score of 5-7, may be used to stratify or stage patients having prostate cancer and provide prognostic information regarding survival or responsiveness to treatment. Across total Gleason scores 5-7 (inclusive), FAS grades (or levels) 0-3 have been found to collectively embrace over 88% of patient samples. See Example 12. Consequently, methods of the present invention employing both FAS grade, as a measurement for expression level, and Gleason scores of 5-7 will allow stratification of a significant number of individuals and thereby provide a more reliable prediction of survival (or failure). Along the continuum of predictive scales, a FAS level of 3 and Gleason score of 5-7 would be most predictive. It is known that a Gleason score of 7 is predictive of lethality in some patients, yet in others it is not. Therefore a combination of FAS with Gleason score provides an improved prediction method.

### Gene Expression and Localization of Expression

In one embodiment of the invention, FAS expression is measured relative to the expression of one or more additional genes and/or at one or more different biopsy sites. Comparisons of gene expression within the cancer site as compared to expression at the margin of the cancer and at sites distal from the cancer allow conclusions to be drawn about the status of a sample and whether it will become cancerous. These conclusions then allow for improved predictions about metastasis and consequently survival. One set of genes which are particularly useful in these methods includes FAS combined with one or more of USP2a, pAKT and NPY. Additional patient parameters may also be combined with the gene expression data to improve the predictive power of the method. One such patient parameter is age. For patients between the ages of 50-75, the gene expression profiles described here are more significant.

### FAS and Degree of Regression

In one embodiment, FAS expression levels are used as a predictor of probability of cancer regression which allows stratification between poor and excellent outcomes for individual patients. In this method, FAS expression is correlated with degree of regression where higher FAS expression levels are predictive of clinical outcomes. It has been determined that FAS level is an excellent predictor of poor outcomes.

### FAS and Clinical Survival

The present invention includes new methods of predicting the likelihood of survival of patients having or suspected of having prostate cancer. The predictive power of the tools provided herein have been fit to a FAS survival model (FSM) which can be used alone or in combination with other clinical factors in the management of prostate cancer.

FAS and USP2a-Differential Expression

In one embodiment, the present invention provides for the use of combinations of predictors which, heretofore, have not been known as significant collective indicator combinations. These combinations may form the basis of methods, assays or kits useful in the clinical management of prostate cancer.

### Gene/Protein Expression Profiles

Also described herein are compositions and methods for employing gene and protein expression profiles in prognosis, prediction and management of treatment paradigms associated with prostate cancer.

Positive treatment outcomes for prostate cancer depend highly on early detection and intervention. Most early detections are achieved with the use of physical examinations or assays involving measurement of prostate specific antigen (PSA). However, these techniques do not provide complete predictive power. False positives and, worse yet, false negatives may occur as a result of obscured or complicated tissue physiology and the variability of the PSA test across individuals. Consequently, these approaches have not led to improvements in long-term outcome measures such as survival. The GEPs and PEPs (collectively the GPEPs) of the present invention provides the clinician with a prognostic tool capable of providing valuable information that can positively affect management of the disease. According to the present invention, oncologists can assay the suspect tissue for the presence of members of a GPEP, and can identify with a high degree of accuracy those patients whose condition is likely to progress, regress or become a more aggressive from of the disease. This information, taken together with other available clinical information including imaging data, allows more effective management of the disease.

In one aspect of the invention, the expression of genes or proteins in a prostate tissue sample or serum from a patient is assayed using array or immunohistochemistry techniques to identify the expression of genes proteins in a GPEP.

Certain methods of the present invention comprise (a) obtaining a biological sample (preferably prostate tissue or serum) (b) contacting the sample with nucleic acid probes or antibodies specific for one or more members of a GPEP, PEP or GEP and (c) determining whether one or more of the members of the profile are up-regulated (over-expressed).

The predictive value of the GPEPs for determining the likelihood of cancer progression increases with the number of the members found to be up-regulated. Preferably, at least about two, more preferably at least about four, and most preferably about seven, of the genes and/or proteins in a GPEP are overexpressed. In a preferred embodiment, samples of normal (undiseased), margin tissue (tissue from the patient's prostate tumor capsule surrounding the lesion site) as well as other control tissues or fluids (including serum) are assayed simultaneously, using the same reagents and under the same conditions, with the primary lesion site. Preferably, expression of at least one reference proteins also is measured at the same time and under the same conditions.

In one embodiment, the present invention comprises gene expression profiles and protein expression profiles that are indicative of the likelihood of recurrence/metastasis of disease in a prostate cancer patient. In this embodiment, the present method comprises (a) obtaining a biological sample (preferably primary resected tumor or serum) of a patient afflicted with prostate cancer; (b) contacting the sample with nucleic acid probes (or antibodies) to the proteins of a PEPs and (c) determining whether two or more of the members of the profile are up-regulated (over-expressed). The predictive value of the gene profile for determining the likelihood of recurrence increases with the number of these genes that are found to be up-regulated in accordance with the invention. Preferably, at least about two, more preferably at least about four, and most preferably about seven, of the genes in a GPEP are differentially expressed. The biological sample preferably is a sample of the patient's tissue, e.g., primary resected tumor; normal (undiseased) tissue or serum from the same patient is used as a control. Preferably, expression of at least one reference gene also is measured. The currently preferred reference genes are beta-actin (ACTB), glyceraldehyde-3-phosphate dehydrogenase (GAPDH), beta glucoronidase (GUSB) as positive controls while negative controls include large ribosomal protein (RPLPO) and/or transferrin receptor (TRFC). Beta actin may be used as the positive control for IHC.

The present invention further comprises assays for determining the gene and/or protein expression profile in a patient's sample, and instructions for using the assay. The assay may be based on detection of nucleic acids (e.g., using nucleic acid probes specific for the nucleic acids of interest) or proteins or peptides (e.g., using nucleic acid probes or antibodies specific for the proteins/peptides of interest). In one embodiment, the assay comprises an immunohistochemistry (IHC) test in which tissue samples, preferably arrayed in a tissue microarray (TMA), are contacted with antibodies specific for the proteins/peptides identified in the GPEP where detection is taken as being indicative of a relationship between the detected gene and one or more clinical management parameters such as survival in years, disease related death, early or late recurrence, degree of regression, metastasis or the likelihood of progression to prostate cancer. In one embodiment, the assay comprises an immunohistochemistry (IHC) test in which serum samples, preferably arrayed in a tissue microarray (TMA), are contacted with antibodies specific for the proteins/peptides identified in the GPEP where detection is taken as being indicative of a relationship between the detected gene and one or more clinical management parameters such as survival in years, disease related death, early or late recurrence, degree of regression, metastasis or the likelihood of progression to prostate cancer.

Inclusion of any of the biomarker or diagnostic methods described herein as part of treatment and/or monitoring regimens to predict the progression to, or effectiveness of treatment of, a cancer patient with any therapeutic provides an advantage over treatment or monitoring regimens that do not include such a biomarker or diagnostic step, in that only that patient population which needs or derives most benefit from such therapy or monitoring need be treated or monitored, and in particular, patients who are predicted not to need or benefit from treatment (where progression is not predicted) with any therapy need not be treated.

The present invention further provides a method for treating a patient who may have prostate cancer, comprising the step of diagnosing a patient's likely progression to cancer using one or more GPEP signatures to predict progression; and a step of administering the patient an appropriate treatment regimen for prostate cancer given the patient's age, or other therapeutically relevant criteria.

### Determination of Gene Expression Profiles

Methods used to identify gene expression profiles indicative of whether a patient's condition is likely to progress to prostate cancer are generally described here and further described in the Examples herein. Other methods for identifying gene and/or protein expression profiles are known; any of these alternative methods also could be used See, e.g., Chen et al., NEJM, 356(1):11-20 (2007); Lu et al., PLOS Med., 3(12):e467 (2006); Wang et al., J. Clin. Oncol., 2299):1564 (2004); Golub et al., Science, 286:531-537 (1999).

In one method, parallel testing in which, in one track, those genes are identified which are over-/under-expressed as compared to normal (non-cancerous) tissue and/or disease tissue from patients that experienced different outcomes; and, in a second track, those genes are identified comprising chromosomal insertions or deletions as compared to the same normal and disease samples. These two tracks of analysis produce two sets of data. The data are analyzed and correlated using an algorithm which identifies the genes of the gene expression profile (i.e., those genes that are differentially expressed in the cancer tissue of interest). Positive and negative controls may be employed to normalize the results, including eliminating those genes and proteins that also are differentially expressed in normal tissues from the same patients, and is disease tissue having a different outcome, and confirming that the gene expression profile is unique to the cancer of interest.

As an initial step, biological samples are acquired from patients presenting with either calcifications or fibrocystic disease. Tissue samples are also obtained from patients diagnosed as having progressed to prostate cancer, including samples of the primary resected tumor, metastatic lymph nodes and normal (undiseased) marginal prostate tissue from each patient. Clinical information associated with each sample, including treatment with chemotherapeutic drugs, surgery, radiation or other treatment, outcome of the treatments and recurrence or metastasis of the disease, is recorded in a database. Clinical information also includes information such as age, sex, medical history, treatment history, symptoms, family history, recurrence (yes/no), etc. Samples of normal (non-cancerous) tissue of different types (e.g., lung, brain, prostate) as well as samples of non-prostate cancers (e.g., melanoma, breast cancer, ovarian cancer) can be used as positive controls. Samples of normal undiseased prostate tissue from a set of healthy individuals can be used as positive controls, and prostate tumor samples from patients whose cancer did recur/metastasize may be used as negative controls.

Gene expression profiles (GEPs) are then generated from the biological samples based on total RNA according to well-established methods. Briefly, a typical method involves isolating total RNA from the biological sample, amplifying the RNA, synthesizing cDNA, labeling the cDNA with a detectable label, hybridizing the cDNA with a genomic array, such as the Affymetrix U133 GeneChip, and determining binding of the labeled cDNA with the genomic array by measuring the intensity of the signal from the detectable label bound to the array. See, e.g., the methods described in Lu, et al., Chen, et al. and Golub, et al., supra, and the references cited therein, which are incorporated herein by reference. The resulting expression data are input into a database.

mRNAs in the tissue samples can be analyzed using commercially available or customized probes or oligonucleotide arrays, such as cDNA or oligonucleotide arrays. The use of these arrays allows for the measurement of steady-state mRNA levels of thousands of genes simultaneously, thereby presenting a powerful tool for identifying effects such as the onset, arrest or modulation of uncontrolled cell proliferation. Hybridization and/or binding of the probes on the arrays to the nucleic acids of interest from the cells can be determined by detecting and/or measuring the location and intensity of the signal received from the labeled probe or used to detect a DNA/RNA sequence from the sample that hybridizes to a nucleic acid sequence at a known location on the microarray. The intensity of the signal is proportional to the quantity of cDNA or mRNA present in the sample tissue. Numerous arrays and techniques are available and useful. Methods for determining gene and/or protein expression in sample tissues are described, for example, in U.S. Pat. No. 6,271,002; U.S. Pat. No. 6,218,122; U.S. Pat. No. 6,218,114; and U.S. Pat. No. 6,004,755; and in Wang et al., J. Clin. Oncol., 22(9):1564-1671 (2004); Golub et al, (supra); and Schena et al., Science, 270:467-470 (1995); all of which are incorporated herein by reference.

The gene analysis aspect may interrogate gene expression as well as insertion/deletion data. As a first step, RNA is isolated from the tissue samples and labeled. Parallel processes are run on the sample to develop two sets of data: (1) over-/under-expression of genes based on mRNA levels; and (2) chromosomal insertion/deletion data. These two sets of data are then correlated by means of an algorithm Over-/under-expression of the genes in each tissue sample are compared to gene expression in the normal (non-cancerous) samples and other control samples, and a subset of genes that are differentially expressed in the cancer tissue is identified. Preferably, levels of up- and down-regulation are distinguished based on fold changes of the intensity measurements of hybridized microarray probes. A difference of about 2.0 fold or greater is preferred for making such distinctions, or a p-value of less than about 0.05. That is, before a gene is said to be differentially expressed in diseased or suspected diseased versus normal cells, the diseased cell is found to yield at least about 2 times greater or less intensity of expression than the normal cells. Generally, the greater the fold difference (or the lower the p-value), the more preferred is the gene for use as a diagnostic or prognostic tool. Genes identified for the gene signatures of the present invention have expression levels that result in the generation of a signal that is distinguishable from those of the normal or non-modulated genes by an amount that exceeds background using clinical laboratory instrumentation.

Statistical values can be used to confidently distinguish modulated from non-modulated genes and noise. Statistical tests can identify the genes most significantly differentially expressed between diverse groups of samples. The Student's t-test is an example of a robust statistical test that can be used to find significant differences between two groups. The lower the p-value, the more compelling the evidence that the gene is showing a difference between the different groups. Nevertheless, since microarrays allow measurement of more than one gene at a time, tens of thousands of statistical tests may be run at one time. Because of this, it is unlikely to observe small p-values just by chance, and adjustments using a Sidak correction or similar step as well as a randomization/permutation experiment can be made. A p-value less than about 0.05 by the t-test is evidence that the expression level of the gene is significantly different. More compelling evidence is a p-value less than about 0.05 after the Sidak correction is factored in. For a large number of samples in each group, a p-value less than about 0.05 after the randomization/permutation test is the most compelling evidence of a significant difference.

Another parameter that can be used to select genes that generate a signal that is greater than that of the non-modulated gene or noise is the measurement of absolute signal difference. Preferably, the signal generated by the differentially expressed genes differs by at least about 20% from those of the normal or non-modulated gene (on an absolute basis). It is even more preferred that such genes produce expression patterns that are at least about 30% different than those of normal or non-modulated genes. For smaller subsets of genes evaluated, such as profiles containing less than 30, less than or about 20 or less than or about 10 genes, the expression patterns may be at least about 40% or at least about 50% different than those of normal or non-modulated genes.

Differential expression analyses can be performed using commercially available arrays, for example, Affymetrix U133 GeneChip® arrays (Affymetrix, Inc.). These arrays have probe sets for the whole human genome immobilized on the chip, and can be used to determine up- and down-regulation of genes in test samples. Other substrates having affixed thereon human genomic DNA or probes capable of detecting expression products, such as those available from Affymetrix, Agilent Technologies, Inc. or Illumina, Inc. also may be used. Currently preferred gene microarrays for use in the present invention include Affymetrix U133 GeneChip® arrays and Agilent Technologies genomic cDNA microarrays. Instruments and reagents for performing gene expression analysis are commercially available. See, e.g., Affymetrix GeneChip® System. The expression data obtained from the analysis then is input into the database.

For chromosomal insertion/deletion analyses, data for the genes of each sample as compared to samples of normal tissue is obtained. The insertion/deletion analysis is generated using an array-based comparative genomic hybridization (“CGH”). Array CGH measures copy-number variations at multiple loci simultaneously, providing an important tool for studying cancer and developmental disorders and for developing diagnostic and therapeutic targets. Microchips for performing array CGH are commercially available, e.g., from Agilent Technologies. The Agilent chip is a chromosomal array which shows the location of genes on the chromosomes and provides additional data for the gene signature. The insertion/deletion data once acquired from this testing is also input into the database.

The analyses are carried out on the same samples from the same patients to generate parallel data. The same chips and sample preparation are used to reduce variability.

The expression of certain genes known as “reference genes” “control genes” or “housekeeping genes” also is determined, preferably at the same time, as a means of ensuring the veracity of the expression profile. Reference genes are genes that are consistently expressed in many tissue types, including cancerous and normal tissues, and thus are useful to normalize gene expression profiles. See, e.g., Silvia et al., BMC Cancer, 6:200 (2006); Lee et al., Genome Research, 12(2):292-297 (2002); Zhang et al., BMC Mol. Biol., 6:4 (2005). Determining the expression of reference genes in parallel with the genes in the unique gene expression profile provides further assurance that the techniques used for determination of the gene expression profile are working properly. The expression data relating to the reference genes also is input into the database. In a currently preferred embodiment, the following genes are used as reference genes: beta-actin (ACTB), glyceraldehyde-3-phosphate dehydrogenase (GAPDH), beta glucoronidase (GUSB) as positive controls while negative controls include large ribosomal protein (RPLPO) and/or transferrin receptor (TRFC). Beta actin may be used as the positive control for IHC.

### Data Correlation for Gene Expression Profiles

The differential expression data and the insertion/deletion data in the database may be correlated with the clinical outcomes information associated with each tissue sample also in the database by means of an algorithm to determine a gene expression profile for determining or predicting progression as well as recurrence of disease and/or disease-related presentations. Various algorithms are available which are useful for correlating the data and identifying the predictive gene signatures. For example, algorithms such as those identified in Xu et al., A Smooth Response Surface Algorithm For Constructing A Gene Regulatory Network, Physiol. Genomics 11:11-20 (2002), the entirety of which is incorporated herein by reference, may be used for the practice of the embodiments disclosed herein.

Another method for identifying gene expression profiles is through the use of optimization algorithms such as the mean variance algorithm widely used in establishing stock portfolios. One such method is described in detail in the patent application US Patent Application Publication No. 2003/0194734. Essentially, the method calls for the establishment of a set of inputs expression as measured by intensity) that will optimize the return (signal that is generated) one receives for using it while minimizing the variability of the return. The algorithm described in Irizarry et al., Nucleic Acids Res., 31:e15 (2003) also may be used. One useful algorithm is the JMP Genomics algorithm available from JMP Software.

The process of selecting gene expression profiles also may include the application of heuristic rules. Such rules are formulated based on biology and an understanding of the technology used to produce clinical results, and are then applied to output from the optimization method. For example, the mean variance method of gene signature identification can be applied to microarray data for a number of genes differentially expressed in subjects with cancer. Output from the method would be an optimized set of genes that could include some genes that are expressed in peripheral blood as well as in diseased tissue. If samples used in the testing method are obtained from peripheral blood and certain genes differentially expressed in instances of cancer could also be differentially expressed in peripheral blood, then a heuristic rule can be applied in which a portfolio is selected from the efficient frontier excluding those that are differentially expressed in peripheral blood. Other cells, tissues or fluids may also be used for the evaluation of differentially expressed genes, proteins or peptides. Of course, the rule can be applied prior to the formation of the efficient frontier by, for example, applying the rule during data pre-selection.

Other heuristic rules can be applied that are not necessarily related to the biology in question. For example, one can apply a rule that only a certain percentage of the portfolio can be represented by a particular gene or group of genes. Commercially available software such as the Wagner software readily accommodates these types of heuristics (Wagner Associates Mean-Variance Optimization Application). This can be useful, for example, when factors other than accuracy and precision have an impact on the desirability of including one or more genes.

As an example, the algorithm may be used for comparing gene expression profiles for various genes (or portfolios) to ascribe prognoses. The expression profiles (whether at the RNA or protein level) of each of the genes comprising the portfolio are fixed in a medium such as a computer readable medium. This can take a number of forms. For example, a table can be established into which the range of signals (e.g., intensity measurements) indicative of disease is input. Actual patient data can then be compared to the values in the table to determine whether the patient samples are normal or diseased. In a more sophisticated embodiment, patterns of the expression signals (e.g., fluorescent intensity) are recorded digitally or graphically. The gene expression patterns from the gene portfolios used in conjunction with patient samples are then compared to the expression patterns. Pattern comparison software can then be used to determine whether the patient samples have a pattern indicative of recurrence of the disease. Of course, these comparisons can also be used to determine whether the patient is not likely to experience disease recurrence. The expression profiles of the samples are then compared to the profile of a control cell. If the sample expression patterns are consistent with the expression pattern for recurrence of cancer then (in the absence of countervailing medical considerations) the patient is treated as one would treat a relapse patient. If the sample expression patterns are consistent with the expression pattern from the normal/control cell then the patient is diagnosed negative for the cancer.

A method for analyzing the gene signatures of a patient to determine prognosis of cancer is through the use of a Cox hazard analysis program. The analysis may be conducted using S-Plus software (commercially available from Insightful Corporation). Using such methods, a gene expression profile is compared to that of a profile that confidently represents relapse (i.e., expression levels for the combination of genes in the profile is indicative of relapse). The Cox hazard model with the established threshold is used to compare the similarity of the two profiles (known relapse versus patient) and then determines whether the patient profile exceeds the threshold. If it does, then the patient is classified as one who will relapse and is accorded treatment such as adjuvant therapy. If the patient profile does not exceed the threshold then they are classified as a non-relapsing patient. Other analytical tools can also be used to answer the same question such as, linear discriminate analysis, logistic regression and neural network approaches. See, e.g., software available from JMP statistical software.

Numerous other well-known methods of pattern recognition are available. The following references provide some examples:

Weighted Voting: Golub, T R., Slonim, D K., Tamaya, P., Huard, C., Gaasenbeek, M., Mesirov, J P., Coller, H., Loh, L., Downing, J R., Caligiuri, M A., Bloomfield, C D., Lander, E S. Molecular classification of cancer: class discovery and class prediction by gene expression monitoring. Science 286:531-537, 1999.

Support Vector Machines: Su, A I., Welsh, J B., Sapinoso, L M., Kern, S G., Dimitrov, P., Lapp, H., Schultz, P G., Powell, S M., Moskaluk, C A., Frierson, H F. Jr., Hampton, G M. Molecular classification of human carcinomas by use of gene expression signatures. Cancer Research 61:7388-93, 2001. Ramaswamy, S., Tamayo, P., Rifkin, R., Mukherjee, S., Yeang, C H., Angelo, M., Ladd, C., Reich, M., Latulippe, E., Mesirov, J P., Poggio, T., Gerald, W., Loda, M., Lander, E S., Gould, T R. Multiclass cancer diagnosis using tumor gene expression signatures Proceedings of the National Academy of Sciences of the USA 98:15149-15154, 2001.

K-nearest Neighbors: Ramaswamy, S., Tamayo, P., Rifkin, R., Mukherjee, S., Yeang, C H., Angelo, M., Ladd, C., Reich, M., Latulippe, E., Mesirov, J P., Poggio, T., Gerald, W., Loda, M., Lander, E S., Gould, T R. Multiclass cancer diagnosis using tumor gene expression signatures Proceedings of the National Academy of Sciences of the USA 98:15149-15154, 2001.

Correlation Coefficients: van't Veer L J, Dai H, van de Vijver M J, He Y D, Hart A, Mao M, Peters H L, van der Kooy K, Marton M J, Witteveen A T, Schreiber G J, Kerkhoven R M, Roberts C, Linsley P S, Bernards R, Friend S H. Gene expression profiling predicts clinical outcome of prostate cancer, Nature. 2002 Jan. 31; 415(6871):530-6.

The gene expression analysis identifies a gene expression profile (GEP) unique to the cancer samples, that is, those genes which are differentially expressed by the cancer cells. This GEP then is validated, for example, using real-time quantitative polymerase chain reaction (RT-qPCR), which may be carried out using commercially available instruments and reagents, such as those available from Applied Biosystems.

### Determination of Protein Expression Profiles

Not all genes expressed by a cell are translated into proteins, therefore, once a GEP has been identified, it may also be desirable to ascertain whether proteins corresponding to some or all of the differentially expressed genes in the GEP also are differentially expressed by the same cells or tissue. Therefore, protein expression profiles (PEPs) are generated from the same suspect tissue control tissues used to identify the GEPs. PEPs also are used to validate the GEP in other individuals, e.g., prostate cancer patients.

The preferred method for generating PEPs according to the present invention is by immunohistochemistry (IHC) analysis. In this method antibodies specific for the proteins in the PEP are used to interrogate tissue samples from individuals of interest. Other methods for identifying PEPs are known, e.g. in situ hybridization (ISH) using protein-specific nucleic acid probes. See, e.g., Hofer et al., Clin. Can. Res., 11(16):5722 (2005); Volm et al., Clin. Exp. Metas., 19(5):385 (2002). Any of these alternative methods also could be used.

For determining the PEPs samples of suspect tissue, metastatic and normal margin prostate tissue are obtained from patients. These are the same samples used for identifying the GEP. The tissue samples as well as the positive and negative control samples are arrayed on tissue microarrays (TMAs) to enable simultaneous analysis. TMAs consist of substrates, such as glass slides, on which up to about 1000 separate tissue samples are assembled in array fashion to allow simultaneous histological analysis. The tissue samples may comprise tissue obtained from preserved biopsy samples, e.g., paraffin-embedded or frozen tissues. Techniques for making tissue microarrays are well-known in the art. See, e.g., Simon et al., BioTechniques, 36(1):98-105 (2004); Kallioniemi et al, WO 99/44062; Kononen et al., Nat. Med., 4:844-847 (1998). In one method, a hollow needle is used to remove tissue cores as small as 0.6 mm in diameter from regions of interest in paraffin embedded tissues. The “regions of interest” are those that have been identified by a pathologist as containing the desired diseased or normal tissue. These tissue cores are then inserted in a recipient paraffin block in a precisely spaced array pattern. Sections from this block are cut using a microtome, mounted on a microscope slide and then analyzed by standard histological analysis. Each microarray block can be cut into approximately 100 to approximately 500 sections, which can be subjected to independent tests.

Proteins in the tissue samples may be analyzed by interrogating the TMAs using protein-specific agents, such as antibodies or nucleic acid probes, such as oligonucleotides or aptamers. Antibodies are preferred for this purpose due to their specificity and availability. The antibodies may be monoclonal or polyclonal antibodies, antibody fragments, and/or various types of synthetic antibodies, including chimeric antibodies, or fragments thereof Antibodies are commercially available from a number of sources (e.g., Abcam, Cell Signaling Technology or Santa Cruz Biotechnology), or may be generated using techniques well-known to those skilled in the art. The antibodies typically are equipped with detectable labels, such as enzymes, chromogens or quantum dots, which permit the antibodies to be detected. The antibodies may be conjugated or tagged directly with a detectable label, or indirectly with one member of a binding pair, of which the other member contains a detectable label. Detection systems for use with are described, for example, in the website of Ventana Medical Systems, Inc. Quantum dots are particularly useful as detectable labels. The use of quantum dots is described, for example, in the following references: Jaiswal et al., Nat. Biotechnol., 21:47-51(2003); Chan et al., Curr. Opin. Biotechnol., 13:40-46 (2002); Chan et al., Science, 281:435-446 (1998).

The use of antibodies to identify proteins of interest in the cells of a tissue, referred to as immunohistochemistry (IHC), is well established. See, e.g., Simon et al., BioTechniques, 36(1):98 (2004); Haedicke et al., BioTechniques, 35(1):164 (2003), which are hereby incorporated by reference. The RIC assay can be automated using commercially available instruments, such as the Benchmark instruments available from Ventana Medical Systems, Inc.

In one embodiment, the TMAs are contacted with antibodies specific for the proteins encoded by the genes identified in the gene expression study as being differentially expressed in patients whose conditions had progressed to prostate cancer in order to determine expression of these proteins in each type of tissue. The antibodies used to interrogate the TMAs are selected based on the genes having the highest level of differential expression.

### GPEP Assays

The present invention further comprises methods and assays for determining or predicting whether a patient's condition is likely to progress to cancer or whether a patient having cancer has a poor prognosis. According to one aspect, a formatted IHC assay can be used for determining if a tissue sample exhibits any of a GEP, PEP or GPEs. The assays may be formulated into kits that include all or some of the materials needed to conduct the analysis, including reagents (antibodies, detectable labels, etc.) and instructions.

Any of the compositions described herein may be comprised in a kit. In a non-limiting example, reagents for the detection of PEPs, GEPs, or GPEPs are included in a kit. In one embodiment, antibodies to one or more of the expression products of the genes of the GPEPs disclosed herein are included. Antibodies may be included to provide concentrations of from about 0.1 μg/mL to about 500 μg/mL, from about 0.1 μg/mL to about 50 μg/mL or from about 1 μg/mL to about 5 μg/mL or any value within the stated ranges. The kit may further include reagents or instructions for creating or synthesizing further probes, labels or capture agents. It may also include one or more buffers, such as a nuclease buffer, transcription buffer, or a hybridization buffer, compounds for preparing a DNA template, cDNA, primers, probes or label, and components for isolating any of the foregoing. Other kits of the invention may include components for making a nucleic acid or peptide array including all reagents, buffers and the like and thus, may include, for example, a solid support.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. However, various combinations of components may be comprised in a vial or similar container. The kits of the present invention also will typically include a means for containing the detection reagents, e.g., nucleic acids or proteins or antibodies, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the invention. The dye may then be resuspended in any suitable solvent, such as DMSO.

Kits may also include components that preserve or maintain the compositions that protect against their degradation. Such kits generally will comprise, in suitable means, distinct containers for each individual reagent or solution.

Certain assay methods of the invention comprises contacting a tissue sample from an individual with a group of antibodies specific for some or all of the genes or proteins of a GPEP, and determining the occurrence of up- or down-regulation of these genes or proteins in the sample. The use of TMAs allows numerous samples, including control samples, to be assayed simultaneously.

The method preferably also includes detecting and/or quantitating control or “reference proteins”. Detecting and/or quantitating the reference proteins in the samples normalizes the results and thus provides further assurance that the assay is working properly. In a currently preferred embodiment, antibodies specific for one or more of the following reference proteins are included: beta-actin (ACTB), glyceraldehyde-3-phosphate dehydrogenase (GAPDH), beta glucoronidase (GUSB) as positive controls while negative controls include large ribosomal protein (RPLPO) and/or transferrin receptor (TRFC). Beta actin may be used as the positive control for IHC.

In one embodiment, the assay and method comprises determining expression only of the overexpressed genes or proteins in a GPEP. The method comprises obtaining a tissue sample from the patient, determining the gene and/or protein expression profile of the sample, and determining from the gene or protein expression profile.

In one embodiment, the assay and method comprises determining expression only of the overexpressed genes or proteins in the GPEP. The method preferably includes at least one reference protein, which may be selected are beta-actin (ACTB), glyceraldehyde-3-phosphate dehydrogenase (GAPDH), beta glucoronidase (GUSB) as positive controls while negative controls include large ribosomal protein (RPLPO) and/or transferrin receptor (TRFC). Beta actin may be used as the positive control for IHC.

The present invention further comprises a kit containing reagents for conducting an IHC analysis of tissue samples or cells from individuals, e.g., patients, including antibodies specific for at least about two of the proteins in a GPEP and for any reference proteins. The antibodies are preferably tagged with means for detecting the binding of the antibodies to the proteins of interest, e.g., detectable labels. Preferred detectable labels include fluorescent compounds or quantum dots; however other types of detectable labels may be used. Detectable labels for antibodies are commercially available, e.g. from Ventana Medical Systems, Inc.

Immunohistochemical methods for detecting and quantitating protein expression in tissue samples are well known. Any method that permits the determination of expression of several different proteins can be used. See. e.g., Signoretti et al., “Her-2-neu Expression and Progression Toward Androgen Independence in Human Prostate Cancer,” J. Natl. Cancer Instit., 92(23):1918-25 (2000); Gu et al., “Prostate stem cell antigen (PSCA) expression increases with high gleason score, advanced stage and bone metastasis in prostate cancer,” Oncogene, 19:1288-96 (2000). Such methods can be efficiently carried out using automated instruments designed for immunohistochemical (IHC) analysis. Instruments for rapidly performing such assays are commercially available, e.g., from Ventana Molecular Discovery Systems or Lab Vision Corporation. Methods according to the present invention using such instruments are carried out according to the manufacturer's instructions.

Protein-specific antibodies for use in such methods or assays are readily available or can be prepared using well-established techniques. Antibodies specific for the proteins in the GPEP disclosed herein can be obtained, for example, from Cell Signaling Technology, Inc, Santa Cruz Biotechnology, Inc. or Abcam.

### Immunoassays

The present invention provides for new assays useful in the diagnosis, prognosis and prediction of prostate cancer and the elucidation of clinical management parameters associated with prostate cancer. The immunoassays of the present invention utilize the anti-FAS polyclonal or monoclonal antibodies described herein to specifically bind to FAS in a biological sample. Any type of immunoassay format may be used, including, without limitation, enzyme immunoassays (EIA, ELISA), radioimmunoassay (RIA), fluoroimmunoassay (FIA), chemiluminescent immunoassay (CLIA), counting immunoassay (CIA), immunohistochemistry (IHC), agglutination, nephelometry, turbidimetry or Western Blot. These and other types of immunoassays are well-known and are described in the literature, for example, in Immunochemistry, Van Oss and Van Regenmortel (Eds), CRC Press, 1994; The Immunoassay Handbook, D. Wild (Ed.), Elsevier Ltd., 2005; and the references disclosed therein.

The preferred assay format for the present invention is the enzyme-linked immunosorbent assay (ELISA) format. ELISA is a highly sensitive technique for detecting and measuring antigens or antibodies in a solution in which the solution is run over a surface to which immobilized antibodies specific to the substance have been attached, and if the substance is present, it will bind to the antibody layer, and its presence is verified and visualized with an application of antibodies that have been tagged or labeled so as to permit detection. ELISAs combine the high specificity of antibodies with the high sensitivity of enzyme assays by using antibodies or antigens coupled to an easily assayed enzyme that possesses a high turnover number such as alkaline phosphatase (AP) or horseradish peroxidase (HRP), and are very useful tools both for determining antibody concentrations (antibody titer) in sera as well as for detecting the presence of antigen.

There are many different types of ELISAs; the most common types include “direct ELISA,” “indirect ELISA,” “sandwich ELISA” and cell-based ELISA (C-ELISA). Performing an ELISA involves at least one antibody with specificity for a particular antigen. The sample with an unknown amount of antigen is immobilized on a solid support (usually a polystyrene microtiter plate) either non-specifically (via adsorption to the surface) or specifically (via capture by another antibody specific to the same antigen, in a “sandwich” ELISA). After the antigen is immobilized the detection antibody is added, forming a complex with the antigen. The detection antibody can be covalently linked to an enzyme, or can itself be detected by a secondary antibody which is linked to an enzyme through bioconjugation. Between each step the plate typically is washed with a mild detergent solution to remove any proteins or antibodies that are not specifically bound. After the final wash step the plate is developed by adding an enzymatic substrate tagged with a detectable label to produce a visible signal, which indicates the quantity of antigen in the sample.

In a typical microtiter plate sandwich immunoassay, an antibody (“capture antibody”) is adsorbed or immobilized onto a substrate, such as a microtiter plate. Monoclonal antibodies are preferred as capture antibodies due to their greater specificity, but polyclonal antibodies also may be used. When the test sample is added to the plate, the antibody on the plate will bind the target antigen from the sample, and retain it in the plate. When a second antibody (“detection antibody”) or antibody pair is added in the next step, it also binds to the target antigen (already bound to the monoclonal antibody on the plate), thereby forming an antigen ‘sandwich’ between the two different antibodies.

This binding reaction can then be measured by radio-isotopes, as in a radio-immunoassay format (RIA); by enzymes, as in an enzyme immunoassay format (EIA or ELISA); or other detectable label, attached to the detection antibody. The label generates a color signal proportional to the amount of target antigen present in the original sample added to the plate. Depending on the immunoassay format, the degree of color can be detected and measured with the naked eye (as with a home pregnancy test), a scintillation counter (for an RIA), or with a spectrophotometric plate reader (for an EIA or ELISA).

The assay then is carried out according to the following general steps:

Step 1: Capture antibodies are adsorbed onto the well of a plastic microtiter plate (no sample added);

Step 2: A test sample (such as human serum) is added to the well of the plate, under conditions sufficient to permit binding of the target antigen to the capture antibody already bound to the plate, thereby retaining the antigen in the well;

Step 3: Binding of a detection antibody or antibody pair (with enzyme or other detectable moiety attached) to the target antigen (already bound to the capture antibody on the plate), thereby forming an antigen “sandwich” between the two different antibodies. The detectable label on the detection antibodies will generate a color signal proportional to the amount of target antigen present in the original sample added to the plate.

In an alternative embodiment, sometimes referred to as an antigen-down immunoassay, the analyte (rather than an antibody) is coated onto a substrate, such as a microtiter plate, and used to bind antibodies found in a sample. When the sample is added (such as human serum), the antigen on the plate is bound by antibodies (IgE for example) from the sample, which are then retained in the well. A species-specific antibody (anti-human IgE for example) labeled with an enzyme such as horse radish peroxidase (HRP) is added next, which, binds to the antibody bound to the antigen on the plate. The higher the signal, the more antibodies there are in the sample.

In another embodiment, an immunoassay may be structured in a competitive inhibition format. Competitive inhibition assays are often used to measure small analytes because competitive inhibition assays only require the binding of one antibody rather than two as is used in standard ELISA formats. In a sequential competitive inhibition assay, the sample and conjugated analyte are added in steps similar to a sandwich assay, while in a classic competitive inhibition assay, these reagents are incubated together at the same time.

In a typical sequential competitive inhibition assay format, a capture antibody is coated onto a substrate, such as a microtiter plate. When the sample is added, the capture antibody captures free analyte out of the sample. In the next step, a known amount of analyte labeled with a detectable label, such as an enzyme or enzyme substrate, added. The labeled analyte also attempts to bind to the capture antibody adsorbed onto the plate, however, the labeled analyte is inhibited from binding to the capture antibody by the presence of previously bound analyte from the sample. This means that the labeled analyte will not be bound by the monoclonal on the plate if the monoclonal has already bound unlabeled analyte from the sample. The amount of unlabeled analyte in the sample is inversely proportional to the signal generated by the labeled analyte. The lower the signal, the more unlabeled analyte there is in the sample. A standard curve can be constructed using serial dilutions of an unlabeled analyte standard. Subsequent sample values can then be read off the standard curve as is done in the sandwich ELISA formats. The classic competitive inhibition assay format requires the simultaneous addition of labeled (conjugated analyte) and unlabeled analyte (from the sample). Both labeled and unlabeled analyte then compete simultaneously for the binding site on the monoclonal capture antibody on the plate. Like the sequential competitive inhibition format, the colored signal is inversely proportional to the concentration of unlabeled target analyte in the sample. Detection of labeled analyte can be read on a microtiter plate reader.

In addition to microtiter plates, immunoassays are also may be configured as rapid tests, such as a home pregnancy test. Like microtiter plate assays, rapid tests use antibodies to react with antigens and can be developed as sandwich formats, competitive inhibition formats, and antigen-down formats. With a rapid test, the antibody and antigen reagents are bound to porous membranes, which react with positive samples while channeling excess fluids to a non-reactive part of the membrane. Rapid immunoassays commonly come in two configurations: a lateral flow test where the sample is simply placed in a well and the results are read immediately; and a flow through system, which requires placing the sample in a well, washing the well, and then finally adding an analyte-detectable label conjugate and the result is read after a few minutes. One sample is tested per strip or cassette. Rapid tests are faster than microtiter plate assays, require little sample processing, are often cheaper, and generate yes/no answers without using an instrument. However, rapid immunoassays are not as sensitive as plate-based immunoassays, nor can they be used to accurately quantitate an analyte.

The preferred technique for use in the present invention to detect the amount of FAS in circulating cells is the sandwich ELISA, in which highly specific monoclonal antibodies are used to detect sample antigen. The sandwich ELISA method comprises the following general steps:

1. Prepare a surface to which a known quantity of capture antibody is bound;

2. (Optionally) block any non specific binding sites on the surface;

3. Apply the antigen-containing sample to the surface;

4. Wash the surface, so that unbound antigen is removed;

5. Apply primary (detection) antibodies that bind specifically to the bound antigen;

6. Apply enzyme-linked secondary antibodies which are specific to the primary antibodies;

7. Wash the plate, so that the unbound antibody-enzyme conjugates are removed;

8. Apply a chemical which is converted by the enzyme into a detectable (e.g., color or fluorescent or electrochemical) signal; and

9. Measure the absorbance or fluorescence or electrochemical signal to determine the presence and quantity of antigen.

In an alternate embodiment, the primary antibody (step 5) is linked to an enzyme; in this embodiment, the use of a secondary antibody conjugated to an enzyme (step 6) is not necessary if the primary antibody is conjugated to an enzyme. However, use of a secondary-antibody conjugate avoids the expensive process of creating enzyme-linked antibodies for every antigen one might want to detect. By using an enzyme-linked antibody that binds the Fc region of other antibodies, this same enzyme-linked antibody can be used in a variety of situations. The major advantage of a sandwich ELISA is the ability to use crude or impure samples and still selectively bind any antigen that may be present. Without the first layer of “capture” antibody, any proteins in the sample (including serum proteins) may competitively adsorb to the plate surface, lowering the quantity of antigen immobilized.

In one embodiment of the present invention, a solid phase substrate, such as a microtiter plate or strip, is treated in order to fix or immobilize a capture antibody to the surface of the substrate. The material of the solid phase is not particularly limited as long as it is a material of a usual solid phase used in immunoassays. Examples of such material include polymer materials such as latex, rubber, polyethylene, polypropylene, polystyrene, a styrene-butadiene copolymer, polyvinyl chloride, polyvinyl acetate, polyacrylamide, polymethacrylate, a styrene-methacrylate copolymer, polyglycidyl methacrylate, an acrolein-ethyleneglycol dimethacrylate copolymer, polyvinylidene difluoride (PVDF), and silicone; agarose; gelatin; red blood cells; and inorganic materials such as silica gel, glass, inert alumina, and magnetic substances. These materials may be used singly or in combination of two or more thereof

The form of the solid phase is not particularly limited insofar as the solid phase is in the form of a usual solid phase used in immunoassays, for example in the form of a microtiter plate, a test tube, beads, particles, and nanoparticles. The particles include magnetic particles, hydrophobic particles such as polystyrene latex, copolymer latex particles having hydrophilic groups such as an amino group and a carboxyl group on the surfaces of the particles, red blood cells and gelatin particles. The solid phase is preferably a microtiter plate or strip, such as those available from Cell Signaling Technology, Inc.

The capture antibody preferably is one or more monoclonal anti-FAS antibodies described herein that specifically bind to at least a portion of one or more of the peptide sequences of SEQ ID NO. 1-5. Where microtiter plates or strips are used, the capture antibody is immobilized within the wells. Techniques for coating and/or immobilizing proteins to solid phase substrates are known in the art, and can be achieved, for example, by a physical adsorption method, a covalent bonding method, an ionic bonding method, or a combination thereof See, e.g., W. Luttmann et al., Immunology, Ch. 4.3.1 (pp. 92-94), Elsevier, Inc. (2006) and the references cited therein. For example, when the binding substance is avidin or streptavidin, a solid phase to which biotin was bound can be used to fix avidin or streptavidin to the solid phase. The amounts of the capture antibody, the detection antibody and the solid phase to be used can also be suitably established depending on the antigen to be measured, the antibody to be used, and the type of the solid phase or the like. Protocols for coating microtiter plates with capture antibodies, including tools and methods for calculating the quantity of capture antibody, are described for example, on the websites for Immunochemistry Technologies, LLC (Bloomington, Minn.) and Meso Scale Diagnostics, LLC (Gaithersburg, Md.).

The detection antibody can be any anti-FAS antibody. Anti-FAS antibodies are commercially available, for example, from Cell Signaling Technologies, Inc., Santa Cruz Biotechnology, EMD Biosciences, and others. The detection antibody also may be an anti-FAS antibody as disclosed herein that is specific for one or more of SEQ ID NOs. 1-5. In one embodiment, the detection antibody may be directly conjugated with a detectable label, or an enzyme. If the detection antibody is not conjugated with a detectable label or an enzyme, then a labeled secondary antibody that specifically binds to the detection antibody is included. Such detection antibody “pairs” are commercially available, for example, from Cell Signaling Technologies, Inc.

Techniques for labeling antibodies with detectable labels are well-established in the art. As used herein, the term “detectable label” refers to a composition detectable by spectroscopic, photochemical, biochemical, immunochemical, or chemical means. The detectable label can be selected, e.g., from a group consisting of radioisotopes, fluorescent compounds, chemiluminescent compounds, enzymes, and enzyme co-factors, or any other labels known in the art. See, e.g., Zola, Monoclonal Antibodies: A Manual of Techniques, pp. 147-158 (CRC Press, Inc. 1987). A detectable label can be attached to the subject antibodies and is selected so as to meet the needs of various uses of the method which are often dictated by the availability of assay equipment and compatible immunoassay procedures. Appropriate labels include, without limitation, radionuclides, enzymes (e.g., alkaline phosphatase, horseradish peroxidase, luciferase, or β-galactosidase), fluorescent moieties or proteins (e.g., fluorescein, rhodamine, phycoerythrin, GFP, or BFP), or luminescent moieties (e.g., Evidot® quantum dots supplied by Evident Technologies, Troy, N.Y., or Qdot™ nanoparticles supplied by the Quantum Dot Corporation, Palo Alto, Calif).

Preferably, the sandwich immunoassay of the present invention comprises the step of measuring the labeled secondary antibody, which is bound to the detection antibody, after formation of the capture antibody-antigen-detection antibody complex on the solid phase. The method of measuring the labeling substance can be appropriately selected depending on the type of the labeling substance. For example, when the labeling substance is a radioisotope, a method of measuring radioactivity by using a conventionally known apparatus such as a scintillation counter can be used. When the labeling substance is a fluorescent substance, a method of measuring fluorescence by using a conventionally known apparatus such as a luminometer can be used.

When the labeling substance is an enzyme, a method of measuring luminescence or coloration by reacting an enzyme substrate with the enzyme can be used. The substrate that can be used for the enzyme includes a conventionally known luminescent substrate, calorimetric substrate, or the like. When an alkaline phosphatase is used as the enzyme, its substrate includes chemilumigenic substrates such as CDP-star® (4-chloro-3-(methoxyspiro(1,2-dioxetane-3,2′-(5′-chloro)tricyclo[3.3.1.1.-sup.3.7]decane)-4-yl)disodium phenylphosphate) and CSPD® (3-(4-methoxyspiro(1,2-dioxetane-3,2-(5′-chloro)tricyclo[3.3.1.1.sup.3.7]-decane)-4-yl)disodium phenylphosphate) and colorimetric substrates such as p-nitrophenyl phosphate, 5-bromo-4-chloro-3-indolyl-phosphoric acid (BCIP), 4-nitro blue tetrazolium chloride (NBT), and iodonitro tetrazolium (NT). These luminescent or calorimetric substrates can be detected by a conventionally known spectrophotometer, luminometer, or the like.

In one embodiment, the detectable labels comprise quantum dots (e.g., Evidot® quantum dots supplied by Evident Technologies, Troy, N.Y., or Qdot™ nanoparticles supplied by the Quantum Dot Corporation, Palo Alto, Calif.). Techniques for labeling proteins, including antibodies, with quantum dots are known. See, e.g., Goldman et al., Phys. Stat. Sol., 229(1): 407-414 (2002); Zdobnova et al., J. Biomed. Opt., 14(2):021004 (2009); Lao et al., JACS, 128(46):14756-14757 (2006); Mattoussi et al., JACS, 122(49):12142-12150 (2000); and Mason et al., Methods in Molecular Biology: NanoBiotechnology Protocols, 303:35-50 (Springer Protocols, 2005). Quantum-dot antibody labeling kits are commercially available, e.g., from Invitrogen (Carlsbad, Calif.) and Millipore (Billerica, Mass.).

The sandwich immunoassay of the present invention may comprise one or more washing steps. By washing, the unreacted reagents can be removed. For example, when the solid phase comprises a strip of microtiter wells, a washing substance or buffer is contacted with the wells after each step. Examples of the washing substance that can be used include 2-[N-morpholino]ethanesulfonate buffer (MES), or phosphate buffered saline (PBS), etc. The pH of the buffer is preferably from about pH 6.0 to about pH 10.0. The buffer may contain a detergent or surfactant, such as Tween 20.

The sandwich immunoassay can be carried out under typical conditions for immunoassays. The typical conditions for immunoassays comprise those conditions under which the pH is about 6.0 to 10.0 and the temperature is about 30 to 45° C. The pH can be regulated with a buffer, such as phosphate buffered saline (PBS), a triethanolamine hydrochloride buffer (TEA), a Tris-HCl buffer or the like. The buffer may contain components used in usual immunoassays, such as a surfactant, a preservative and serum proteins. The time of contacting the respective components in each of the respective steps can be suitably established depending on the antigen to be measured, the antibody to be used, and the type of the solid phase or the like.

### Kits

The materials for use in the methods of the present invention are suited for preparation of kits produced in accordance with well known procedures. The invention thus provides kits comprising agents, which may include gene-specific or gene-selective probes and/or primers, for quantitating the expression of the disclosed genes for predicting prognostic outcome or response to treatment. Such kits may optionally contain reagents for the extraction of RNA from tumor samples, in particular fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microtiter plates suitable for use in an automated implementation of the method), each with one or more of the various reagents (typically in concentrated form) utilized in the methods, including, for example, pre-fabricated microarrays, buffers, and the like.

The methods provided by the present invention may also be automated in whole or in part. The invention further provides kits for performing an immunoassay using the FAS antibodies of the present invention.

All aspects of the present invention may also be practiced such that a limited number of additional genes that are co-expressed with the disclosed genes (e.g., one or more genes from the GPEPs or FAS), for example as evidenced by high Pearson correlation coefficients, are included in a prognostic or predictive tests in addition to and/or in place of disclosed genes.

The invention is further illustrated by the following non-limiting examples.

### EXAMPLES

### Example 1

### Gene ExpressionProfile (GEP) Analysis

Gene expression profiles of post-surgical tumor collections were generated for 2351 patients in clinical study (NU9900), and 2911 patients in clinical study (NU9901) with prostate adenocarcinomas. Expression data from the two studies were normalized together by Robust Microarray Analysis (RMA). The adenocarcinoma measure used for all analyses was pathological (Cancer)(PS-pCA) in prostate tissue based on central review of biopsies within 12 months of the initial disease detection. Metrics associated with the two clinical study subsets are shown in Table 3.

TABLE 3
Comparison of two clinical study subsets

| | | Study Identifier |
|---|---|---|
| | Study Identifier | (NUC9901) |
| | (NUC9900) | Prostate |
| | Prostate Adenocarcinoma | Adenocarcinoma |
| | Gleason Grade 5-7 | Gleason Grade 5-7 |
| Gene/Protein/Serum | Yes | Yes |
| biomarker based | | |
| determination | | |
| Patient Setting | Inpatient | Inpatient |
| Number of Patients | 2351 | 2911 |
| Post-Surgical Tumor | Yes | Yes |
| Collection | | |
| Number of patients with | 2351 | 2911 |
| PS-pCA total in Prostate | | |
| Gene array type | Affymetrix HU133A-B | Affymetrix |
| | | HU133A-B |

Gene expression data from the two studies was obtained via immunohistochemical methodology whereby biopsy tissue samples were obtained from patients with adenocarcinomas. Control samples were also obtained. Gene expression profiles (GEPs) then were generated from the biological samples based on total RNA according to well-established methods (See Affymetrix GeneChip expression analysis technical manual, Affymetrix, Inc, Santa Clara, Calif.). Briefly, total RNA was isolated from the biological sample, amplified and cDNA synthesized. cDNA was then labeled with a detectable label, hybridized with a the Affymetrix U133 GeneChip genomic array, and binding of the cDNA to the array was quantified by measuring the intensity of the signal from the detectable cDNA label bound to the array.

### Example 2

### Identification of Single Gene Markers

Gene Ontology (GO) analysis was used as described by Lee H K et al., 2005, “Tool for functional analysis of gene expression data sets,” BMC Bioinformatics, 6: 269; (See also: The Gene Ontology Consortium. “Gene ontology: tool for the unification of biology.” Nat. Genet. May 2000; 25(1):25-9 at http://www.geneontology.org) with 10,000 iterations of the Gene Score Re-sampling Algorithm. A gene network was built using the GeneGo program. Initial analyses used all detection of adenocarcinomas.

### Example 3

### Multi-Probe-Set Predictive Models

To develop a predictive GPEP (gene-protein expression profile), 21,485 probe sets were filtered by removing (a) probe sets with low expression over all samples; and (b) probe sets with low variance over all samples. This yielded 12,385 probe sets for subsequent analyses. Normalized log2(intensity) values were centered by subtracting the study-specific mean for each probe set, and resealed by dividing by the pooled within-study standard deviation for each probe set.

A two-stage model-building approach was used to arrive at the best predictive model.

### Single-Gene Markers

Single-probe-set analyses for dimension reduction were performed. This analysis involves an initial search for probe sets that showed a difference between the two studies in the relationship between expression level and response status, by either logistic regression or linear regression. This yielded 609 probe sets.

### Multi-Gene Markers

A fit was examined with multi-probe-set predictive models. Here, the pre-selected probe sets from the single-probe-set analyses of Gleason grade 5-7 were used as the starting point. Then the initial predictive models to each study were fit separately using a threshold gradient descent (TGD) method for regularized classification. Recursive feature elimination (RFE) was applied to attempt to simplify the models without appreciable loss of predictive accuracy.

The model selection criterion was the mean area under the ROC curve (AUC) from 50 replicates of a 4-fold cross-validation. Then from each RFE model series, here, one per study, the model with maximum difference between the selection criteria for the two studies was selected. The TGD method also was used to build predictive models based on expression of two individual probe sets.

### Example 4

### Predictors of Metastasis

The predictive capacity of measurements of PSA (prostate specific antigen), FAS and FAS/PSA combination were evaluated and a detection rates determined The rates were determined for (a) each condition for all prostate cancer patients (Gleason Grade 5-7), and (b) for only patients with estimated detection probability>an arbitrary threshold of 0.5 based on PSA alone, FAS/PSA combination or FAS alone expression level. The results of the analyses are shown in Table 4. It is evident from the data that the use of FAS as a disease biomarker shows power when combined with or without PSA. It can be seen from the table that PSA alone is a poor predictor of metastasis detection. In contrast, FAS alone was an excellent predictor of metastasis and early metastasis detection.

TABLE 4
FAS as a predictor of metastasis

| | | Study Identifier | | | Study Identifier | | |
|---|---|---|---|---|---|---|---|
| | | (NUC9900) | | | (NUC9901) | | |
| | | | | Detection | | | Detection |
| Model | Subset | R | N | Rate | R | N | Rate |
| PSA alone | Post | 1865 | 2351 | 0.79 | 2439 | 2911 | 0.83 |
| | Surgical | | | | | | |
| | evaluation- | | | | | | |
| | Prediction of | | | | | | |
| | metastasis | | | | | | |
| FAS/PSA | Post | 2196 | 2351 | 0.93 | 2719 | 2911 | 0.93 |
| combination | Surgical | | | | | | |
| | evaluation- | | | | | | |
| | Prediction of | | | | | | |
| | metastasis | | | | | | |
| FAS alone | Post | 2231 | 2351 | 0.94 | 2799 | 2911 | 0.95 |
| | Surgical | | | | | | |
| | evaluation- | | | | | | |
| | Prediction of | | | | | | |
| | metastasis | | | | | | |

R = True number of detections of metastatic disease,
N = Total number of patients in subset,
Detection Rate = R/N.

### Example 5

### Prediction of Aggressive Changes in Post Surgical Prostate Cancer: Univariate and Multivariate Analysis

A series of prognostic factors including primary tumor size, Gleason grade 5-7, histologic grade, FAS status by immunohistochemistry (IHC) and androgen status were tested for the prediction of early recurrence (ERec), late recurrence (LRec) and disease related death (DRD) in post-surgical prostate cancer (PS-pCA) patients.

The study involved the evaluation of formalin fixed paraffin embedded primary PS-pCA specimens from 3261 men (median age 65 years) followed for a minimum of 120 months (10 years). The specimens collected were evaluated for primary tumor size, Gleason grade 5-7, histologic grade, FAS status by immunohistochemistry (IHC) and androgen status. In this study, IHC and ELISA assay were performed using a commercial anti-FAS antibody. For IHC the antibodies used were human anti-FASN Antibody, Affinity Purified (Catalog No. A301-324A) from Bethyl Laboratories (Montgomery, Tex.). For ELISA studies, the antibodies used were the Fatty Acid Synthase Antibody Pair (Catalog No. H00002194-AP11) from Novus Biologicals (Littleton, Colo.). The pair contains a Capture antibody which is rabbit affinity purified polyclonal anti-FASN (100 ug) and a Detection antibody which is mouse monoclonal anti-FASN, IgG1 Kappa (20 ug). No patients received adjuvant treatment prior of the first episode of disease recurrence.

On univariate analysis FAS expression levels by IHC independently predicted ERec (early recurrence) (p<0.0002) LRec (late recurrence) (p<0.0005); and DRD (disease related death) (p<0.0003).

When these data were stratified into FAS expression levels of (a) non-expression represented by an expression level of less than 1% relative to a control (where control is normal tissue expression of FAS which was set as a zero point), (b) borderline expression represented by an expression level of less than 25% over the normal control and (c) highly expressed represented by an expression level of greater than 50% over the normal control, patients in group (c) with highly expressed tumors had a relative risk (adjusted relative hazard) of ERec of 9.4 (range 4.8-22.4): LRec of 6.4 (range 2.1-13.0) and DRD of 19.2 (range 5.0-33.8). A relative risk or adjusted relative hazard is the ratio of the probability of the event occurring in the exposed group versus a non-exposed group.

Tumor size, histologic grade, Gleason grade and androgen receptor status did not consistently predict ERec, LRec or DRD. On multivariate analysis FAS expression levels by IHC predicted ERec, LRec, and DRD independent of tumor size, grade, and androgen receptor status.

Consequently, the data indicate that in this series of PS-pCA patients, FAS expression levels by IHC significantly predicted early and late disease recurrence and disease related death independent of tumor size, grades, and androgen receptor status. It was therefore concluded that the basic FAS Immunohistochemistry (Ventana)/ELISA based serum assay, and optionally including application of the algorithm disclosed herein to interpret the assay, would serve as a reliable prognostic tool and a perfect companion diagnostic to justify additional imaging related studies such as secondary PET, CT, ultrasound or Prostate MRI.

### Example 6

### Preparation of Anti-FAS Monoclonal Antibodies

Anti-FAS antibodies and an immunohistochemical ELISA assay employing the antibodies are disclosed in PCT Publication PCT/US2010/030545 published Oct. 14, 2010, and PCT/US2010/046773 published Mar. 17, 2011, respectively. The contents of each are incorporated here by reference in their entirety.

Briefly, four murine monoclonal antibodies were prepared by immunizing SCID mice with synthetic FAS peptides, and establishing hybridomas according to the general procedure described by Iyer et al., Ind. J. Med. Res., 123:651-564 (2006). Each mouse was immunized with one peptide of SEQ ID NOs 1-5.

Humanized monoclonal antibodies were prepared as described by Carter et al., Proc. Natl. Acad. Sci. USA, 89:4285-89 (1992) from monoclonal antibodies derived from hybridomas A,B, D and E. The humanized monoclonal antibodies (MAbs) are referred to hereinafter as FAS 1, FAS 2, FAS 4 (ATCC Deposit No: PTA-10811) and FAS 5, respectively.

### Example 7

### ELISA Protocol for Chemiluminescence

BLACK wells were coated with 100 μl/well of coating antibody diluted in appropriate buffer (PBS/PBS-T (0.05% Tween20)). Plates were then incubated overnight at 4° C., covered with plate sealer. The plates were then washed with 300 ul of 5× PBS-T on a Wellwash Versa Plate washer (Thermo). The plates were then blocked with ELISA Blocker Blocking Solution (300 μl/well) (Thermo) for 2 hr at 23° C. with shaking at 100 rpm in Incubating Microplate Shaker (VWR) covered with a plate sealer. Afterwards, plates were washed with 5× PBS-T (300 μl/well) on a plate washer. After washing, the plates were tapped on a kimwipe placed on the bench to remove excess liquid.

Standards were prepared in advance and included a 7-point dilution (e.g. in 1% BSA in PBS-T from 500 pg/ml). Once prepared, 100 μl of standards or samples freshly diluted in appropriate buffer (PBS-T, R&D Diluent 7, 18 etc.) are loaded at 23° C. on plate shaker with 100 rpm agitation for 2 hr while covered with plate sealer. Plates were then washed with 5× PBS-T (300 ul/well) on a plate washer.

The detection antibody (100 μl/well; diluted in buffer to appropriate concentration, e.g., in PTS/PBS-T) is incubated for 2 hours at 23° C. on a plate shaker with 100 rpm agitation covered with a plate sealer. The plates were then washed with 5× with PBS-T (300 ul/well) on a plate washer.

The secondary antibody (100 μl/well of appropriate secondary antibody streptavidin-HRP, 1:200 dilution in PBS) is incubated at 23° C. on plate shaker with 100 rpm agitation for 20 min covered with a plate sealer. Alternatively anti-species-HRP antibody at 1:10,000 in PBS for 1 hr at 23° C. on plate shaker with 100 rpm agitation was used. The plates were then washed with 5× PBS-T (300 ul/well) on a plate washer.

The signal was amplified by adding 100 μl/well R&D Gloset Substrate, for 10 min at room temperature in a BioTek FL800x plate reader.

Substrates, which were prepared fresh ahead of time are made by mixing Reagent A (stabilized enhanced luminal) with Reagent B (stabilized hydrogen peroxide) in a 1:2 ratio.

The signal was measured on a BioTek FL800x fluorometer (0.5 s read time) with sensitivity auto-adjusted to the highest point on a standard curve and set to a reading of 100,000.

It should be noted that ELISA Sandwich assays useful in the present invention include those as described in PCT Publication PCT/US2010/046773 published Mar. 17, 2011, the contents of which are incorporated here by reference in its entirety.

### Example 8

### Sample Preparation and In Situ Hybridization Protocols

### A. FFPE Pretreatment Protocol for FISH

The purpose of FFPE pretreatment is to prepare formalin fixed paraffin-embedded (FFPE) tissue sections fixed on positively charged slides for use in fluorescence in situ hybridization (FISH) with CEP and LSI DNA FISH probes. The procedure has been designed to maximize tissue permeability for FISH when using DNA FISH probes.

Specimen [000258] Formalin fixed paraffin-embedded (FFPE) tissue specimens prepared on microscope slides.

### Reagents and Instrumentation

Preparation involved the use of reagents Provided In Kit (Cat #32-801210). Not provided in the kit are: absolute ethanol (EtoH), Hemo-De Clearing Agent (Scientific Safety Solvents Cat. #HD-150), purified water (distilled or deionized), Coplin jars (16 slides/8 slots capacity maximum), 37° C. and 80° C. water baths (one at 73° C. for the probe assay).

### Paraffin Pretreatment Procedure

Sample Slides Preparation: Samples used are fixed in formalin for between 24-48 hours.

1. Cut 4-5 μm thick paraffin sections using a microtome.

2. Float the sections on a purified (i.e., triple distilled) water bath at 40° C.

3. Mount a section on a positively charged slide.

4. Air dry the slides.

5. Bake the slides overnight at 56° C.

### Deparaffinizing Slides

1. Immerse slides in Hemo-De for 5 minutes at ambient temperature.

2. Repeat step one (1) twice using fresh Hemo-De each time.

3. Dehydrate slides in 100% EtOH for 1 minute at ambient temperature. Repeat.

4. Air dry slides for 2-5 minutes, if desired.

### Slide Pretreatment

1. Immerse slides in Pretreatment Solution at 80° C. for 10 minutes. If necessary, two slides may be placed back-to-back in each slot in the Coplin jar, with one slide placed in each end slot. For the end slides, the side of the slide with the tissue section must face the center of the jar.

2. Immerse slides in purified water for 3 minutes.

### Protease Pretreatment

1. Remove slides from the jar of purified water.

2. Remove excess water by blotting the edges of the slides on a paper towel.

3. Immerse slides in Protease solution at 37° C. for 15 minutes. (Ensure that the temperature of the buffer is 37±1° C. prior to adding 250 mg (one tube) protease. If necessary, two slides may be placed back-to-back in each slot in the Coplin jar, with one slide placed in each end slot. For the end slides, the side of the slide with the tissue section must face the center of the jar.

4. Immerse slides in purified water for 3 minutes.

5. Air dry slides for 2-5 minutes.

### Fixing the Sample (Optional)

Fixation of the sample is performed to minimize tissue loss during sample denaturation. This procedure is highly recommended when processing samples in a denaturation bath format, but is not necessary when processing slides using a Co-denaturation/Hybridization protocol.

1. Fill one (1) Coplin jar with 50 mL of 10% buffered formalin. Fill three (3) other Coplin jars with 50 mL of 70% ethanol, 85% ethanol and 100% ethanol in each.

2. Immerse the slides in 10% buffered formalin at ambient temperature for 10 minutes.

3. Immerse the slides in purified water for 3 minutes.

4. Air dry slides.

5. Proceed with the appropriate probe protocol.

### B. Preparation of Metaphase Chromosome Spreads on Microscope Slides for FISH/ISH Analysis

The purpose of this procedure is to prepare human metaphase chromosome spreads and interphase nuclei on microscope slides for cytogenetic analysis and to prepare chromosome preparations for FISH/ISH hybridization procedures.

### Specimen

PHA-stimulated human lymphocytes in 3:1 methanol:glacial acetic acid fixative. The specimens are prepared as described below under “Preparation of Peripheral Blood Cells for Chromosome Analysis”.

### Reagents and Instrumentation

| Item | Supplier | Catalog No. |
|---|---|---|
| Acetic acid, glacial, 500 mL | VWR | JT9511-5 |
| Methanol, 1.0 L | VWR | JT9049-2 |
| Benchtop Centrifuge, 4x 100 mL capacity | VWR | 53513-800 |
| BD Falcon Centrifuge Tubes, conical, 15 mL | VWR | |
| VWR Superfrost Plus Micro slides | VWR | 48311-703 |
| Glass Pasteur Pipettes with bulb or P-1000 μl | | |
| pipette | | |
| Rectangular Staining Dish With Glass Cover | | |
| Distilled Water | | |
| Kimwipes | | |
| Paper Towels | | |
| Phase Contrast Microscope | | |
| Ethanol Series, 70%, 85%, 100% | | |
| 20 X SSC stock for 2 X SSC | | |
| 37° C. water bath | | |
| PHA-stimulated lymphocyte cell pellet | | |

### Preparation

Fixative: Methanol:glacial acetic acid, 3:1. Prepare before each use.

Slides: Label each Superfrost Plus slide accordingly on its frosted surface and place the slides in a rectangular staining dish with glass cover. Fill the dish with distilled water and soak at 4° C. prior to use to chill slides. This can be done days in advance, and slides can be stored at 4° C.

Humidity: Recommended ambient conditions are 25° C. and 33% humidity.

PHA-stimulated Lymphocyte Cell Pellet: Prepare the PHA-stimulated lymphocyte cell pellet in fresh fixative in a 15 mL conical tube. If the pellet was stored after its harvest, centrifuge it at 200×g for 5 minutes. Aspirate the supernatant, and add sufficient fixative to make the cell suspension appear slightly cloudy. Cell concentration varies between cases and should be empirically determined.

Ethanol Series: Prepare v/v dilutions of 100% ethanol with purified H₂O. Between uses, store tightly covered at ambient temperature. Discard stock solutions after 6 months. Prepare 70%, 85% and 100% ethanol using distilled water in plastic Coplin jars.

2×SSC: Mix thoroughly 100 mL 20×SSC (pH 5.3) with 850 mL purified H₂O. Measure pH and adjust to pH 7.0±0.2 with NaOH. Add purified H₂O to bring final volume to 1 liter. Store at ambient temperature. Discard stock solution after 6 months, or sooner if solution appears cloudy or contaminated. Prepare 2×SSC in plastic coplin jar and preheat to 37° C. using a water bath.

### Procedure

Dropping the cell suspension on slides.

1. Remove the staining dish containing the Superfrost Plus microscope slides from the 4° C. storage and place on the lab bench.

2. Using a glass Pasteur pipette with bulb (or P-1000 pipette), gently resuspend the cell pellet in the fixative and set aside in a tube rack.

3. Remove one microscope slide from the chilled staining jar, holding it by the frosted end. Allow the water to drain from the slide so that a thin film of water remains on the slide surface.

4. Resuspend the cell pellet using the P-1000 pipette with appropriate tip and then draw 300 ul of the cell suspension.

5. Holding the slide at an angle (˜45° expel the cell suspension down the length of the slide, starting at the frosted end. Move the pipette tip across the surface of the slide just below the frosted area from one edge to the other as the suspension is expelled.

6. Drain the excess cell suspension and fixative from the slide by touching the edges of the slide on a dry paper towel.

7. Position the slide at an ˜45° C. angle with the cell sample side facing up to dry and allow the fixative to evaporate.

8. Review the slide preparation with Phase Contrast Microscopy. (See Notes 1-4, below)

9a. Continue to prepare slides as needed for intended analysis.

9b. Age the slides by placing slides in a coplin jar containing 2×SSC at 37° C. for 30 minutes. Pass slide through an ethanol series, 70%, 85% and 100% for one minute each. Allow to air dry. Alternatively, allow the slides to age at Room Temperature in a slide box for 1 to 4 weeks. (See Note 5 below)

10. Store slides at −20° C. in dry containers for long-term storage.

11. Storage of remaining specimen. When an adequate number of slides have been made, store the 15 mL conical tube containing the remaining cell suspension in fixative at −20° C.

### Notes

View the slide preparation with phase contrast microscopy to assess the cell density and metaphase spreading.

1. If the cell density is too high (more than approximately 100 nuclei per 10× field on the phase contrast microscope), add several drops of fixative to the cell suspension in the 15 mL conical and repeat steps for dropping cells on a new slide.

2. If the cell density is too sparse (less than ten nuclei per 10× field), centrifuge the 15 mL conical centrifuge tube containing the cells at 200×g for 5 minutes, aspirate the excess fixative, resuspend the pellet in less fixative than added initially, and repeat steps for dropping cells with a new slide.

3. If there is inadequate spreading so that the majority of chromosomes are indistinguishable, decrease airflow, increase humidity, or decrease temperature to allow the slide to dry slower. If there is over-spreading so that cell boundaries are not distinguishable, increase airflow, decrease humidity, or ambient increase temperature to allow slides to dry faster.

4. The resulting metaphase cells should have minimal overlaps and no visible cytoplasm, with chromosomes appearing as medium gray to dark gray under phase contrast microscopy.

5. Aging of cytogenetic preparations denatures the proteins, removes residual water and fixative, and enhances the adherence of the material to the glass. When fresh, non-aged slides are heat denatured they either lose most of their material or their chromosomes become distorted and puffy in appearance. If slides are aged extensively, hybridization efficiency decreases because the chromosomes are too hard.

### C. Preparation of Peripheral Blood Cells for Chromosome Analysis

The purpose of this protocol is to culture and harvest human lymphocytes to determine structural and numerical chromosomal abnormalities and to prepare chromosome preparations for FISH/ISH hybridization procedures. Specimen

Collect 3-5 mL of heparinized whole blood (green top vacutainer tube); sodium heparin is the recommended anticoagulant.

### Reagents and Instrumentation

| Item | Supplier | Catalog No. |
|---|---|---|
| Acetic acid, glacial, 500 mL | VWR | JT9511-5 |
| Methanol, 1.0 L | VWR | JT9049-2 |
| KaryoMAX Colcemid Solution (10 μg/mL), | Gibco | 15212012 |
| 10 mL | BRL | |
| KaryoMAX ® Potassium Chloride Solution, | Gibco | 10575090 |
| 0.075M | BRL | |
| PB-MAX ™ Karyotyping Medium (1X), liquid | Gibco | 12557021 |
| | BRL | |
| Portable Pipet-Aid device, rechargeable | VWR | 3498-103 |
| T25 culture flask with vent cap, non-treated, | VWR | 89092-698 |
| Corning | | |
| Benchtop Centrifuge, 4x 100 mL capacity | VWR | 53513-800 |
| BD Falcon Centrifuge Tubes, conical, 15 mL | VWR | |
| Serological Pipettes, Disposable, Plugged, 1 mL, | VWR | |
| 2 mL, 5 mL, 10 mL, 25 mL | | |
| VWR Superfrost Plus Micro slide, pack of 72 | VWR | 48311-703 |
| Water bath, 37° C. | | |
| Incubator with 5% CO_{2,} 37° C. | | |

### Preparation

PB-MAX Karyotyping Medium (1×): Thaw PB-MAX Karyotyping medium at 4° C. to 8° C. Warm the medium to room temperature and gently swirl to mix prior to use. PB-MAX Karyotyping medium can be thawed and aseptically transferred into smaller aliquots for convenience. These aliquots can be frozen and thawed at time of use, however multiple freeze-thaw cycles should be avoided. Avoid prolonged exposure to light when using this culture medium product.

Fixative: Methanol:glacial acetic acid, 3:1. Prepare before each use.

KaryoMAX Potassium Chloride Solution, 0.075 M: Prewarm the hypotonic solution to 37° C. prior to use.

### Procedure

Prepare mitotic cells from short-term blood cultures.

1. Add 10 mL of PB-MAX Karyotyping Medium to each sterile T-25 flask to be set up for the assay. (See Note 1)

2. Add 0.75 mL of heparinized blood to each T-25 flask.

3. Incubate for 72 hr at 37° C. (5% CO₂) in a cell culture incubator. Flasks should stand upright with caps loosely closed.

4. After 72 hr culture add 100 μl KaryoMAX Colcemid Solution (10 ug/mL) to each flask and mix well. Incubate for 30 min at 37° C.

5. After 30 minutes, transfer the culture to 15 mL centrifuge tubes and centrifuge at 1200 rpm for 10 min. Remove medium completely except for about 0.5 mL of supernatant remaining above the cell pellet.

6. Resuspend the cells gently in the remaining medium and carefully add approximately 2 mL of prewarmed (37° C.) KaryoMAX Potassium Chloride Solution, 0.075 M, drop-by-drop, while agitating gently. Add an additional 8 mL of KCl, for a total of 10 mL; mix well. (See Note 2)

7. Incubate for 15 min at 37° C. in the water bath.

8. Add 0.5 mL of freshly prepared fixative, recap the tube, and invert to mix.

9. Centrifuge the cells at 1200 rpm for 5 minutes, and remove the supernatant as in step 5.

10. Resuspend the cells and fix the cells by adding 10 mL of fixative; the first 2 mL should be added drop wise while agitating gently.

11. Incubate at for 10 minutes at room temperature, centrifuge the cells and remove the supernatant as in step 5.

12. Repeat the fixation procedure two more times. It is not necessary to incubate the cells between centrifugations.

13. After the last centrifugation, resuspend the cells in 5.0 mL of fixative.

14. Store cell pellets in fixative at −20° C.

### Notes

1. White blood cells in peripheral blood must be stimulated with a mitogen, inducing cell division as a prerequisite for preparation of cells in metaphase. In preparations of peripheral human blood cells, T-lymphocytes are stimulated with phytohemagglutinin. PB-MAX Karyotyping Medium is composed of a liquid RPMI-1640 medium that is completely supplemented with standard concentrations of L-glutamine, gentamicin sulfate, fetal bovine serum and phytohemagglutinin. This formulation is based on Peripheral Blood Media referenced in ACT Laboratory manual (1991) for use in PHA-stimulated Peripheral Blood Culture.

2. Hypotonic treatment causes a swelling of the cells; the optimal time of treatment varies for different cell types and must be determined empirically.

### D. CEP (Chromosome Enumeration Probe) FISH Protocol

Labeled CEP (Chromosome Enumeration Probes) DNA probes can be used to identify human chromosomes in metaphase spreads and interphase nuclei with fluorescence in situ hybridization (FISH) for example to identify aneuploidies in normal and tumor cells, to serve as reference probe in cytogenetic studies and to identify the human chromosomes in hybrid cell lines.

### Specimen

Metaphase chromosomes and/or interphase nuclei of fixed cultured or uncultured cytological specimens prepared on microscope slides.

### Reagents and Instrumentation

| Item | Supplier | Catalog No. |
|---|---|---|
| Rainin Classic Starter Kit. 20/200/1000 μl | Rainin | PR-Start |
| Pipettes | | |
| Rainin PR-10, 0.5-10 uL | Rainin | PR-10 |
| Removable-cover racked tips 10 μl. Presterilized | Rainin | RT-10S |
| Removable-cover racked tips 20 μl. Presterilized | Rainin | RT-20S |
| Removable-cover racked tips 200 μl. Presterilized | Rainin | RT-200S |
| Removable-cover racked tips 1000 μl. | Rainin | RT-1000S |
| Presterilized | | |
| Slide Warmer Space Saver, 120 V | VWR | 15160-795 |
| Analog Water Bath, 2.0 L 37° C. | VWR | 89032-196 |
| Analog Water Bath, 2.0 L 70° C. | VWR | 89032-196 |
| Microcentrigfuge Tubes (1.5 mL), natural, | VWR | 20170-650 |
| qty 250 | | |
| MiniFuge, 200 g, 6000 rpm, 120 V | VWR | 93000-196 |
| VWR Traceable Multi-colored Timer | VWR | 89087-400 |
| 60 mL (2.0 oz) glass coplin jar, case 6 | VWR | 25457-006 |
| Coplin Staining Jar, SCIENCEWARE, each | VWR | 47751-792 |
| VWR Cover Glass Forceps, straight | VWR | 82027-396 |
| VWR Slide Hybridization Oven, or 42° C. | VWR | 80087-000 |
| Incubator | | |
| Rubber Cement | VWR | 100491-938 |
| VWR Clear Bath, algicide, 8 0z. | VWR | 54847-540 |
| 20 x SSC, 1.0 L, DEPC treated | VWR | RLMB-045 |
| Ethanol Series 70%, 85%, 100% | | |
| Formamide, 500 mL | VWR | JTM520-7 |
| Kimwipes | | |
| CEP 4 SpectrumOrange Probe | Abbott | 06J36-014 |
| CEP 17 (D17Z1) SpectrumGreen Probe | Abbott | 06J37-027 |
| CEP Hybridization Buffer, 2 × 150 μL | Abbott | 07J36-001 |
| DAPI II Counterstain, 500 μL × 2 | Abbott | 06J50-001 |
| Antifade Solution, 240 μL × 2 | Abbott | 06J29-010 |
| Control low-level - female, 95% XY, 5% XX | Abbott | 07J21-011 |
| Epifluoresence Microscope with filters and | | |
| Imaging System | | |

### Preparation

Note: Where indicated, measure the pH of these solutions at ambient temperature. Use a pH meter with a glass electrode unless otherwise noted.

2×SSC solution: Mix thoroughly 100 mL 20×SSC (pH 5.3) with 850 mL purified H₂O. Measure pH and adjust to pH 7.0±0.2 with NaOH. Add purified H₂O to bring final volume to 1 liter. Store at ambient temperature. Discard stock solution after 6 months, or sooner if solution appears cloudy or contaminated. Prepare 2×SSC in plastic coplin jar and preheat to 37° C. using a water bath.

Denaturation Solution (70% Formamide/2×SSC): Mix thoroughly 49 mL ultrapure formamide, 7 mL 20×SSC (pH 5.3) and 14 mL purified H₂O in a glass coplin jar. Measure pH using pH indicator strips to verify pH is 7.0-8.0. Between uses, store covered at 2-8° C. Discard after 7days. Prepare in glass coplin jar and heat to 73+/−1° C.

0.4×SSC/0.3% NP-40 Wash Solution: Mix thoroughly 20 mL 20×SSC (pH 5.3) with 950 mL purified H₂O. Add 3 mL of NP-40. Mix thoroughly until NP-40 is completely dissolved. Measure pH and adjust pH to 7.0-7.5 with NaOH. Add purified H₂O to bring final volume of the solution to 1 liter. Store at ambient temperature. Discard stock solution after 6 months, or sooner if solution appears cloudy or contaminated. Prepare in glass coplin jar and heat to 73+/−1° C.

2×SSC/0.1% NP-40 Wash Solution: Mix thoroughly 100 mL 20×SSC (pH 5.3) with 850 mL purified H₂O. Add 1 mL NP-40. Measure pH and adjust to pH 7.0±0.2 with NaOH. Add purified H₂O to bring final volume to 1 liter. Store at ambient temperature. Discard stock solution after 6 months, or sooner if solution appears cloudy or contaminated. Prepare in glass coplin jar and heat to 73+/−1° C.

Ethanol Solutions (70%, 85%, 100%): Prepare v/v dilutions of 100% ethanol with purified H₂O. Between uses, store tightly covered at ambient temperature. Discard stock solutions after 6 months. Prepare 70%, 85% and 100% ethanol using distilled water in plastic coplin jars.

Fluorescence in situ Hybridization Procedure

### Probe Preparation

At room temperature mix 7 μL of CEP hybridization buffer, 1 μL CEP DNA probe, and 2 μL purified H₂O. Centrifuge for 1-3 seconds, vortex and then re-centrifuge.

Heat for 5 minutes in a 73° C. water bath, and then place on a slide warmer set to 45-50° C.

Vortex to mix. Spin the tubes briefly (1-3 seconds) in microcentrifuge to bring the contents to the bottom of the tube. Gently vortex again to mix.

### Denaturation of Specimen DNA (Control Slides or PHA-Stimulated Peripheral Blood Lymphocytes)

Prewarm the hybridization chamber (an airtight container) to 42° C. by placing it in the 42° C. incubator prior to slide preparation.

Add denaturing solution to Coplin jar and place in a 73±1° C. water bath for at least 30 minutes. Verify the solution temperature before use.

Denature the specimen DNA by immersing the prepared slides in the denaturing solution at 73±1° C. for 5 minutes. Do not denature more than 4 slides at one time per Coplin jar. Check that the pH of the denaturing solution is 7.0-8.0 before each use.

Using forceps remove the slide(s) from the denaturing solution and immediately place into a 70% ethanol wash solution at room temperature. Agitate the slide to remove the formamide. Allow the slide(s) to stand in the ethanol wash for 1 minute.

Remove the slide(s) from 70% ethanol. Repeat step 4 with 85% ethanol, followed by 100% ethanol.

Drain the excess ethanol from the slide by touching the bottom edge of the slide to a blotter and wipe the underside of the slide dry with a laboratory wipe.

Place the slide(s) on a 45-50° C. slide warmer no more than 2 minutes before you are ready to apply the probe solution.

Note: If the timing of the hybridization is such that the slide is ready more than 2 minutes before the probe is ready, the slide should remain in the jar of 100% ethanol. Do not air dry a slide before placing it on the slide warmer.

### Hybridization

Apply the 10 μL aliquot of probe solution to the target area of the slide. Immediately, place a 22 mm×22 mm glass coverslip over the probe solution and allow the solution to spread evenly under the coverslip. Air bubbles will interfere with hybridization and should be avoided.

Note: Do not pipet probe solution onto multiple target areas before applying the coverslips.

Place the slide into the pre-warmed 42° C. hybridization chamber and cover the chamber with a tight lid.

Place the chamber containing the slide into the 42° C. incubator and allow hybridization to proceed for at least 30 minutes.

Note: Longer hybridization time may be required for sufficient signal intensity in some specimens. Incubations may be performed overnight (16 hours). For incubations longer than 1 hour, the coverslip must be sealed using a removable sealant such as rubber cement and the hybridization chamber must be humidified. The procedure is described below.

Draw rubber cement into a 5 mL syringe. Exude a small amount of rubber cement around the periphery of the coverslip overlapping the coverslip and the slide, thereby forming a seal around the coverslip.

Place the slide into a humidified hybridization chamber (an airtight container with a piece of damp blotting paper or paper towel approximately 1 in.×3 in. taped to the side of the container).

Cover the chamber with a tight lid and incubate 1 to 16 hours, as desired.

Following incubation, remove the rubber cement from the coverslip by pulling up on the rubber cement.

### Post-Hybridization Washes

Add 0.4×SSC (pH 7.0-7.5) to a Coplin jar. Prewarm the 0.4×SSC solution by placing the Coplin jar in the 73±1° C. water bath for at least 30 minutes or until the solution temperature has reached 73±1° C.

Note: In order to maintain the proper temperature range, four slides should be placed in the heated wash solution at one time. If fewer than four slides have been hybridized, room temperature microscope slides (without specimen applied) may be used to bring the number of slides to four. If more than four slides have been hybridized they must be washed in more than one batch. The temperature of the wash solution must return to 73±1° C. before washing each batch.

Remove the coverslip from the target area of the first slide and immediately place the slide into the Coplin jar containing 0.4×SSC, 73±1° C. Agitate the slide for 1-3 seconds. Repeat for the other three slides and incubate for 2 minutes at 73±1° C.

Note: Do not remove the coverslips from several slides before placing any of the slides in the wash bath. Begin timing the 2 minute incubation when the last slide has been added to the wash bath.

Remove each slide from the wash bath and place in the jar of 2×SSC/0.1% NP-40 at room temperature for 5-60 seconds, agitating for 1-3 seconds as the slides are placed in the bath.

Allow the slide to air dry in the dark. (A closed drawer or a shelf inside a closed cabinet is sufficient.)

Apply 10 μL of DAPI II counterstain to the target area of the slide and apply a glass coverslip. Store the slide(s) in the dark prior to signal enumeration.

### Storage

Store hybridized slides (with coverslips) at −20° C. in the dark. Under these conditions the slides can be stored for up to 12 months without significant loss in fluorescence signal intensity. For long-term storage, the coverslips should be sealed to prevent desiccation and the slides stored at −20° C.

### Signal Enumeration-Assessing Slide Adequacy

Evaluate slide adequacy using the following criteria:

Probe Signal Intensity: The signal should be bright, distinct, and easily evaluable. Signals should be in either bright, compact, oval shapes or stringy, diffuse, oval shapes.

Background: The background should appear dark or black and free of fluorescence particles or haziness.

Cross-hybridization/Target Specificity: The probe should hybridize and illuminate only the target (centromere of chromosome). Metaphase spreads should be evaluated to verify locus specificity and to identify any cross-hybridization to non-target sequences. At least 98% of cells should show one or more signals for acceptable hybridization. Signal Enumeration-Selection of optimum viewing area and evaluable nuclei

Use a 25× objective to scan the hybridized area and examine the specimen distribution. Select an area where the specimen is distributed sparsely, few interphase nuclei are overlapping, and several interphase nuclei can be scanned within a viewing field. Avoid areas where the distribution of cells is dense, cells are overlapped, or the nuclear border of individual nuclei is unidentifiable. Avoid areas that contain clumps of cells. Enumerate only those cells with discrete signals.

### Signal Enumeration-Enumeration Scan

Using a 40× or 63× objective, begin analysis in the upper left quadrant of the selected area and, scanning from left to right, count the number of signals within the nuclear boundary of each evaluable interphase cell. Areas on the slide with a high cell density should be randomly skipped in order to scan the entire target area. Continue the scanning until 500 interphase nuclei are enumerated and analyzed.

### Signal Enumeration-Interphase Enumeration

Enumerate the fluorescent signals in each evaluable interphase nucleus using a 40× or 63× objective. Objectives with higher magnification (e.g., 63× or 100× ) should be used to verify or resolve questions about split or diffused signals. Follow these guidelines:

Two signals that are in close proximity and approximately the same sizes but not connected by a visible link are counted as two signals.

Count a diffuse signal as one signal if diffusion of the signal is contiguous and within an acceptable boundary.

Two small signals connected by a visible link are counted as one signal.

Enumerate the number of nuclei with 0, 1, 2, 3, 4, or >4 signals. Count nuclei with zero signals only if there are other nuclei with at least one signal present in the field of view. If the accuracy of enumeration is in doubt, repeat the enumeration in another area of the slide.

Do not enumerate nuclei with uncertain signals.

### E. LSI (Locus Specific Identifier) FISH Protocol

The purpose of this protocol is to perform FISH using LSI (Locus Specific Identifier) probes on cytogenetic specimens. Labeled LSI DNA probes can be used to identify human chromosomes in metaphase spreads and interphase nuclei, and genetic aberrations with fluorescence in situ hybridization (FISH). For example the LSI BCR/ABL probe set is designed to detect fusion of the ABL gene locus on 9q34 and BCR gene locus on 22q11.2 (Translocation (9;22)(q34;q11)).

### Specimen

Metaphase chromosomes and/or interphase nuclei of fixed cultured or uncultured cytological specimens prepared on microscope slides.

### Reagents and Instrumentation

| Item | Supplier | Catalog No. |
|---|---|---|
| Rainin Classic Starter Kit. 20/200/1000 μl | Rainin | PR-Start |
| Pipettes | | |
| Rainin PR-10, 0.5-10 uL | Rainin | PR-10 |
| Removable-cover racked tips 10 μl. Presterilized | Rainin | RT-10S |
| Removable-cover racked tips 20 μl. Presterilized | Rainin | RT-20S |
| Removable-cover racked tips 200 μl. Presterilized | Rainin | RT-200S |
| Removable-cover racked tips 1000 μl. | Rainin | RT-1000S |
| Presterilized | | |
| Slide Warmer Space Saver, 120 V | VWR | 15160-795 |
| Analog Water Bath, 2.0 L 37° C. | VWR | 89032-196 |
| Analog Water Bath, 2.0 L 70° C. | VWR | 89032-196 |
| Microcentrigfuge Tubes (1.5 mL), natural, qty | VWR | 20170-650 |
| 250 | | |
| MiniFuge, 200 g, 6000 rpm, 120 V | VWR | 93000-196 |
| VWR Traceable Multi-colored Timer | VWR | 89087-400 |
| 60 mL (2.0 oz) glass coplin jar, case 6 | VWR | 25457-006 |
| Coplin Staining Jar, SCIENCEWARE, each | VWR | 47751-792 |
| VWR Cover Glass Forceps, straight | VWR | 82027-396 |
| VWR Slide Hybridization Oven, or 42° C. | VWR | 80087-000 |
| Incubator | | |
| Rubber Cement | VWR | 100491-938 |
| VWR Clear Bath, algicide, 8 0z. | VWR | 54847-540 |
| 20 x SSC, 1.0 L, DEPC treated | VWR | RLMB-045 |
| Ethanol Series 70%, 85%, 100% | | |
| Formamide, 500 mL | VWR | JTM520-7 |
| Kimwipes | | |
| Vysis LSI BCR/ABL Dual Color, Single Fusion | Abbott | 05J77-001 |
| Translocation Probe | | |
| LSI Hybridization Buffer, 2 × 150 μL | Abbott | 07J36-001 |
| DAPI II Counterstain, 500 μL × 2 | Abbott | 06J50-001 |
| Antifade Solution, 240 μL × 2 | Abbott | 06J29-010 |
| Control low-level—female, 95% XY, 5% XX | Abbott | 07J21-011 |
| Epifluoresence Microscope with filters and | | |
| Imaging System | | |

### Preparation

Where indicated, measure the pH of these solutions at ambient temperature. Use a pH meter with a glass electrode unless otherwise noted.

2×SSC solution: Mix thoroughly 100 mL 20×SSC (pH 5.3) with 850 mL purified H₂O. Measure pH and adjust to pH 7.0±0.2 with NaOH. Add purified H₂O to bring final volume to 1 liter. Store at ambient temperature. Discard stock solution after 6 months, or sooner if solution appears cloudy or contaminated. Prepare 2×SSC in plastic coplin jar and preheat to 37° C. using a water bath.

Denaturation Solution (70% Formamide/2×SSC):Mix thoroughly 49 mL ultrapure formamide, 7 mL 20×SSC (pH 5.3) and 14 mL purified H₂O in a glass coplin jar. Measure pH using pH indicator strips to verify pH is 7.0-8.0. Between uses, store covered at 2-8° C. Discard after 7 days. Prepare in glass coplin jar and heat to 73+/−1° C.

0.4×SSC/0.3% NP-40 Wash Solution: Mix thoroughly 20 mL 20×SSC (pH 5.3) with 950 mL purified H₂O. Add 3 mL of NP-40. Mix thoroughly until NP-40 is completely dissolved. Measure pH and adjust pH to 7.0-7.5 with NaOH. Add purified H₂O to bring final volume of the solution to 1 liter. Store at ambient temperature. Discard stock solution after 6 months, or sooner if solution appears cloudy or contaminated. Prepare in glass coplin jar and heat to 73+/−1° C.

2×SSC/0.1% NP-40 Wash Solution: Mix thoroughly 100 mL 20×SSC (pH 5.3) with 850 mL purified H₂O. Add 1 mL NP-40. Measure pH and adjust to pH 7.0±0.2 with NaOH. Add purified H₂O to bring final volume to 1 liter. Store at ambient temperature. Discard stock solution after 6 months, or sooner if solution appears cloudy or contaminated. Prepare in glass coplin jar and heat to 73+/−1° C.

Ethanol Solutions (70%, 85%, 100%): Prepare v/v dilutions of 100% ethanol with purified H₂O. Between uses, store tightly covered at ambient temperature. Discard stock solutions after 6 months. Prepare 70%, 85% and 100% ethanol using distilled water in plastic coplin jars.

### LSI Probe Preparation

At room temperature mix 7 ul of LSI Hybridization Buffer, 1 ul LSI DNA probe, and 2 ul purified H₂O. Centrifuge for 1-3 seconds, vortex and then re-centrifuge. Place on ice until use.

Fluorescence In Situ Hybridization Procedure

Denaturation of Specimen DNA (Control Slides or PHA-Stimulated Peripheral Blood Lymphocytes)

Prewarm the hybridization chamber (an airtight container) to 37° C. by placing it in the 37° C. incubator prior to slide preparation.

Add denaturing solution to Coplin jar and place in a 73±1° C. water bath for at least 30 minutes. Verify the solution temperature before use.

Denature the specimen DNA by immersing the prepared slides in the denaturing solution at 73±1° C. for 5 minutes. Do not denature more than 4 slides at one time per Coplin jar. Check that the pH of the denaturing solution is 7.0-8.0 before each use.

Using forceps remove the slide(s) from the denaturing solution and immediately place into a 70% ethanol wash solution at room temperature. Agitate the slide to remove the formamide. Allow the slide(s) to stand in the ethanol wash for 1 minute.

Remove the slide(s) from 70% ethanol. Repeat step 4 with 85% ethanol, followed by 100% ethanol.

Drain the excess ethanol from the slide by touching the bottom edge of the slide to a blotter and wipe the underside of the slide dry with a laboratory wipe.

Place the slide(s) on a 45-50° C. slide warmer no more than 2 minutes before you are ready to apply the probe solution.

Note: If the timing of the hybridization is such that the slide is ready more than 2 minutes before the probe is ready, the slide should remain in the jar of 100% ethanol. Do not air dry a slide before placing it on the slide warmer.

### Probe Preparation

Heat the prepared probe for 5 minutes in a 73° C. water bath.

Place on a slide warmer set to 45-50° C. Cover tube with foil to block form light if not using right away.

### Hybridization

Apply the 10 μL aliquot of probe solution to the target area of the slide. Immediately, place a 22 mm×22 mm glass coverslip over the probe solution and allow the solution to spread evenly under the coverslip. Air bubbles will interfere with hybridization and should be avoided. Seal the coverslip with rubber cement.

Note: Do not pipet probe solution onto multiple target areas before applying the coverslips.

Place the slide into the pre-warmed 37° C. hybridization chamber and cover the chamber with a tight lid.

Place the chamber containing the slide into the 37° C. incubator and allow hybridization to proceed for 12-16 hours.

Post-Hybridization Washes

Add 0.4×SSC (pH 7.0-7.5) to a Coplin jar. Prewarm the 0.4×SSC solution by placing the Coplin jar in the 73±1° C. water bath for at least 30 minutes or until the solution temperature has reached 73±1° C.

Note: In order to maintain the proper temperature range, four slides MUST be placed in the heated wash solution at one time. If fewer than four slides have been hybridized, room temperature microscope slides (without specimen applied) may be used to bring the number of slides to four. If more than four slides have been hybridized they must be washed in more than one batch. The temperature of the wash solution must return to 73±1° C. before washing each batch.

Remove the rubber cement and coverslip from the target area of the first slide and immediately place the slide into the Coplin jar containing 0.4×SSC, 73±1° C. Agitate the slide for 1-3 seconds. Repeat for the other three slides and incubate for 2 minutes at 73±1° C.

Note: Do not remove the coverslips from several slides before placing any of the slides in the wash bath. Begin timing the 2 minute incubation when the last slide has been added to the wash bath.

Remove each slide from the wash bath and place in the jar of 2×SSC/0.1% NP-40 at room temperature for 5-60 seconds, agitating for 1-3 seconds as the slides are placed in the bath.

Allow the slide to air dry in the dark. (A closed drawer or a shelf inside a closed cabinet is sufficient.)

Apply 10 μL of DAPI II counterstain to the target area of the slide and apply a glass coverslip. Store the slide(s) in the dark prior to signal analysis.

### Storage

Store hybridized slides (with coverslips) at −20° C. in the dark. Under these conditions the slides can be stored for up to 12 months without significant loss in fluorescence signal intensity. For long-term storage, the coverslips should be sealed to prevent desiccation and the slides stored at −20° C.

### Signal Analysis

### Assessing Slide Adequacy

The Triple bandpass filter DAPI/FITC/Texas Red is optimal for viewing all three fluorophores simultaneously. Evaluate slide adequacy using the following criteria:

Probe Signal Intensity: The signal should be bright, distinct, and easily evaluable. Signals should be in either bright, compact, oval shapes or stringy, diffuse, oval shapes.

Background: The background should appear dark or black and free of fluorescence particles or haziness.

Cross-hybridization/Target Specificity: The probe should hybridize and illuminate only the target. Metaphase spreads should be evaluated to verify locus specificity and to identify any cross-hybridization to non-target sequences.

### Selection of Optimum Viewing Area and Evaluable Nuclei

Use a 25× objective to scan the hybridized area and examine the specimen distribution. Select an area where the specimen is distributed sparsely, few interphase nuclei are overlapping, and several interphase nuclei can be scanned within a viewing field. Avoid areas where the distribution of cells is dense, cells are overlapped, or the nuclear border of individual nuclei is unidentifiable. Avoid areas that contain clumps of cells. Analyze only those cells with discrete signals.

### Interphase Enumeration

Analyze the fluorescent signals in each evaluable interphase nucleus using a 63× or 100× objective. In a normal cell, these probes will appear as discrete red (R) and green (G) spots, one for each homologue (resulting in a 2G 2R conformation). In a t(9:22) patient, there should be one yellow, white, or yellow-white (Y) fusion signal in addition to the red and green signals of the normal chromosome 9 and 22 respectively (1R 1G 1Y).

### Example 9

### Relationship Between Gleason Scores and Gene Expression

In a study of prostate tissue obtained from 12 male patients, the relationships among gene expression (at varying sites) and certain prognostic variables were investigated.

The gene expression variables measured included the levels of FAS (Fatty Acid Synthase; GenBank NM_{—}004104; SEQ ID NO: 6), USP2a, (ubiquitin specific peptidase 2; GenBank NM 004205 (isoform Hong variant; SEQ ID NO. 7), pAKT (v-akt murine thymoma viral oncogene homolog 1; GenBank NM_{—}005163 variant 1: long version; SEQ ID NO. 8) and NPY (Neuropeptide Y; GenBank NM_{—}000905; SEQ ID NO. 9) on a grade scale of 0, trace, 1, 2 or 3 where 0 represents no expression detected. It should be noted that zero or no expression was set equivalent to baseline expression of the gene in normal tissue. Hence a grade or level of 0 means expression was equivalent to normal tissue expression of the gene in question. Trace means that more than 0 but less than 1% over normal tissue expression was detected, 1 means that 1-25% expression over normal tissue was detected, 2 means that 25-75% expression over normal tissue was detected, and 3 means that 75-100% expression over normal tissue was detected as compared to control. In normal tissue, there is always a trace amount of FAS, USP2a and NPY. In at least 90% of cases, pAKT is found in normal tissue. How the expression levels are related to Gleason Scores (alone or in combination) was also evaluated.

In addition, the study investigated (a) whether there is a significant difference in gene expression between the cancerous sites (Cancer) and a site from the margin of a cancerous site (Margin) and (b) whether there is a significant difference in gene expression between sites from the margin of a cancerous site (Margin) and noncancerous sites (Normal). The data are shown in Table 5.

TABLE 5
Comparative Analysis of Gene Expression

| | | | | | | | | |
|---|---|---|---|---|---|---|---|---|
| Pt. | Age | T | Surgery | Dx | FAS | USP2a | pAkt | NPY |

Patients with four measurements
(3 from cancerous site and one from non-cancerous site)

| | | | | | | | | |
|---|---|---|---|---|---|---|---|---|
| A | 85 | 0 | Biopsy | Adeno CA | 1 | 3 | 1 | C3 |
| | | | | GL6 (3 + 3) | | | | |
| A | 85 | 0 | Biopsy | Adeno CA | trace | 3 | 2 | C1 |
| | | | | GL6 (3 + 3) | | | | |
| A | 85 | 0 | Biopsy | Adeno CA | 0 | 3 | 2 | C3 |
| | | | | GL6 (3 + 3) | | | | |
| A | 85 | | | Normal | none | none | none | C2 |
| B | 84 | T1b | Resection | Adeno CA | 0 | 2 | 1 | C2 |
| | | | | GL6 (3 + 3) | | | | |
| B | 84 | T1b | Resection | Adeno CA | none | none | none | NC |
| | | | | GL6 (3 + 3) | | | | |
| B | 84 | T1b | Resection | Adeno CA | 1 | 3 | 1 | C1 |
| | | | | GL6 (3 + 3) | | | | |
| B | 84 | | | Normal | 1 | 3 | 2 | C1; N trace |
| C | 79 | 1 | Resection | Adeno CA | 3 | 2 | 2 | C1, N trace |
| | | | | GL9 (4 + 5) | | | | |
| C | 79 | 1 | Resection | Adeno CA | 3 | 2 | 2 | C1 |
| | | | | GL9 (4 + 5) | | | | |
| C | 79 | 1 | Resection | Adeno CA | 3 | 2 | 2 | C1, N trace |
| | | | | GL9 (4 + 5) | | | | |
| C | 79 | | | Normal | 3 | 2 | 1 | C1 |
| | | | | | (tumor) | (tumor) | (tumor) | (tumor) |
| D | 74 | 1 | Resection | Adeno CA | 3 | 3 | 1 | C1, N1 |
| | | | | GL8 (4 + 4) | | | | |
| D | 74 | 1 | Resection | Adeno CA | none | none | none | none |
| | | | | GL8 (4 + 4) | | | | |
| D | 74 | 1 | Resection | Adeno CA | 3 | 3 | 1 | C1, N1 |
| | | | | GL8 (4 + 4) | | | | |
| D | 74 | | | Normal | 2 | 3 | 1 | C1, N1 |
| E | 68 | T1b | Resection | Benign + | 2 | 2 | 0 | C1, N1 |
| | | | | Cancer | | | | |
| | | | | GL3 | | | | |
| E | 68 | T1b | Resection | Benign + | 2 | 2 | 1 | C1, N trace |
| | | | | Cancer | | | | |
| | | | | GL3 | | | | |
| E | 68 | T1b | Resection | Benign + | 2 | 2 | 1 | C1, N1 |
| | | | | Cancer | | | | |
| | | | | GL3 | | | | |
| E | 68 | | | Normal | 1 | 3 | 2 | C2 |
| F | 78 | T1b | Resection | Adeno CA | 2 | 1 | 1 | C1 |
| | | | | GL7 (4 + 3) | | | | |
| F | 78 | T1b | Resection | Adeno CA | 2 | 2 | 1 | C1 |
| | | | | GL7 (4 + 3) | | | | |
| F | 78 | T1b | Resection | Adeno CA | NG | NG | NG | NG |
| | | | | GL7 (4 + 3) | | | | |
| F | 78 | | | Normal | NG | NG | NG | NG |
| G | 78 | T1b | Resection | Adeno CA | 3 | 3 | 3 | C2 |
| | | | | GL8-9 | | | | |
| G | 78 | T1b | Resection | Adeno CA | 3 | 3 | 3 | C2, N1 |
| | | | | GL8-9 | | | | |
| G | 78 | T1b | Resection | Adeno CA | 3 | 3 | 3 | C2, N trace |
| | | | | GL8-9 | | | | |
| G | 78 | | | Normal | NG | NG | NG | NG |
| H | 65 | T1b | Resection | Carcinoma | 3 | 1 | 2 | C2, N1 |
| | | | | GL8 (4 + 4) | | | | |
| H | 65 | T1b | Resection | Carcinoma | 3 | 1 | 1 | NG |
| | | | | GL8 (4 + 4) | | | | |
| H | 65 | T1b | Resection | Carcinoma | 2 | 2 | 1 | C3, N2 |
| | | | | GL8 (4 + 4) | | | | |
| H | 65 | | | Normal | 1 | 3 | 2 | C3 |
| I | 65 | 1 | Resection | Mixed | NG | NG | NG | NG |
| | | | | Adeno CA | | | | |
| I | 65 | 1 | Resection | Mixed | 2 | 3 | 2 | C1, N1 |
| | | | | Adeno CA | | | | |
| I | 65 | 1 | Resection | Mixed | 2 | 3 | 3 | C1, N1 |
| | | | | Adeno CA | | | | |
| I | 65 | | | Normal | NG | NG | NG | NG |
| J | 80 | 1 | Resection | Adeno CA | 2 | 2 | 1 | C2, N1 |
| | | | | GL9 | | | | |
| | | | | (5 + 4)) | | | | |
| J | 80 | 1 | Resection | Adeno CA | NG | NG | NG | NG |
| | | | | GL9 (5 + 4) | | | | |
| J | 80 | 1 | Resection | Adeno CA | NG | cautery; | cautery; | NG |
| | | | | GL9 (5 + 4) | | no | no | |
| | | | | | | intact | intact | |
| | | | | | | glands | glands | |
| J | 80 | | | Normal | NG | NG | NG | C2, N trace |
| K | 75 | T1b | Resection | NULL | T: 3 | T: 3 | T: 2 | T: C1, N1 |
| K | 75 | T1b | Resection | NULL | T: 3 | T: 3 | T: 1 | T: C1, N1 |
| K | 75 | T1b | Resection | NULL | T: 3 | T: 3 | T: 1 | T: C1, N1 |
| K | 75 | | | Normal | 2 | 3 | 2 | C2, N trace |
| L | 94 | TIS | Resection | Focal | N: 2 | N: 3 | N: 2 | normal; C1, |
| | | | | Adeno CA | | | | N trace |
| | | | | GL6 (3 + 3) | | | | |
| L | 94 | TIS | Resection | Focal | N: 2 | N: 3 | N: 2 | normal: C1, |
| | | | | Adeno CA | | | | N trace; |
| | | | | GL6 (3 + 3) | | | | tumor: C1, N |
| | | | | | | | | trace |
| L | 94 | TIS | Resection | Focal | 3 | 3 | 2 | C1, N1 |
| | | | | Adeno CA | | | | |
| | | | | GL6 (3 + 3) | | | | |
| L | 94 | | | Normal | NG | NG | NG | distorted |
| | | | | | | | | gland at |
| | | | | | | | | edge; cannot |
| | | | | | | | | evaluate |
| M | 72 | T1b | Resection | Adeno CA | 3 | 2 | 2 | C1, N1 |
| | | | | GL6 (3 + 3) | | | | |
| M | 72 | T1b | Resection | Adeno CA | 3 | 2 | 2 | C1, N1 |
| | | | | GL6 (3 + 3) | | | | |
| M | 72 | T1b | Resection | Adeno CA | 3 | 2 | 2 | C1, N1 |
| | | | | GL6 (3 + 3) | | | | |
| M | 72 | | | Normal | trace | 3 | 1 | C1, N1 |

Patients with only three measurements from cancerous sites

| | | | | | | | | |
|---|---|---|---|---|---|---|---|---|
| N | 64 | 1 | Resection | Adeno CA | 1 | 1 | 2 | C1, N1 |
| | | | | GL9 (5 + 4) | | | | |
| N | 64 | 1 | Resection | Adeno CA | NG | 2 | trace | distorted |
| | | | | GL9 (5 + 4) | | | | glands at |
| | | | | | | | | edge; cannot |
| | | | | | | | | evaluate |
| N | 64 | 1 | Resection | Adeno CA | 2 | 2 | 3 | distorted |
| | | | | GL9 (5 + 4) | | | | glands; |
| | | | | | | | | cannot |
| | | | | | | | | evaluate |
| O | 66 | T1b | NULL | Adeno CA | T: 0 | T: 2 | T: 1 | T: C3 |
| | | | | GL6 (3 + 3) | | | | |
| O | 66 | T1b | NULL | Adeno CA | T: 1 | T: 1 | T: | T: C3, N: 2 |
| | | | | GL6 (3 + 3) | | | trace | |
| O | 66 | T1b | NULL | Adeno CA | none | none | none | none |
| | | | | GL6 (3 + 3) | | | | |
| P | 79 | 1 | Resection | Adeno CA | 2 | 2 | 2 | C1, N trace |
| | | | | GL7 (3 + 4) | | | | |
| P | 79 | 1 | Resection | Adeno CA | 1 | 1 | 2 | C1, N1 |
| | | | | GL7 (3 + 4) | | | | |
| P | 79 | 1 | Resection | Adeno CA | 1 | 1 | 2 | C1, N trace |
| | | | | GL7 (3 + 4) | | | | |
| Q | 71 | 1 | Resection | Adeno CA | 1 | 1 | 1 | C3, N1 |
| | | | | GL9 (5 + 4) | | | | |
| Q | 71 | 1 | Resection | Adeno CA | 1 | 2 | 1 | C3, N2 |
| | | | | GL9 (5 + 4) | | | | |
| Q | 71 | 1 | Resection | Adeno CA | none | none | none | none |
| | | | | GL9 (5 + 4) | | | | |
| R | 64 | 4 | Biopsy | Adeno CA | 2 | 1 | 2 | C trace |
| | | | | GL7 (4 + 3) | | | | |
| R | 64 | 4 | Biopsy | Adeno CA | 1 | 2 | 2 | NG |
| | | | | GL7 (4 + 3) | | | | |
| R | 64 | 4 | Biopsy | Adeno CA | none | none | none | none |
| | | | | GL7 (4 + 3) | | | | |
| S | 69 | X | Biopsy | Carcinoma | 0 | 2 | 2 | C2, N trace |
| | | | | GL6 (3 + 3) | | | | |
| S | 69 | X | Biopsy | Carcinoma | trace | 2 | 3 | C2, N trace |
| | | | | GL6 (3 + 3) | | | | |
| S | 69 | X | Biopsy | Carcinoma | 0 | 2 | 2 | C2, N trace |
| | | | | GL6 (3 + 3) | | | | |
| T | 71 | X | NULL | BPH and | N: 1 | N: 3 | N: 2 | C1, N trace |
| | | | | CA GL6 | | | | |
| | | | | (3 + 3) | | | | |
| T | 71 | X | NULL | BPH and | N: | N: 3 | N: | NG |
| | | | | CA GL6 | trace | | trace | |
| | | | | (3 + 3) | | | | |
| T | 71 | X | NULL | BPH and | N: 1 | N: 3 | N: 2 | normal: C1, |
| | | | | CA GL6 | | | | N trace |
| | | | | (3 + 3) | | | | |
| U | 68 | X | NULL | BPH | N: 1 | N: 3 | N: | normal: C2, |
| | | | | | | | trace | N trace |
| U | 68 | X | NULL | BPH | N: 1 | N: 3 | N, 0 | normal: C2, |
| | | | | | | | N trace | |
| U | 68 | X | NULL | BPH | N: 1 | N: 2 | N: 1 | normal: C1 |
| V | 69 | X | NULL | Hyperplasia & | N: 1 | N: 3 | N: 1 | normal: C1, |
| | | | | Chronic | | | | N trace |
| | | | | Inflammation | | | | |
| V | 69 | X | NULL | Hyperplasia & | N: 1 | v | N: 1 | normal: C1, |
| | | | | Chronic | | | | N trace |
| | | | | Inflammation | | | | |
| V | 69 | X | NULL | Hyperplasia & | N: 1 | N: 2 | N: 2 | normal: C1, |
| | | | | Chronic | | | | N trace |
| | | | | Inflammation | | | | |
| W | 59 | X | Resection | chronic | NG | NG | NG | NG |
| | | | | prostatitis | | | | |
| W | 59 | X | Resection | chronic | NG | NG | NG | NG |
| | | | | prostatitis | | | | |
| W | 59 | X | Resection | chronic | NG | NG | NG | NG |
| | | | | prostatitis | | | | |
| X | 66 | X | NULL | BPH | N: 1 | N: 2 | N: 2 | normal: C1, |
| | | | | | | | | N trace |
| X | 66 | X | NULL | BPH | NG | cautery; | distorted; | NC |
| | | | | | | cannot | cannot | |
| | | | | | | score | score | |
| X | 66 | X | NULL | BPH | N: 1 | N: 2 | N: 1 | normal: C1 |
| Y | 87 | X | NULL | Fibromuscular | none | none | soft | NG |
| | | | | Hyperplasia | | | tissue; | |
| | | | | | | | stain | |
| | | | | | | | present | |
| | | | | | | | in | |
| | | | | | | | ?cells; | |
| | | | | | | | distorted | |
| Y | 87 | X | NULL | Fibromuscular | NG | NG | NG | NG |
| | | | | Hyperplasia | | | | |
| Y | 87 | X | NULL | Fibromuscular | NG | NG | NG | NG |
| | | | | Hyperplasia | | | | |

Table Abbreviations:
T = tumor grade;
N = Nodes;
M = Mets;
Dx = pathological diagnosis;
“Adeno CA” stands for adenocarcinoma;
“GL” stands for Gleason grade or score;
C = cytoplasmic;
N = nuclear (with degrees of staining listed numerically);
NG means No Glands;
NULL as it relates to Surgery means the surgery type was unknown.

### Results of Statistical Analysis: Gene Expression

The data consist of two types of patients. Patients A through M each have four samples. Three samples are from cancerous sites and one from a Normal site. Patients N through Y only contain three samples from cancerous sites. They do not contain samples from Normal sites.

Making a direct comparison of the expression at the Normal site with the three Cancerous sites, the analysis was similar to a “Paired t-Test”, except that the pairs consist of one and three observations. Thus expression at the cancerous site is known with higher precision than at Normal sites.

Using the SAS statistical package, JMP, version 8; (Cary, N.C.), a Fit Model platform was used to adjust for this unequal allocation. In the model, four separate Y-variables interrogated were FAS, USP2a, pAKT and NPY expression. The X-variable was Site (Cancer vs. Margin). Least square means (LSMeans) are averages that are adjusted for different sample sizes and are reported in Table 6.

TABLE 6
Least Square Means for Gene expression by Site
Cancer v. Margin

| | Least Sq | Std | | | | |
|---|---|---|---|---|---|---|
| Site | Mean | Error | Mean | Difference | 95% CI range | p-Value |
| | FAS Expression | | | | | |
| Cancer | 1.836 | 0.149 | 1.836 | | | |
| Margin | 0.675 | 0.258 | 0.675 | | | |
| | | | | 1.161 | 0.557-1.765 | 0.0004 |
| | USP2a Expression | | | | | |
| Cancer | 1.972 | 0.198 | 1.972 | | | |
| Margin | 1.417 | 0.342 | 1.417 | | | |
| | | | | 0.556 | 0.-0.247 to | 0.1687 |
| | | | | | 1.358 | |
| | pAKT Expression | | | | | |
| Cancer | 1.389 | 0.140 | 1.389 | | | |
| Margin | 0.750 | 0.242 | 0.750 | 0.639 | 0.072 to 1.206 | 0.0283 |
| | NPY Expression | | | | | |
| Cancer | 1.139 | 0.132 | 1.139 | | | |
| Margin | 1.169 | 0.242 | 1.182 | 0.043 | — | 0.9130 |

From the table it is evident that Cancerous sites have higher FAS levels than Margin sites. The difference was highly significant. For USP2a, while there was higher expression in the Cancerous sites, the difference was not significant. For pAKT, expression was higher in Cancerous sites and, like FAS, the difference was significant. For NPY there was approximately equal expression in Cancerous and Margin sites.

### Relation to Gleason Score

Based on the limited data set of 48 samples from 12 patients, FAS expression had the strongest relationship with Gleason scores. This relationship was statistically significant with p=0.00687. The other three variables were not significant.

### Evaluation of Expression of Margin and Normal Sites

In an effort to determine whether there is a significant difference between normal sites from the margin of a cancerous tissue and the normal sites from noncancerous tissue, a third dataset from 10 additional patients was generated in which two samples were obtained from either a site near or at the margin of a cancerous region (“Margin”) or from a distal non-cancerous site (“Normal”).

This analysis was intended to shed light on whether there are differences between the four separate gene expression variables, FAS, USP2a, pAKT and NPY on samples taken from the margins of cancerous regions and non-cancerous samples. The data are shown in Table 7.

TABLE 7
Least Square Means for Gene expression by Site
Margin v. Normal

| | Least | | | | | |
|---|---|---|---|---|---|---|
| | Sq | Std | | | | |
| Site | Mean | Error | Mean | Difference | 95% CI range | p-Value |
| | FAS Expression | | | | | |
| Margin | 0.796 | 0.188 | 0.777 | | | |
| Normal | −0.012 | 0.148 | −0.000 | | | |
| | | | | 0.808 | 0.296 to 1.319 | 0.003 |
| | USP2a Expression | | | | | |
| Margin | 1.742 | 0.331 | 1.538 | | | |
| Normal | 0.718 | 0.262 | 0.850 | | | |
| | | | | 1.025 | 0.122 to 1.927 | 0.0274 |
| | pAKT Expression | | | | | |
| Margin | 0.992 | 0.231 | 0.846 | | | |
| Normal | 0.510 | 0.182 | 0.605 | | | |
| | | | | — | — | 0.1286 |
| | NPY Expression | | | | | |
| Margin | 1.199 | 0.264 | 1.250 | | | |
| Normal | 0.901 | 0.201 | 0.870 | | | |
| | | | | — | — | 0.3939 |

The data reveal that for sites at the margin, FAS, USP2a and pAKT levels are higher than in normal tissue. However, NPY levels are nearly equal at both the margin and in normal non-cancerous sites. For USP2a, the Age variable shows a slightly significant contribution with p=0.0845. For pAKT and NPY, the Age variable show significant contribution with p=0.0774 and p=0.5449, respectively. The Age range found to be significant was between 50-75 years.

### Example 10

### FAS as Prognostic Indicator in Prostate Cancer

Expanding on preliminary findings of the 90-patient cohort reported in PCT Publication PCT/US2010/046773, published Mar. 17, 2011, the contents of which are incorporated herein by reference in their entirety, disclosed here are findings relating to methods of prognosis based on regression and survival times. It has been discovered that there exists a predictive relationship between FAS and degree of regression and clinical survival.

Biopsy specimens from ninety patients diagnosed with prostate cancer (PCa) were prepared and analyzed as described below. All patients had been treated by androgen ablation.

Tissue microarrays (TMAs) were prepared as described in US 2008/0206777 A1, entitled “Gene and protein expression profiles associated with the therapeutic efficacy of EGFR-TK inhibitors,” the contents of which are incorporated herein by reference in their entirety. In addition to the 90 prostate cancer specimens, TMAs containing normal prostate tissue, benign prostatic hyperplasia (BPH) and normal (non-cancerous) tissues were prepared and analyzed.

FAS expression was determined by immunohistochemistry (IHC) according to the method described in US 2008/0206777 A1, using MAb D (generated with peptide 4; SEQ ID No: 4; ATCC Deposit PTA-10801) as the detection (primary) antibody. The detection antibody was visualized using a biotinylated link antibody and streptavidin-HRP as described in US 2008/0206777 A1. The results for the 90 prostate cancer samples are shown in Table 8 below.

In the table, FAS (SEQ ID NO. 6), USP2a (SEQ ID NO. 7), NPY (SEQ ID NO. 9), and AMACR (alpha-methylacyl-CoA racemase, nuclear gene encoding mitochondrial protein, transcript variant 1, OR AMACR IA; GenBank NM_{—}014324; SEQ ID NO 10) expression are represented on a grade scale of 0, trace, 1, 2 or 3 where 0 represents no expression detected. It should be noted that zero or no expression was set equivalent to baseline expression of the gene in normal tissue. Hence a grade or level of 0 means expression was equivalent to normal tissue expression of the gene in question. Trace means that more than 0 but less than 1% over normal tissue expression was detected, 1 means that 1-25% expression over normal tissue was detected, 2 means that 25-75% expression over normal tissue was detected, and 3 means that 75-100% expression over normal tissue was detected as compared to control. The results for the TE-30 array and the Normal Prostate/BPH Screening Array were negative for FAS expression.

TABLE 8
Clinical and pathological data on 90 cases of treated (androgen
ablated) prostate cancer

| Pt | | G- | Pre- | | | | | | | TNM | |
|---|---|---|---|---|---|---|---|---|---|---|---|
| No. | Age | score | PSA | FAS | USP2a | AMACR | NPY | MT | DR | 2002 | S |
| 1 | 69 | 3 + 4 = 7 | 8.7 | 3 | 3 | 1 | 3 | 3 | Poor | pT3aN0R0 | 1 |
| 2 | 70 | 3 + 3 = 6 | 7.0 | 3 | 3 | 1 | 2 | 1 | Poor | pT3aNXR0 | 1 |
| 3 | 72 | 2 + 3 = 5 | 21.0 | 1 | 2 | 1 | 0 | 6 | Excellent | pT2cN0R0 | +5 |
| 4 | 68 | 2 + 2 = 4 | 14.0 | 3 | 2 | 1 | 3 | 7 | Poor | pT2cNXR0 | 2 |
| 5 | 60 | 2 + 3 = 5 | 24.0 | 0 | 1 | 0 | 2 | 3 | Good | pT2cNXR0 | 4 |
| 6 | 68 | 3 + 3 = 6 | + | 2 | 2 | 2 | 3 | 3 | Poor | pT3bN0R1 | 2 |
| 7 | 58 | 3 + 3 = 6 | 10.2 | 2 | 3 | 2 | 3 | 3 | Poor | pT3aNXR1 | 2 |
| 8 | 57 | 3 + 3 = 6 | 9.8 | 3 | 3 | 1 | 3 | 3 | Poor | pT3aNXR0 | 3 |
| 9 | 70 | 3 + 3 = 6 | 5.2 | 2 | 3 | 0 | 3 | 2 | Poor | pT2cN0R1 | 1 |
| 10 | 64 | 3 + 3 = 6 | 2.6 | 1 | 2 | 1 | 1 | 3 | Good | pT2cNXR1 | 1 |
| 11 | 65 | 3 + 3 = 6 | 5.0 | 3 | 3 | 1 | 3 | 5 | Poor | pT3aN0R0 | 1 |
| 12 | 66 | 4 + 3 = 7 | 6.0 | 3 | 3 | 1 | 3 | 5 | Poor | pT3aN0R1 | 2 |
| 13 | 61 | 3 + 3 = 6 | 7.6 | 1 | 2 | 1 | 1 | 6 | Good | pT3aN0R0 | +5 |
| 14 | 68 | 3 + 4 = 7 | 9.7 | 1 | 2 | 1 | 1 | 3 | Good | pT2cN0R1 | 4 |
| 15 | 67 | 3 + 3 = 6 | 9.4 | 3 | 3 | 2 | 3 | 6 | Poor | pT3bN0R0 | 2 |
| 16 | 57 | 2 + 3 = 5 | 19.0 | 3 | 2 | 1 | 3 | 2 | Good | pT2cN0R0 | 3 |
| 17 | 67 | 3 + 3 = 6 | 9.5 | 3 | 3 | 1 | 3 | 7 | Poor | pT3bN1R0 | 2 |
| 18 | 57 | 3 + 4 = 7 | 34.0 | 2 | 3 | 2 | 2 | 4 | Poor | pT3bNXR1 | 1 |
| 19 | 58 | 3 + 3 = 6 | 18.0 | 1 | 1 | 3 | 1 | 11 | Excellent | pT3aN0R0 | 5 |
| 20 | 72 | 3 + 3 = 6 | 6.5 | 3 | 3 | 1 | 3 | 2 | Poor | pT3aN0R0 | 2 |
| 21 | 70 | 3 + 3 = 6 | 8.7 | 3 | 3 | 1 | 3 | 3 | Poor | pT3aN0R1 | 2 |
| 22 | 65 | 4 + 3 = 7 | 16.3 | 3 | 3 | 1 | 3 | 5 | Poor | pT2cN0R1 | 1 |
| 23 | 68 | 3 + 4 = 7 | 41.7 | 0 | 0 | 3 | 0 | 6 | Excellent | pT4N0R1 | +5 |
| 24 | 62 | 3 + 3 = 6 | 13.0 | 3 | 3 | 1 | 3 | 3 | Poor | pT3aN0R0 | 1 |
| 25 | 64 | 2 + 3 = 5 | 2.4 | 3 | 3 | 1 | 3 | 1 | Poor | pT2bN0R0 | 1 |
| 26 | 78 | 3 + 4 = 7 | 13.4 | 1 | 1 | 3 | 1 | 2 | Excellent | pT2cN0R0 | 5 |
| 27 | 56 | 3 + 4 = 7 | 17.1 | 1 | 3 | 1 | 1 | 24 | Good | pT3bN0R1 | 5 |
| 28 | 65 | 3 + 3 = 6 | 16.0 | 2 | 3 | 2 | 3 | 4 | Poor | pT3bN0R0 | 3 |
| 29 | 72 | 3 + 3 = 6 | 19.0 | 2 | 3 | 2 | 3 | 2 | Poor | pT3aN0R0 | 2 |
| 30 | 68 | 3 + 5 = 8 | 2.15 | 2 | 3 | 2 | 3 | 1 | Poor | pT3aN0R0 | 2 |
| 31 | 66 | 4 + 3 = 7 | 12.0 | 3 | 3 | 1 | 3 | 12 | Poor | pT3bN1R0 | 2 |
| 32 | 65 | 3 + 3 = 6 | 2.4 | 3 | 3 | 1 | 3 | 4 | Poor | pT3bN0R1 | 2 |
| 33 | 69 | 3 + 3 = 6 | 7.0 | 3 | 3 | 1 | 3 | 1 | Poor | pT2aNXR0 | 1 |
| 34 | 68 | 3 + 3 = 6 | 17.5 | 0 | 2 | 0 | 0 | 5 | Good | pT2cN0R1 | 5 |
| 35 | 67 | 3 + 4 = 7 | 7.3 | 3 | 3 | 1 | 3 | 6 | Poor | pT3aNXR1 | 1 |
| 36 | 53 | 2 + 2 = 4 | 3.7 | 1 | 2 | 2 | 1 | 3 | Good | pT2cN0R0 | 5 |
| 37 | 66 | 3 + 3 = 6 | 10.6 | 1 | 3 | 1 | 3 | 2 | Poor | pT2cN0R0 | 3 |
| 38 | 74 | 3 + 3 = 6 | 15.9 | 1 | 2 | 1 | 3 | 2 | Poor | pT2cN0R0 | 3 |
| 39 | 69 | 4 + 4 = 8 | 31.0 | 0 | 2 | 3 | 0 | 6 | Good | pT2cN0R0 | 5 |
| 40 | 70 | 3 + 3 = 6 | 7.3 | 3 | 3 | 1 | 3 | 3 | Poor | pT3bN0R1 | 1 |
| 41 | 59 | 4 + 4 = 8 | 27.0 | 3 | 3 | 1 | 3 | 2 | Poor | pT3aN0R0 | .5 |
| 42 | 67 | 3 + 4 = 7 | 16.5 | 3 | 3 | 1 | 3 | 1 | Poor | PT2cNXR0 | 1 |
| 43 | 62 | 3 + 3 = 6 | 5.4 | 1 | 2 | 3 | 1 | 2 | Good | pT3aNXR1 | 5 |
| 44 | 69 | 4 + 4 = 8 | 8.2 | 3 | 3 | 1 | 3 | 5 | Poor | PT2cN0R1 | 1 |
| 45 | 73 | 3 + 3 = 6 | 1.8 | 3 | 3 | 1 | 3 | 2 | Poor | pT3aNXR0 | 1 |
| 46 | 59 | 3 + 4 = 7 | 9.3 | 3 | 3 | 1 | 2 | 6 | Poor | pT2aN0R0 | 1 |
| 47 | 69 | 3 + 3 = 6 | 12.6 | 3 | 3 | 1 | 2 | 3 | Poor | pT2cN0R0 | 1 |
| 48 | 66 | 4 + 3 = 7 | 13.5 | 3 | 3 | 1 | 3 | 6 | Poor | pT2aN0R0 | 1 |
| 49 | 50 | 4 + 3 = 7 | 101.0 | 3 | 3 | 1 | 3 | 4 | Poor | PT4N1R1 | 1 |
| 50 | 53 | 3 + 3 = 6 | 10.0 | 3 | 3 | 1 | 3 | 1 | Poor | pT2aN0R1 | 1 |
| 51 | 70 | 4 + 4 = 8 | 10.9 | 3 | 3 | 1 | 3 | 3 | Poor | pT2aN0R0 | 1 |
| 52 | 55 | 3 + 3 = 6 | 9.7 | 3 | 2 | 3 | 3 | 4 | Good | pT2cN0R0 | 2 |
| 53 | 61 | 3 + 3 = 6 | 6.4 | 3 | 3 | 1 | 3 | 3 | Poor | pT3bN1R1 | 2 |
| 54 | 74 | 3 + 3 = 6 | 28.0 | 0 | 0 | 0 | 0 | 6 | Excellent | pT3bN0R0 | +5 |
| 55 | 71 | 3 + 4 = 7 | 8.3 | +3 | 2 | 2 | 2 | 3 | Good | pT3aN0R0 | 4 |
| 56 | 71 | 3 + 3 = 6 | 17.0 | +3 | 3 | 0 | 3 | 1 | Poor | pT2cN0R1 | 1 |
| 57 | 67 | 3 + 3 = 6 | 2.0 | 1 | 1 | 1 | 1 | 5 | Excellent | pT2aN0R0 | 5 |
| 58 | 62 | 3 + 3 = 6 | 16.6 | 1 | 2 | 1 | 1 | 3 | Good | pT2cN0R0 | 4 |
| 59 | 69 | 3 + 3 = 6 | 6.31 | 3 | 3 | 0 | 3 | 1 | Poor | pT2cNXR0 | 1 |
| 60 | 72 | 3 + 3 = 6 | 11.8 | 3 | 3 | 1 | 3 | 2 | Poor | pT2cN0R0 | 1 |
| 61 | 65 | 3 + 3 = 6 | 5.2 | 3 | 3 | 0 | 3 | 2 | Poor | pT2cN0R0 | 1 |
| 62 | 66 | 3 + 4 = 7 | 7.4 | 3 | 3 | 1 | 3 | 1 | Poor | pT3bN0R1 | 2 |
| 63 | 66 | 4 + 3 = 7 | 13.5 | 0 | 1 | 3 | 0 | 3 | Good | pT3bN0R0 | 5 |
| 64 | 74 | 3 + 3 = 6 | 12.8 | 0 | 1 | 3 | 0 | 4 | Excellent | pT2aNXR0 | +5 |
| 65 | 66 | 3 + 3 = 6 | 9.8 | 3 | 3 | 1 | 3 | 2 | Poor | pT3bN0R0 | 1 |
| 66 | 66 | 3 + 3 = 6 | 8.1 | 1 | 2 | 3 | 2 | 2 | Good | pT2cNXR1 | 5 |
| 67 | 64 | 3 + 3 = 6 | 1.7 | 1 | 2 | 1 | 1 | 7 | Excellent | pT2aNXR0 | 5 |
| 68 | 69 | 3 + 4 = 7 | 5.0 | 1 | 2 | 1 | 1 | 3 | Good | pT2cN0R0 | 3 |
| 69 | 54 | 3 + 3 = 6 | 11.8 | 3 | 2 | 1 | 3 | 3 | Good | PT3bNXR1 | 2 |
| 70 | 69 | 3 + 3 = 6 | 6.2 | 2 | 2 | 2 | 2 | 3 | Good | PT3aN0R0 | 2 |
| 71 | 63 | 3 + 4 = 7 | 8.6 | 2 | 2 | 2 | 2 | 3 | Good | pT2cNXR0 | 3 |
| 72 | 59 | 3 + 4 = 7 | 12.2 | 3 | 3 | 1 | 3 | 3 | Poor | pT2aN0R1 | 1 |
| 73 | 65 | 4 + 3 = 7 | 4.8 | 2 | 1 | 2 | 2 | 2 | Good | pT2aN0R0 | 1 |
| 74 | 64 | 3 + 3 = 6 | 7.6 | 3 | 3 | 1 | 3 | 1 | Poor | pT2aN0R0 | 3 |
| 75 | 72 | 3 + 3 = 6 | 8.4 | 3 | 3 | 1 | 3 | 2 | Poor | pT2cN0R1 | 1 |
| 76 | 68 | 3 + 4 = 7 | 7.8 | 3 | 3 | 1 | 3 | 3 | Poor | pT2bN1R0 | 1 |
| 77 | 65 | 3 + 3 = 6 | 24.0 | 3 | 1 | 1 | 3 | 5 | Good | pT2bN0R0 | 5 |
| 78 | 65 | 3 + 3 = 6 | 6.4 | 3 | 3 | 1 | 3 | 6 | Poor | pT3aNXR0 | 3 |
| 79 | 71 | 3 + 4 = 7 | 9.6 | 1 | 1 | 1 | 1 | 2 | Poor | pT3aN0R1 | 3 |
| 80 | 62 | 4 + 5 = 9 | 27.0 | 1 | 0 | 1 | 1 | 4 | Excellent | pT2aN0R0 | 5 |
| 81 | 71 | 3 + 3 = 6 | 11.6 | 2 | 2 | 2 | 2 | 3 | Good | pT2aN0R0 | 2 |
| 82 | 67 | 4 + 3 = 7 | 25.0 | 3 | 3 | 1 | 3 | 1 | Poor | pT3bNXR1 | 1 |
| 83 | 67 | 3 + 3 = 6 | 22.9 | 3 | 3 | 1 | 3 | 9 | Poor | pT3bN1R1 | 1 |
| 84 | 72 | 3 + 4 = 7 | 7.4 | 0 | 0 | 0 | 0 | 3 | Excellent | pT2bN0R0 | 5 |
| 85 | 71 | 4 + 3 = 7 | 1.3 | 0 | 0 | 0 | 0 | 4 | Excellent | PT3bN0R1 | 5 |
| 86 | 69 | 4 + 3 = 7 | 4.8 | 3 | 3 | 1 | 3 | 3 | Poor | pT3aN0R1 | 1 |
| 87 | 68 | 4 + 3 = 7 | 9.0 | +3 | 3 | + | + | 1 | Poor | pT3bN0R0 | 1 |
| 88 | 70 | 3 + 3 = 6 | 6.1 | 1 | 2 | 1 | 1 | 3 | Good | pT3aN0R0 | UNK |
| 89 | 71 | 4 + 3 = 7 | 20.4 | 3 | 3 | 1 | 3 | 1 | Poor | pT3aN0R0 | 1 |
| 90 | 65 | 3 + 3 = 6 | 5.8 | 3 | 3 | 1 | 3 | 3 | Poor | pT3aNXR1 | 1 |

In the table,
“Pt” stands for patient and each has been assigned an arbitrary number;
Age is the respective patient's age in years;
“G-score” refers to the split Gleason score from each biopsy;
“Pre-PSA” refers to the patient's pre-therapy total PSA levels in ng/mL (pre-PSA grade 0 = 0-5 ng/ml;
1 = 6-60 ng/ml;
2 = 61-100 ng/ml; and
3 = 101-150 ng/ml); “FAS” refers to the gene
Fatty Acid synthase;
“USP2a” refers to the gene ubiquitin specific peptidase 2;
“AMACR” refers to the gene alpha-methylacyl-CoA racemase;
“NPY” refers to the gene Neuropeptide Y;
“MT” refers to the number of months each patient has had therapy;
“DR” is degree of regression;
TNM2002” refers to tumor grade set out as Tumor, Node, Metastasis grading system;
“S” refers to patient Survival (in years) and
“UNK” represents unknown.

### Example 11

### FAS and Cancer Regression

The relationship between FAS and degree of regression were of primary interest and, as such, patient survival and regression data were analyzed using the SAS statistical package, JMP, version 8 (Cary, N.C.). The data consisted of 90 observations which are detailed in Table 8 of Example 10. 51 observations had a FAS value of 3, 10 with FAS=2, 19 with FAS=1, 9 with FAS=0. One biopsy (Patient 87) did not have a FAS value. Degree of regression was measured on a three grade scale of Poor-Good-Excellent, where Poor means the patient died between 1-2 years, Good means the patient is alive with the disease within 5 years and Excellent means greater than 5 years of survival. The highest frequency shows “Poor” degree of regression with FAS=3.

TABLE 9
DOR Frequencies

| | Degree of regression | | | |
|---|---|---|---|---|
| Frequencies | (3 grade system) | | | |
| FAS | Poor | Good | Excellent | Responses |
| missing | 1 | | | 1 |
| 0 | | 4 | 5 | 9 |
| 1 | 3 | 10 | 6 | 19 |
| 2 | 6 | 4 | | 10 |
| 3 | 46 | 5 | 21/24 | 51 |
| -All- | 56 | 23 | 11 | 90 |

In this situation, Degree of regression is the categorical response variable, and FAS is the categorical sample variable. The test for marginal homogeneity tests that the response probabilities are the same across FAS levels. This analysis is similar to a chi-square (or likelihood ratio) test for independence. Both types of test show a strong relationship between FAS and degree of regression with p-values below 0.0001. The test response homogeneity is shown in Table 10.

TABLE 10
Test Response Homogeneity

| Test | ChiSquare | Prob > ChiSq |
|---|---|---|
| Likelihood Ratio | 65.8534 | <.0001* |
| Pearson | 58.7950 | <.0001* |

This indicates that higher FAS numbers are associated with a poorer degree of regression.

Further analysis was performed using FAS as a numerical X-variable (with Degree of regression a categorical Y-variable). It should be understood that a categorical Y-variable with a numerical X-variable leads to Logistic Regression. By assuming that FAS is a numerical variable, we assume that a value FAS=2 is about twice as strong as FAS=1. The advantage of making this assumption is that, based on log odds results, it allows determination of how strong the separation between the response categories are.

The analysis revealed that the relationship between Degree of regression and FAS is highly significant (p-value<0.0001). This has already been shown with the Test of Homogeneity.

However, with logistic regression the following additional statements can be made:

Log Odds of Poor to Excellent significant with p<0.0001;

Log Odds of Poor to Good significant with p<0.0001, and

Log Odds of Good to Excellent significant with p=0.0243.

The estimated probabilities of classification into a degree of regression according to FAS value are shown in the Table 11.

TABLE 11
Estimated probabilities of regression

| | Degree of | | | |
|---|---|---|---|---|
| | regression = Y | | | |
| FAS = X | Poor | Good | Excellent | n |
| 0 | 0.015 | 0.359 | 0.627 | 9 |
| 1 | 0.151 | 0.594 | 0.255 | 19 |
| 2 | 0.587 | 0.374 | 0.039 | 10 |
| 3 | 0.905 | 0.093 | 0.002 | 51 |

The data in the table indicate that the estimated probability of a “Poor” degree of regression depends on FAS. In the columns marked “Poor”, when FAS = 0, the probability is 0.015; when FAS = 1, the probability is 0.151; when FAS = 2, the probability is 0.587; when FAS = 3, the probability is 0.905.

A cross-classification of the actual degree of regression outcome with the most likely outcome predicted by logistics regression shows that FAS predicts Poor results very well. These data are shown in Table 12.

TABLE 12
Predictive power of FAS

| | Most Likely Degree of | | | |
|---|---|---|---|---|
| | regression (Logistic Reg) | | | |
| Actual Degree of | | | Predicted | |
| regression | Predicted Poor | Predicted Good | Excellent | Total |
| Actual Poor | 52 | 3 | 0 | 55 |
| Actual Good | 9 | 10 | 4 | 23 |
| Actual Excellent | 0 | 6 | 5 | 11 |
| Total | 61 | 19 | 9 | 89 |

The ROC (Receiver Operating Characteristic) curves confirm these numerical results and are shown in Table 13.

TABLE 13
Receiver Operating Characteristic

| Degree of regression (3 grade system) | Area |
|---|---|
| Excellent | 0.9149 |
| Good | 0.7510 |
| Poor | 0.8888 |

In the ideal case all three categories would have an associated area of 1. In the worst case they would cover the diagonal of a plot of sensitivity (y axis) v. specificity (x axis). The two extreme categories: Excellent and Poor have Areas 0.9149 and 0.8888 respectively. The middle category, Good, where misclassifications can go either to Poor or Excellent, had the lowest value as would be expected.

### Example 12

### FAS and Clinical Survival

Based on the data of 90 patients (outlined in Example 10), the following variables were analyzed: Gleason, Pre-therapy PSA (ng/ml), FAS (0-3 grade system of level of expression), Months of therapy as the predictor variables. Survival Time (years) was the predicted variable. Five patients survived 5 years and these were identified by the variable Censor. The main analysis involved fitting a parametric regression model of the first four variables to explain the survival times with 5 censored observations.

By far the most significant variable in explaining survival time was FAS. On the Effect Likelihood Ratio Tests, the Chi-Square statistic of this variable was over 12 times larger than the next most significant variable. Gleason (p=0.0157) and Months of therapy (p=0.0097) were also statistically significant. Pre-therapy PSA was not at all significant (p=0.6281). Given the other three variables, PSA offered nothing in terms of explaining survival (failure) time. Consequently, based on the data, it appears that FAS is by far the strongest indicator of time to survival or failure, while Pre-therapy PSA is virtually of no value.

It is important to note that the parametric survival fit had estimated regression coefficients that were only negligibly correlated with each other. This simplifies the interpretation, and allows for independent statements about each variable. The Cox Proportional Hazard model yield essentially similar results, except that Gleason and Months of therapy were not significant at σ=0.05. With the Cox model FAS is the only significant and still an outstanding variable to explain survival time.

It is important to note that the values of the four predictor variables were not spread out according to an optimum allocation scheme (Given the data sources, this may not be possible). 86 pre-therapy PSA values were below 30. A greater spread in PSA values (especially by including more of the larger values) might have an effect on future results. Similarly, 78 Gleason values are either 6 or 7; two are 4; four are 5, and five are 8. Including more 4, 5 and 8 scores, might enhance the significance of Gleason. Lastly, 29 of 90 observations had Gleason=6 and FAS=3. But also, 52 of FAS were 3, and yet this variable is very significant.

### Gleason Score Analysis

The most frequent Gleason value is 6 with 49 observations, followed by 7 with 29 observations. The cross tabulation of FAS with Gleason shows that 29 (about a third) observations fall into the joint category with Gleason=6 and FAS=3). The summary is provided in Table 14.

TABLE 14
Cross-tabulation of FAS versus Gleason scores
(Values in the body of the table are frequencies).

| | | | | | | | | | Total # | |
|---|---|---|---|---|---|---|---|---|---|---|
| | | | | | | | | | FAS | % of |
| | G | | | | | | | | for G- | G- |
| | Scores | Gleason | | | | | | Total # | score | score |
| | (4-9) | 4 | 5 | 6 | 7 | 8 | 9 | FAS | 5-7 | 5-7 |
| FAS | 0 | 0 | 1 | 3 | 4 | 1 | 0 | 9 | 8 | 88 |
| (0-3 | 1 | 1 | 1 | 11 | 5 | 0 | 1 | 19 | 17 | 89 |
| grade) | 2 | 0 | 0 | 6 | 3 | 1 | 0 | 10 | 9 | 90 |
| | 3 | 1 | 2 | 29 | 17 | 3 | 0 | 52 | 48 | 92 |
| | Total # | 2 | 4 | 49 | 29 | 5 | 1 | 90 | | |
| | Gleason | | | | | | | | | |

It can be seen from the table that most of the data (greater than 88%) fall within the range of Gleason score 5-7. Hence FAS grade in combination with Gleason scores of between 5 and 7 may be used as a combination predictor of clinical survival, at least post surgically.

### Parametric Survival Fit

The data were then fit to an equation for predicting survival. The equation fits the Log (Survival Time) as a Y-variable with Gleason, Pre-therapy PSA, FAS and Months of therapy as X-variables. The variable “censor” is used to identify those patients who survived 5 years. The resulting equation is:

Log(Survival Time (years))=2.52502+−0.13595*Gleason+−0.00180*Pre-therapy PSA+−0.49465*FAS+0.03869*Months of therapy.

### The Effect Likelihood Ratio (Effect LR)

In order to assign a separate p-value for each term in the equation, an effect likelihood ratio was calculated. Table 15 shows the results. A p-value BELOW 0.05 is taken to indicate an effect that is significantly different from 0. Such a variable would be considered to influence survival time. The Effect LR tests show that all but Pre-therapy PSA are significant. Pre-therapy PSA has a p-value=0.6281 and does not affect survival time. Of the three significant variables, FAS has the largest ChiSquare test statistics (85.29 versus 5.84 for Gleason and 6.68 for Months of therapy). This suggests that FAS is not only the most significant variable, but is overwhelmingly more significant, relative to the other variables.

TABLE 15
Effect Likelihood Ratio Tests

| | | L-R | | |
|---|---|---|---|---|
| Source | DF | ChiSquare | Prob > ChiSq | |
| Gleason | 1 | 5.84 | 0.0157 | significant |
| Pre-therapy PSA (ng/ml) | 1 | 0.23 | 0.6281 | Not |
| | | | | significant |
| FAS (0-3 grade system) | 1 | 85.29 | <0.0001 | significant |
| Months of therapy | 1 | 6.68 | 0.0097 | significant |

The Parameter Estimates are all reasonable and are outlined in Table 16. FAS and Gleason have negative coefficient, indicating that an increase in value reduces survival time. Months of therapy have a positive coefficient, and this means that longer therapy increases survival time. The coefficient of PSA is ignored since it is not significant. The 95% confidence interval for the coefficient of PSA, from −0.0091 to +0.0056, includes 0. This is another indicator that it is not significant. The 95% confidence intervals of all the other coefficients do not include 0 and these shows in a different way that they are significantly different from 0. The parameter σ is the scale parameter and does enter the equation directly.

TABLE 16
Parameter Estimates of Survival Model (LogNormal)

| Term | Estimate | Std Error | Lower CL | Upper CL |
|---|---|---|---|---|
| Intercept | 2.5250 | 0.3724 | 1.7912 | 3.2681 |
| Gleason | −0.1360 | 0.0556 | −0.2466 | −0.0262 |
| Pre-therapy PSA (ng/ml) | −0.0018 | 0.0037 | −0.0091 | 0.0056 |
| FAS (0-3 grade system) | −0.4946 | 0.0427 | −0.5802 | −0.4109 |
| Months of therapy | 0.0387 | 0.0147 | 0.0096 | 0.0679 |
| σ | 0.4086 | 0.0315 | 0.3538 | 0.4792 |

The Lognormal was chosen because the residual plot suggested that the assumptions are best satisfied by this model. Weibull and LogLogistic were also tried. Whereas the Weibull did not fit the data as well, the LogLogistic had a very good fit, because it is similar to the LogNormal. However, LogNormal model is more easily understood.

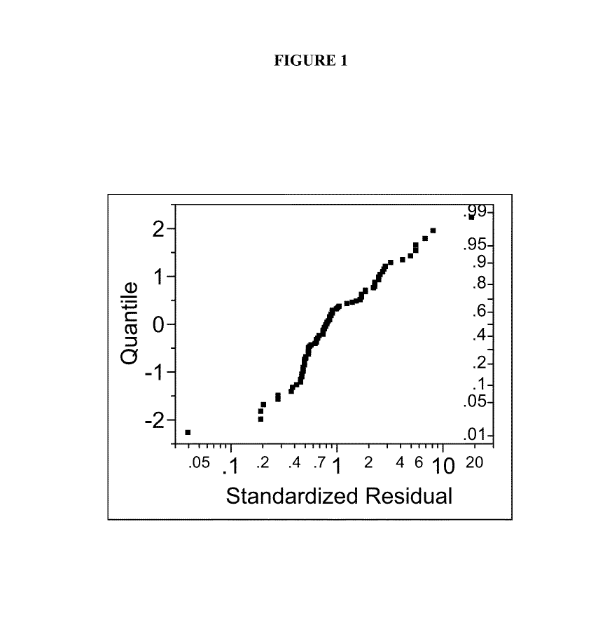

The residual quantile plot of the equation (FIG. 1) shows that the residuals (=observed−predicted) are well-behaved. The line is not quite straight line, but it does not bend or curve in one or the other direction as the Weibull and even the LogLogistic.

### Distribution Profiler

It is evident from the data that FAS is the most important variable in this equation. Another way to visualize the effect of FAS on the probability of failure (1−probability of survival) can be seen from different settings of the Distribution Profiler.

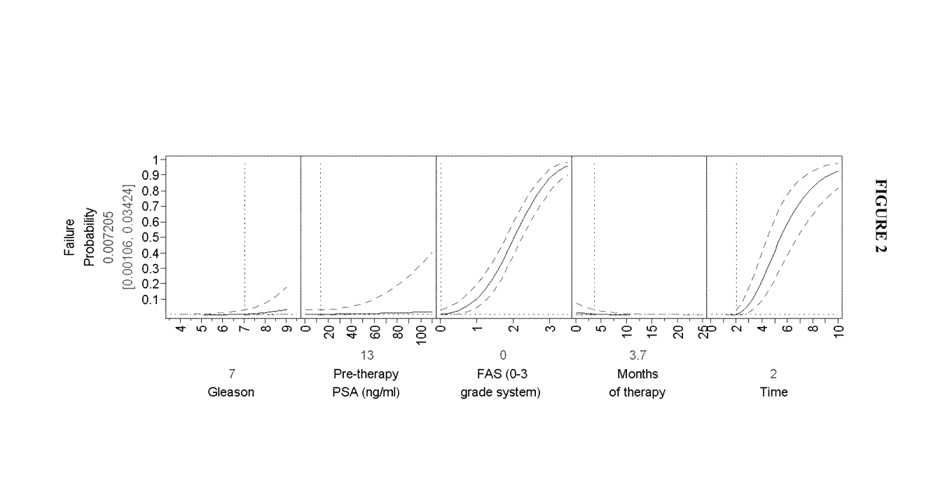

In this example, the profiler consists of 5 graphs plotting the Failure Probability (y-axis) versus Gleason, Pre-therapy PSA, FAS, Months of therapy and Time (all on the x-axis). Four Distribution Profilers are shown in FIGS. 2-5. In the plots, certain values have been held constant. These are: Gleason=7, PSA=13, Months of therapy=3.7, and Time (of failure)=2. These values are the rounded values nearest their mean.

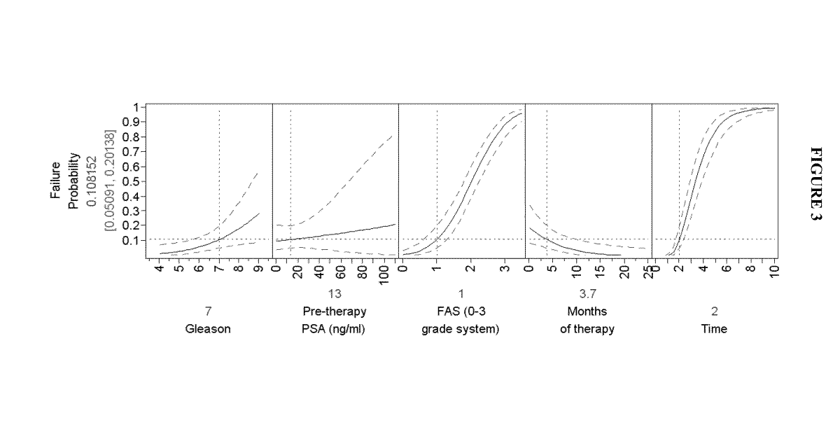

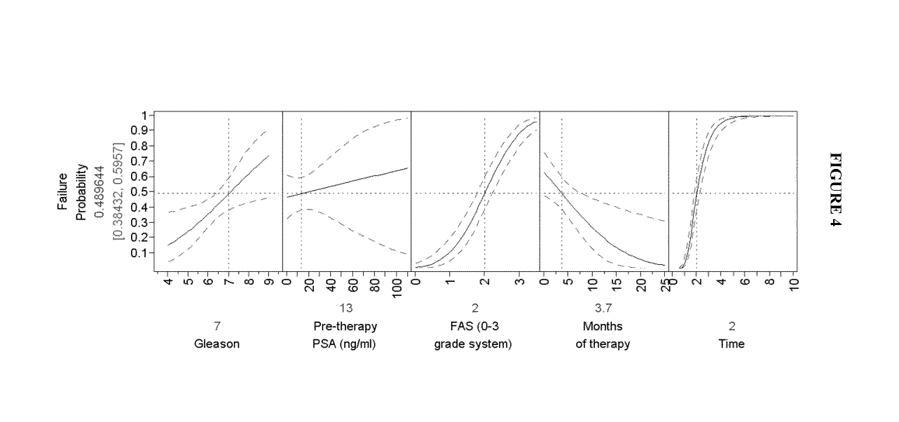

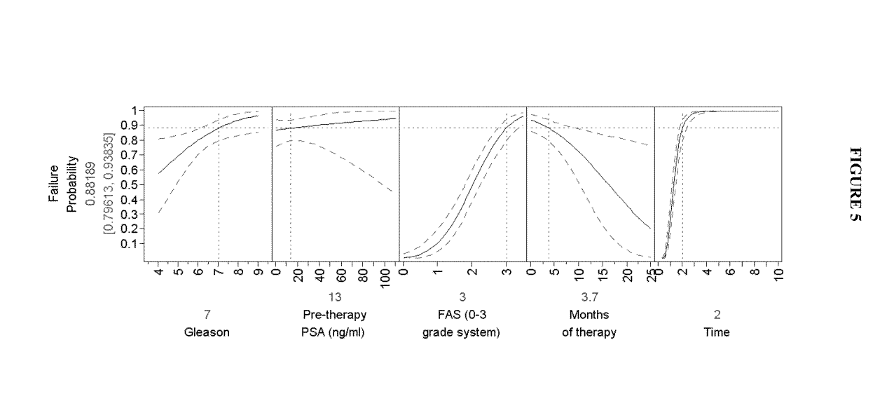

In each profiler we chose a different FAS value: FAS=0 (FIG. 2), FAS=1 (FIG. 3), FAS=2 (FIG. 4) and FAS=3 (FIG. 5). One of skill would be able to construct more profilers by choosing different values for the other variables.

Distribution Profiler with FAS=0

The profiler for a patient with FAS=0 and with Gleason=7, PSA=13, Months of therapy=3.7, and Time (of failure)=2, estimates the probability of failure as 0.007205 (on the left axis) with a 95% confidence interval that it is between 0.00106 and 0.03424. The solid curves of Gleason, PSA, Months show no effect, meaning that with FAS=0, the probability of failure mostly depends on Time.

### Distribution Profiler With FAS=1

The profiler for a patient with FAS=1 and as before with Gleason=7, PSA=13, Months of therapy=3.7, and Time (of failure)=2, estimates the probability of failure as 0.108152 (on the left axis) with a 95% confidence interval that it is between 0.05091 and 0.20138. The solid curves of Gleason is curved upward and this shows that an increase from the current setting Gleason=7 results in an increase in failure probability. PSA is not significant, but points in the proper direction. As Months of therapy increases, the failure probability decreases. The change in the (S-shaped curve) of Time (versus Failure Probability) is much steeper for FAS=1 than for FAS=0.

### Distribution Profiler With FAS=2

The profiler for a patient with FAS=2 and as before with Gleason=7, PSA=13, Months of therapy=3.7, and Time (of failure)=2, estimates the probability of failure as 0.489644 (on the left axis) with a 95% confidence interval that it is between 0.38432 and 0.5957. For this setting almost half the patients are estimated to die by year 2. The solid curves of Gleason is strongly curved upward and this shows that an increase from the current setting Gleason=7 results in an even steeper increase in failure probability than that for FAS=1. PSA is not significant, but does point is in the proper direction. As Months of therapy increases, the failure probability decreases. The change in the (S-shaped curve) of Time (versus Failure Probability) is again much steeper for FAS=2 than for either FAS=0 or 1.

### Distribution Profiler With FAS=3

The profiler for a patient with FAS=3 and as before with Gleason=7, PSA=13, Months of therapy=3.7, and Time (of failure)=2, estimates the probability of failure as 0.88189 (on the left axis) with a 95% confidence interval that it is between 0.79613 and 0.93835. For this setting almost 90% of patients are estimated to die by year 2. The solid curves of Gleason is curved upward and this shows that an increase from the current setting Gleason=7 results in an increase in failure probability. PSA is not significant, but does point in the proper direction. As Months of therapy increases, the failure probability decreases as with the other FAS values. The change in the (S-shaped curve) of Time (versus Failure Probability) is steepest for FAS=3. The steepness of the curve reinforces that most patients with FAS=3 will die very quickly unless other measures are taken. It should be noted that, this interpretation rest on the representativeness of the current patient mix.

### Other Models

We have fitted a proportional hazard model to the data as well. Even though the PPH model requires certain assumptions, which this analysis cannot verify, the results are similar. FAS is the strongest variable by far. Gleason and Months of therapy are not significant. PSA is also not significant.

### Example 13

### Relative Predictive Power of Other Indicators

Given the findings surrounding FAS expression, other potential metrics which may be useful indicators were evaluated. These included measurements of gene expression of the USP2a, AMACR and NPY genes. It was desirable to address whether any other gene was just as advantageous as a marker or indicator of clinical management parameters or clinical endpoints as FAS.

The data were analyzed using the SAS statistical package, JMP, version 8 (Cary, N.C.). The raw data consisted of 90 observations which are detailed in Table 8 of Example 10.

Creation of Mosaic plots (visual plots which helps determine if the probability for the various response levels is a function of the x level) showed that USP2a and NPY are closely related to FAS. It was determined that for both plots the largest coverage for FAS where FAS=3, coincides with USP2a=3 and NPY=3. While there were some discrepancies (some FAS=3 are also in USP2a=1 or 2 and also for NPY=2), most outcomes were consistent with the conclusion that these three variables (FAS, USP2a and NPY) result in similar grades. By Mosaic plot, the Gleason score was not related to FAS. By related it is meant that Gleason score cannot substitute for FAS as a predictor, e.g., it is not a substantial stand-alone predictor.

Interestingly, an inverse relationship was identified between the gene AMACR and FAS. That is, where AMACR scores are low, FAS scores are high. This result suggests that AMACR expression may also be an equally valuable indicator but that the values will have an inverse relationship to FAS expression levels.

In order to measure the degree of agreement between any two genes, for example between FAS and USP2a, Kappa coefficients were calculated. When two binary variables are attempts by two individuals to measure the same thing, one can use Cohen's Kappa (often called Kappa) as a measure of agreement between the two individuals. Kappa measures the percentage of data values in the main diagonal of the table and then adjusts these values for the amount of agreement that could be expected due to chance alone. These are shown in Table 17. The higher Kappa coefficient indicates that two values are closely related.

TABLE 17
Degree of Agreement

Degree of
Agreement

| | | Standard |
|---|---|---|
| Comparison | Kappa | Error |
| FAS v. USP2a | 0.412005 | 0.066303 |
| FAS v. AMACR | 0.14604 | 0.06463 |
| FAS v. NPY | 0.734385 | 0.063916 |

It can be seen from that data that FAS and NPY are most closely related as shown by the highest kappa coefficient. FAS and USP2a are also closely related. Consequently, it can be concluded that either USP2a or NPY may be used as a prognostic and/or diagnostic indicator of prostate cancer and be at least as predictive or diagnostic as FAS to the level of 73% or 41%, respectively.

### Example 14

### Contingency Analysis of Degree of Regression by Individual Predictors

In another set of analyses of the data detailed in Table 8 of Example 10 and using the SAS statistical package, IMP, version 8 (Cary, N.C.), the relationship between degrees of regression (DOR) and individual predictors/indicators was investigated. The results are shown in Table 18.

TABLE 18
Degree of Regression (DOR) v. Predictor

| | Relationship Value | | |
|---|---|---|---|
| | ChiSquare | Method of | |
| Comparison | p-value | Analysis | Significance |
| DOR v. Pre-therapy PSA | 0.6696 | Logistic | Not |
| (ng/mL) | | Regression | Significant |
| DOR v. Gleason Score | 0.3145 | Mosaic Plot | Not |
| | | | Significant |
| DOR v. FAS | <0.0001 | Mosaic Plot | Significant |
| DOR v. USP2a | <0.0001 | Mosaic Plot | Significant |
| DOR v. AMACR | <0.0001 | Mosaic Plot | Significant |
| DOR v. NPY | <0.0001 | Mosaic Plot | Significant |
| DOR v. Months of Therapy | 0.0488 | Mosaic Plot | Significant |

From the table it is evident that neither pre-therapy PSA nor Gleason score alone are significantly correlated with degree of cancer regression. However, each of the genes FAS, USP2a, AMACR and NPY showed highly significant relationships with prediction of degree of cancer regression. Thus, any of the individual genes could serve as a marker or endpoint for prognosis of degree of regression.

### Example 15

### Multiple Predictors of Degree of Regression

In an effort to improve prediction of degree of regression, combinations of predictor variables were investigated. Again, the analyses involved the data detailed in Table 8 of Example 10 and used the SAS statistical package, JMP, version 8 (Cary, N.C.). The results are shown in Table 19.

TABLE 19
Combination Predictors

| | ChiSquare |
|---|---|
| Combination | p-value |
| FAS alone | <0.0001 |
| FAS and Gleason | 0.2374 |
| FAS and USP2a | 0.0650 |
| FAS and USP2a and Months of | 0.0397 |
| Therapy | |
| FAS and AMACR | 0.2997 |
| FAS and NPY | 0.0003 |
| USP2a and Months of Therapy | 0.0001 |

The results from an ordinal logistic regression show that FAS was the significant variable (p<0.0001) and that the Gleason Score does not add anything to the analysis (p=0.2374).

Using a combination of FAS and USP2a creates a predictor equation that is significant. However, as an individual variable, FAS is significant only at Alpha=0.10. The RSquare of the equation was 0.676. Adding Months of therapy to the equation increases the RSquare to 0.736, but it also renders FAS non-significant. While, eliminating the three largest values in Months of therapy, renders that variable significant at only Alpha=0.10

Using a combination of FAS and AMACR creates a predictor equation that is significant. However, as an individual variable, with a p=0.2997, AMACR is not significant at Alpha=0.10. The RSquare of the equation was 0.676.

Combining FAS and NPY results in an equation that is not much improved over individual variables. Because both variables are so similar, once NPY is significant, FAS did not explain anything that has not already been explained by NPY.

A model with only USP2a and Months of Therapy keeps a high RSquare (=0.7212) and overall significance. However, the three largest observations tend to increase the importance of Months of therapy. Nevertheless, both predictors were highly significant.

In conclusion, it was determined that USP2a was the strongest predictor of Degree of regression and that FAS and NPY were very similar predictors. Finally, and contrary to accepted paradigms, PSA and Gleason were not significant predictors of degree of regression.

### Example 16

### Differential Expression of FAS and USP2a: Local vs. Metastasis Sites

Analysis of the expression data between FAS and USP2a revealed a pattern of differential expression as between local sites and sites of metastasis distal to the cancer. Plotting these levels, it was revealed that there appears to be a relationship that is predictive of clinical outcome, especially degree of regression.

It has been discovered that USP2a and FAS were found to exhibit a differential pattern of expression that, if examined at certain time points, provides a highly significant and powerful means to predict cancer aggressiveness in the context of degree of regression (DOR). It has been determined that increased USP2a expression leads increased FAS expression. It has also been determined that on FAS increased expression, USP2a levels will drop prior to the drop of FAS levels. This pattern of expression is associated and correlated with aggressiveness of prostate cancer. Consequently, the pattern of expression provides a window in which prognosis may be made with confidence. In one embodiment, USP2a expression is measured and compared to FAS expression. Where USP2a expression exceeds FAS expression but then subsequently drops, a strong indication of aggressiveness can be assumed. This window of prognosis might have otherwise been ignored since low measurements of FAS may have suggested a less aggressive form of cancer, thereby mitigating any risk assessment by a clinician. In other words, prior to the present invention, a low FAS expression level might have misled a clinician not to measure USP2a levels and to have missed a critical diagnosis.

### Example 17

### Specificity of Outcome Predictions: FAS and USP2a

Given the unexpected relationships identified with the USP2a gene expression, further analysis using Logistic models was conducted and data generated for the most likely outcomes based on the three models. The most likely degree of regression outcome (Poor, Good, Excellent) was chosen on the basis of the highest estimated probability for each category. This is, of course, the end result of logistic regression. Degree of regression was measured on a three grade scale of Poor-Good-Excellent, where Poor means the patient died between 1-2 years, Good means the patient is alive with the disease within 5 years and Excellent means greater than 5 years of survival with our without the disease. The analyses here involved the data detailed in Table 8 of Example 10 and used the SAS statistical package, JMP, version 8 (Cary, N.C.). The results of RSquare as a rough indicator are shown in Table 20.

TABLE 20
RSquare values of three logistic models

| Combination | RSquare |
|---|---|
| FAS alone | 0.39 |
| USP2a alone | 0.65 |
| FAS and USP2a; | 0.68 |
| Where individual p-values for | |
| FAS = .065 and USP2a = <0.0001 | |

The data reveal that adding FAS to an equation that already includes USP2a raises the RSquare from 0.65 to 0.68. But adding USP2a to an equation that already includes FAS raises the RSquare from 0.39 to 0.68.

Next, cross tabulation of actual outcomes with predicted outcomes was performed. A perfect model would classify all Poor as Poor, all Good as Good, etc. The data showing the most likely degree of regression for each of : (1) FAS alone, (2) USP2a alone, and (3) FAS plus USP2a are shown in Tables 21-23.

TABLE 21
Contingency Table: DOR v. FAS alone

| Count | Excellent | Good | Poor | Total |
|---|---|---|---|---|
| Poor | 0 | 9 | 53 | 62 |
| Good | 6 | 10 | 3 | 19 |
| Excellent | 5 | 4 | 0 | 9 |
| Actual | 11 | 23 | 56 | 90 |

The column categories represent actual outcomes. The rows represent predicted outcomes using FAS alone. For example in the “Excellent” column with a total of 11 actual “Excellent” outcomes, FAS would classify 6 of those as Good and 5 correctly as Excellent. In the “Good” column with a total of 23 actual “Good” outcomes, FAS would classify 9 as Poor, 10 as Good, and 4 as Excellent. It is shown in Table 22 below that this column is where USP2a is a much better predictor than FAS. In the Poor column with a total of 56 actual “Poor” outcomes, FAS would classify 53 as Poor and 3 as Good. The Degree of Agreement of between actual and predicted outcomes has a Kappa=0.51, which is a fairly high value, but lower than the one when USP2a is used.

TABLE 22
Contingency Table: DOR v. USP2a alone

| Count | Excellent | Good | Poor | Total |
|---|---|---|---|---|
| Poor | 0 | 1 | 52 | 53 |
| Good | 6 | 22 | 4 | 32 |
| Excellent | 5 | 0 | 0 | 5 |
| Actual | 11 | 23 | 56 | 90 |

As above, the column categories represent actual outcomes. The rows represent predicted outcomes using USP2a alone. Here, in the “Excellent” column with a total of 11 actual Excellent outcomes, USP2a would classify 6 of those as Good and 5 correctly as Excellent (the same as with FAS). In the “Good” column with a total of 23 actual Good outcomes, USP2a would classify 1 as Poor, 22 as Good, and 0 as Excellent, much better than with FAS alone. In the “Poor” column with a total of 56 actual Poor outcomes, USP2a would classify 52 as Poor and 4 as Good. The Degree of Agreement of between actual and predicted outcomes has a Kappa=0.77, a very high value, and only slightly lower than when USP2a plus FAS is used.

TABLE 23
Contingency Table: DOR v. FAS plus USP2a

| Count | Excellent | Good | Poor | Total |
|---|---|---|---|---|
| Poor | 0 | 1 | 52 | 53 |
| Good | 3 | 22 | 3 | 28 |
| Excellent | 8 | 0 | 1 | 9 |
| Actual | 11 | 23 | 56 | 90 |

As above, the column categories represent actual outcomes. The rows represent predicted outcomes using USP2a in combination with FAS. The “Excellent” column with a total of 11 actual Excellent outcomes, USP2a would classify 3 of those as good and 8 correctly as Excellent. In this column the combination USP2a+FAS shows the greatest benefit. In the “Good” column with a total of 23 actual Good outcomes, USP2a+FAS would classify 1 as Poor, 22 as Good, and 0 as Excellent, the same as with USP2a alone. In the “Poor” column with a total of 56 actual Poor outcomes, USP2a+FAS would classify 52 as Poor and 3 as Good and 1 as Excellent. The Degree of Agreement of between actual and predicted outcomes has a Kappa=0.84, a very high value, and the highest of the three models.

In conclusion FAS measurements alone are as good in classifying Poor outcomes as USP2a alone or USP2a+FAS combination. However, it was USP2a measurements which were found to be best in classifying Good as Good. (The same holds for USP2a+FAS).

Furthermore, the combination FAS+USP2a presents a striking advantage in classifying Excellent as Excellent. Consequently, clinicians would be well advised to incorporate measurements of USP2a alone and in combination with FAS in assays for prognosis of cancer regression.

For example, on presentation of potential prostate cancer when diagnosis and treatment regimens are critical to define, USP2a should be the first line assay. The levels are as good as FAS at predicting Poor outcomes (death in 1-2yrs) and better at predicting Good outcomes (survival of at least 5 years with disease). Stratification across these two most dire prognoses is of the most importance and a single assay which can delineate possible treatment protocols between the two would be incredibly valuable. USP2a has been shown here to satisfy this requirement. For a second tier assay, it has been determined that measurements of FAS may be added to improve the granularity of prediction of Excellent outcomes. The present invention provides such methods, assays and kits.

### Example 18

### Expression of FAS and Her2/neu in Bone Metastasis

Analysis of the expression of FAS and Her2/neu (a known proto-oncogene) revealed a correlation with expression and poor prognosis in prostate cancer patients. Her2/neu (also known as ErbB-2 or Epidermal growth factor Receptor 2) is a protein found to convey aggressiveness in breast cancers. In the present study, expression of both genes was found to be elevated in bone metastases of prostate cancer patients.

### Example 19

### Production and Characterization of Polyclonal Antisera for USP2a

SPF rabbits (Maine Biotechnology Services, Inc.) were used to generate polyclonal antisera. Forty eight (48) rabbits were used. The polyclonal antibodies are referred to hereinafter as USP2a-1 and USP2a-2 and have been deposited with the ATCC (ATCC deposit numbers ______ and ______, respectively).

Each rabbit was injected with one of the peptides of the present invention as shown in Table 24.

TABLE 24
Injected Peptides

| | |
|---|---|
| USP2a-1 (SEQ ID NO. 11) | LTRPRTYGPSSLLDYDRGRPL |
| USP2a-2 (SEQ ID NO. 12) | GGGKRAESQTRGTERPLGS |

The rabbits were bled, and the resulting antisera were then pooled and affinity purified using the same epitopes against which they had been raised from peptides from USP2a (SEQ ID NO. 7). Affinity purification was carried out according to the following procedure:

### Step 1: Affinity Column Preparation

The immunoaffinity column was prepared by coupling the peptides of SEQ ID NO. 11 or SEQ ID NO. 12 to 1 ml of activated sepharose beads.

### Step 2: Loading of the Antisera

The antisera was loaded at a concentration of 2 μg/mL onto the peptide-sepharose column and incubated 1 hour at 37° C.

### Step 3: Elution

After several washes of the column, the elution of bound antibody was performed using elution buffer containing 0.02% sodium azide. Fractions containing the antibody were pooled and the final concentration of immunopurified antibody was determined by reading the optical density at 280 nm using U.V. spectrophotometer.

### Step 4: ELISA Test of the Immunopurified Antibody

The blocking reagent SeaBlock was loaded into the wells in a NEAT concentration and incubated for 30 minutes at 37° C. After the incubation, four samples of serum (pre-bleed Rb 1, pre-bleed Rb 2, peptide 1 and peptide 2) were added into the wells at 6 different concentrations. The four samples were diluted using 0.15M PBS to concentrations of 1:50, 1:250, 1:1250, 1:6250, 1:31250, and 1:156000. Each of these concentrations of the four serums were added to the wells then incubated at room temperature for 30 minutes. Lastly, a secondary antibody, anti-Rb HRP, (HRP-lot #86569) was diluted to a concentration of 1:10000 using 0.15M PBS with 0.05% Tween20 and incubated at room temperature for 30 minutes. The final concentration of the samples, as shown in Tables 25and 26, was determined by reading the absorbance at 450 nm using the U.V. spectrophotometer.

TABLE 25
Pre-Bleed Rb 1 and 2 Sample Analysis

| | ELISA Reactivity to | ELISA Reactivity to |
|---|---|---|
| Concentration | Antisera Rb1 | Antisera Rb2 |
| 1:50 | 0.33 | 0.34 |
| 1:250 | 0.24 | 0.02 |
| 1:1250 | 0.36 | 0.03 |
| 1:6250 | 0.33 | 0.01 |
| 1:31250 | 0.20 | 0.20 |
| 1:156000 | 0.43 | 0.13 |

TABLE 26
USP2a-1 Sample Analysis

| | ELISA Reactivity to | ELISA Reactivity to |
|---|---|---|
| Concentration | Antisera USP2a Peptide 1 | Antisera USP2a Peptide 2 |
| 1:50 | 0.79 | 0.87 |
| 1:250 | 0.58 | 0.58 |
| 1:1250 | 0.94 | 0.85 |
| 1:6250 | 0.93 | 0.63 |
| 1:31250 | 0.86 | 0.95 |
| 1:156000 | 0.64 | 0.53 |

### Example 20

### Sensitivity and Specificity ROC Curves for Five Year Survival

Using the data from Example 10, the sensitivity and specificity of the 90-patient cohort was classified by a binary variable into Survival (Survive5) and No Survival (NoSurv), depending on their survival time being 5 or more years. The five year survival variable was modeled with four different predictor X variables (FAS, USP2a, AMACR, and NPY) as well as Gleason Score and pre-therapy PSA. The resulting models were used to predict the most likely outcome (again Survival or No Survival). The true outcomes were compared with the predicted outcomes, based on the different models. From the cross-classification, the Sensitivity and Specificity of each variable was estimated. ROC curves were then generated.

### Five Year Survival as the Y-Variable

Survival in years is a numeric variable and ranged from 0.5 to 5. A basic histogram plot showed that a good proportion of patients died before year 2 (mean survival=2.45 years with standard deviation of 1.6).

The dichotomous variable “Five Year Survival” was used and assigned the value “Survive5” to all cases with a survival time (years) greater than or equal to five years. All other cases were assigned “NoSurv” or no survival. Of the total 89 complete cases, 20 survived at least five years, while 69 died before five years. Therefore the probability of

“NoSurv” was 0.775 while the probability of “Survive5” was 0.225 (based on actual outcomes).

### X Variables

The following predictor variables were analyzed: FAS (0-3 grade system); USP2a (0-3 grade system); AMACR (0-3 grade system); NPY (0-3 grade system); Gleason score (5-7 only); and Pre-therapy PSA (ng/ml). FAS, USP2a, AMACR and NPY were treated as ordinal data in the analysis with JMP version 8. Results were summarized in two stages. First, the relationship between Degrees of regression and a single predictor was analyzed. Second, the relationships between Degrees of regression and more than one predictor were analyzed. The nominal logistic regression analysis was performed and was required because the Y-variable “Survive5” is a dichotomous variable. A summary of key performance characteristics is shown in Table 27 below.

TABLE 27
Summary of Logistics Results: X variables versus Five Year Survival

| Variable | R² | p-value | ROC area | Sensitivity | Specificity |
|---|---|---|---|---|---|
| FAS | 0.567 | <.0001 | 0.937 | 0.984 | 0.704 |
| USP2a | 0.471 | <.0001 | 0.904 | 0.957 | 0.550 |
| AMACR | 0.277 | <.0001 | 0.782 | 0.985 | 0.400 |
| NPY | 0.630 | <.0001 | 0.946 | 0.969 | 0.783 |
| Gleason | 0.04 | 0.568 | 0.550 | 1 | .05 |
| (5-7) | | | | | |
| Pre-therapy | 0.008 | 0.374 | 0.589 | .986 | .000 |
| PSA | | | | | |

This table summary includes the following criteria: R² (R-squared) is a measure of the whole model. Its value is between 0 and 1. Higher values mean that the model explains more of the overall variation in the data. p-value is a measure of statistical significance. Typically in the art, a p-value below 0.05 is taken as a significant model or effect. ROC-area is a property of the ROC-Curve. A ROC-curve involves the count of true positives by false positives as one accumulates the frequencies across a rank ordering. The ROC curve plots (1-Specificity) on the X-axis and Sensitivity on the Y-axis. The largest possible are under the ROC-Curve is 1 and indicates perfect separation of true positives and true negatives. A value near 1 is desired if the variable is to be predictive.

With diagnostic tests, Sensitivity and Specificity are important concepts. Sensitivity is the probability that a given X-value (a test or measure) correctly predicts the existence of a condition. Here the X variables are detailed above and the condition would be the binary variable Survive 5 (survival for at least 5 years or not). Specificity is the probability that a test correctly predicts that a condition does not exist.

From the data, it can be determined that FAS has the second highest R², the second largest ROC area and the second highest Specificity. It has a slightly higher Sensitivity than NPY, but that is traded off with a lower Specificity. USP2a has the third highest R², the third largest ROC area and the third highest Specificity. It has a slightly lower Sensitivity than NPY. AMACR has the fourth highest R², the fourth largest ROC area and the fourth highest Specificity. It has the higher Sensitivity, but that is traded off considerably by a lower Specificity.

Interestingly, both Gleason and Pre-therapy PSA have non-significant relationships with five year survival. Their R² are low and the ROC area is near 0.5. Although their Sensitivity is 1, it is paired with a Specificity=0.5. Consequently, the logistic model classifies virtually all patients as NoSurv, because there is no significant relationship.

### Example 21

### Sensitivity and Specificity for Each X Variable: Comparison of ROC Curves

The ROC curve is a way to visualize the relationship between Sensitivity and Specificity (or 1-Specificity).

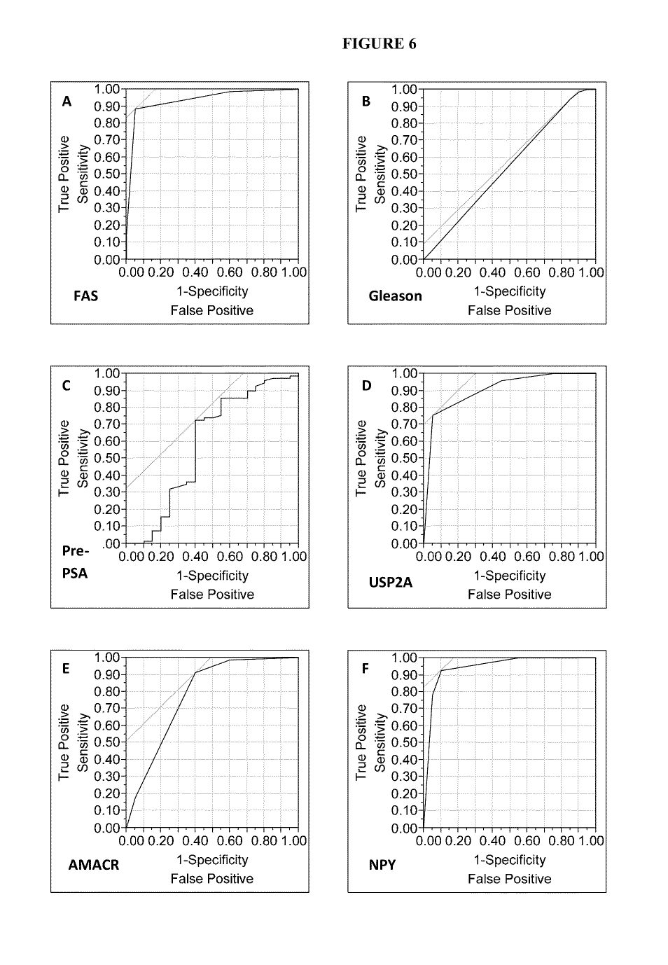

The ideal ROC curve correctly classifies all with a condition as positive and all those without the condition as negative. The area under the ideal ROC curve is 1, and that means a steep increase in sensitivity to 1 when (1-specificity)=0. ROC curves for the six X variables are shown in FIG. 6. Comparison of the steep ROC curve for FAS with the diagonal ROC from Gleason score demonstrates that Gleason is not very useful in predicting survival outcomes. Similarly, pre-therapy PSA is not a very good variable in predicting five-year survival. It is noted that the PSA ROC looks different (more like a step function), because PSA is continuous rather than ordinal like the other variables. A comparison of the ROC of the six variables under consideration clearly shows that FAS, NPY and USP2a have the largest area under the curve. Gleason and PSA have ROC curves near the diagonal and that implies that they are not very useful in predicting Five Year Survivability.

### Example 22

### Five Year Survival Probabilities Using Log-Normal Distribution

In an effort to investigate whether the four predictor variables, FAS, NPY, AMACR and USP2a can be used to obtain improved estimates of the five-year survival probabilities and how they perform compared with Pre-therapy PSA or Gleason score, a parametric survival analysis was performed. This analysis uses the log-normal distribution to represent variability and a linear model to represent the relationship between survival times and predictor variables and or Gleason/PSA. Some observations are left-censored, because there were some survival data beyond 5 years.

In this study, the Y-variable in each case is Survival Times (years). Most of the observations were rounded to whole years. A few observations are left-censored at 5 years. The censoring variable was included in the analysis. FAS, USP2a, AMACR, NPY were treated as ordinal data in the analysis with JMP.

Data for 1900 cases (male subjects) were available. Patients were post-prostatectomy with androgen ablation and no radiation therapy. Gleason scores for all were between 5-7. However, one case lacked the survival time and another case lacked some measures (AMACR and NPY). As a result, only 1888 or 1889 observations were available for analysis. Survival probabilities were estimated using only cases with Gleason score of 5, 6, and 7. Eight observations were outside this range. This reduces the number of cases further to either 1881 or 1880. It is noted, however, that including these cases (with Gleason score 4, 8, and 9) did not alter the conclusions nor change the estimate of the magnitude of the effect.

The analysis was performed using JMP 8, a product of the SAS Institute. For this study, the Fit Life Distribution was used to establish which survival distribution was most useful, and the location model of Fit Life by X platform to generate the graphs of survival probabilities versus different levels of predictor variables. Fit Parametric Survival was used for specific numeric results, such as testing the significance of each term and for the estimates of the Five Year Survival/Failure probabilities. All platforms obtain their results with likelihood methods, a widely-used and well-understood statistical methodology.

The first analysis was made to assess the survival data without reference to any predictor or other X-variable. The purpose of this analysis was to establish the most useful survival distribution. The analysis of six different common survival distributions shows that the Log-normal distribution fit the data best. Based on the Log-normal model estimates, without considering any X-variables, the five-year survival probability was 0.096.

Four predictor variables were then analyzed and each was shown to be highly significant, with FAS, USP2a and NPY as the strongest three variables. Table 28 shows the five year survival probability (Sury Prob) for cases with Gleason scores of 5-7 broken down by predictor variable grade score.

TABLE 28
5 Year Survival Probabilities

| Predictor | | L-R Chi- | Surv Prob | Surv Prob | Surv Prob | Surv Prob |
|---|---|---|---|---|---|---|
| Variable | p-value | Square | for score = 0 | for score = 1 | for score = 2 | for score = 3 |
| FAS | <0.0001 | 75.4 | .608 | .303 | .011 | .001 |
| USP2a | <0.0001 | 65.3 | .694 | .309 | .166 | .002 |
| AMACR | 0.0002 | 19.4 | .123 | .039 | .068 | .505 |
| NPY | <0.0001 | 59.8 | .694 | .315 | .020 | .005 |

The model of Survival time versus Gleason score was not significant (See Table 29). However, there was a drop in survival probability from Gleason=5 to Gleason=6. This was found to be significant when Gleason is added to other predictor variables. Nevertheless, the Gleason score model was considerably weaker both alone and in conjunction with predictor variables.

TABLE 29
Survival Probabilities-Gleason 5-7

| | | L-R Chi- | Surv Prob | Surv Prob | Surv Prob |
|---|---|---|---|---|---|
| | p-value | Square | for score = 5 | for score = 6 | for score = 7 |
| Gleason | 0.401 | 1.8 | .237 | .097 | .073 |

Likewise, pre-therapy PSA was not significant (p=0.77). The relationship of survival probability versus PSA score is inverted, i.e., as the PSA score increases, the estimated survival probability decreases.

### Table 30: Survival Probabilities-PSA

| | | | | Surv Prob | Surv Prob |
|---|---|---|---|---|---|
| | | L-R Chi- | Surv Prob | for score = | for score = |
| | p-value | Square | for score = 5 | 20 | 50 |
| PSA | 0.771 | 0.084 | .089 | .095 | 0.109 |

### Example 23

### Combinations of Predictor Variables on Survival

Utilizing the data from Example 22, combinations of predictor variables were investigated for improvements to the predictive model. First, single predictor variables were combined with Gleason scores and PSA, and then multiple predictor variables were combined with Gleason score and PSA level measurements. The models containing one predictor variable plus Gleason and PSA show very similar results. See Table 31. All single predictor variables were significant. Gleason and PSA were not significant, except when Gleason was combined with NPY. This was determined to occur primarily when NPY=0 or 1 grades. A similar relationship was observed with FAS and USP2a, yet without achieving statistical significance.

TABLE 31
Survival Probabilities-Combinations

| | Model | p- | Model | p- | Model | p- | Model | p- |
|---|---|---|---|---|---|---|---|---|
| | terms | value | terms | value | terms | value | terms | value |
| | FAS | <.0001 | USP2a | <.0001 | AMACR | 0.0001 | NPY | <.0001 |
| Adding | Gleason | 0.256 | Gleason | 0.302 | Gleason | 0.267 | Gleason | 0.028 |
| Adding | PSA | 0.937 | PSA | 0.790 | PSA | 0.925 | PSA | 0.585 |

Combinations of multiple predictor variables were also investigated and these results are shown in Tables 32-38.

TABLE 32
Survival Probabilities-Combination FAS/Gleason/PSA

| | | Pre- | | | | | |
|---|---|---|---|---|---|---|---|
| | | therapy | | | | | |
| FAS | Gleason | PSA | | Prob | Prob | Lower | Upper |
| (0-3) | Score | (ng/ml) | Time | Survival | Failure | 95% | 95% |
| 0 | 5 | 12.5 | 5 | 0.756 | 0.244 | 0.030 | 0.689 |
| 0 | 6 | 12.5 | 5 | 0.663 | 0.337 | 0.107 | 0.657 |
| 0 | 7 | 12.5 | 5 | 0.526 | 0.474 | 0.192 | 0.770 |
| 1 | 5 | 12.5 | 5 | 0.442 | 0.558 | 0.161 | 0.900 |
| 1 | 6 | 12.5 | 5 | 0.337 | 0.663 | 0.453 | 0.832 |
| 1 | 7 | 12.5 | 5 | 0.219 | 0.781 | 0.568 | 0.916 |
| 2 | 5 | 12.5 | 5 | 0.027 | 0.973 | 0.735 | 0.999 |
| 2 | 6 | 12.5 | 5 | 0.014 | 0.986 | 0.925 | 0.998 |
| 2 | 7 | 12.5 | 5 | 0.005 | 0.995 | 0.957 | 1.000 |
| 3 | 5 | 12.5 | 5 | 0.004 | 0.996 | 0.936 | 1.000 |
| 3 | 6 | 12.5 | 5 | 0.002 | 0.998 | 0.991 | 1.000 |
| 3 | 7 | 12.5 | 5 | 0.000 | 1.000 | 0.996 | 1.000 |

TABLE 33
Survival Probabilities-Combination USP2a/Gleason/PSA

| | | Pre- | | | | | |
|---|---|---|---|---|---|---|---|
| | | therapy | | | | | |
| USP2a | Gleason | PSA | | Prob | Prob | Lower | Upper |
| (0-3) | Score | (ng/ml) | Time | Survival | Failure | 95% | 95% |
| 0 | 5 | 12.5 | 5 | 0.810 | 0.190 | 0.007 | 0.757 |
| 0 | 6 | 12.5 | 5 | 0.793 | 0.207 | 0.021 | 0.656 |
| 0 | 7 | 12.5 | 5 | 0.671 | 0.329 | 0.056 | 0.760 |
| 1 | 5 | 12.5 | 5 | 0.389 | 0.611 | 0.192 | 0.924 |
| 1 | 6 | 12.5 | 5 | 0.366 | 0.634 | 0.353 | 0.856 |
| 1 | 7 | 12.5 | 5 | 0.236 | 0.764 | 0.496 | 0.926 |
| 2 | 5 | 12.5 | 5 | 0.200 | 0.800 | 0.410 | 0.972 |
| 2 | 6 | 12.5 | 5 | 0.183 | 0.817 | 0.653 | 0.921 |
| 2 | 7 | 12.5 | 5 | 0.100 | 0.900 | 0.743 | 0.972 |
| 3 | 5 | 12.5 | 5 | 0.003 | 0.997 | 0.938 | 1.000 |
| 3 | 6 | 12.5 | 5 | 0.003 | 0.997 | 0.986 | 1.000 |
| 3 | 7 | 12.5 | 5 | 0.001 | 0.999 | 0.994 | 1.000 |

TABLE 34
Survival Probabilities-Combination AMACR/Gleason/PSA

| | | Pre- | | | | | |
|---|---|---|---|---|---|---|---|
| | Glea- | therapy | | | | | |
| AMACR | son | PSA | | Prob | Prob | Lower | Upper |
| (0-3) | Score | (ng/ml) | Time | Survival | Failure | 95% | 95% |
| 0 | 5 | 12.5 | 5 | 0.334 | 0.666 | 0.237 | 0.942 |
| 0 | 6 | 12.5 | 5 | 0.104 | 0.896 | 0.707 | 0.976 |
| 0 | 7 | 12.5 | 5 | 0.092 | 0.908 | 0.705 | 0.983 |
| 1 | 5 | 12.5 | 5 | 0.163 | 0.837 | 0.476 | 0.978 |
| 1 | 6 | 12.5 | 5 | 0.035 | 0.965 | 0.913 | 0.988 |
| 1 | 7 | 12.5 | 5 | 0.030 | 0.970 | 0.915 | 0.991 |
| 2 | 5 | 12.5 | 5 | 0.256 | 0.744 | 0.290 | 0.969 |
| 2 | 6 | 12.5 | 5 | 0.068 | 0.932 | 0.796 | 0.984 |
| 2 | 7 | 12.5 | 5 | 0.060 | 0.940 | 0.800 | 0.988 |
| 3 | 5 | 12.5 | 5 | 0.807 | 0.193 | 0.016 | 0.660 |
| 3 | 6 | 12.5 | 5 | 0.514 | 0.486 | 0.217 | 0.762 |
| 3 | 7 | 12.5 | 5 | 0.487 | 0.513 | 0.224 | 0.795 |

TABLE 35
Survival Probabilities-Combination NPY/Gleason/PSA

| | | Pre- | | | | | |
|---|---|---|---|---|---|---|---|
| | | therapy | | | | | |
| NPY | Gleason | PSA | | Prob | Prob | Lower | Upper |
| (0-3) | Score | (ng/ml) | Time | Survival | Failure | 95% | 95% |
| 0 | 5 | 12.5 | 5 | 0.927 | 0.073 | 0.002 | 0.468 |
| 0 | 6 | 12.5 | 5 | 0.784 | 0.216 | 0.047 | 0.540 |
| 0 | 7 | 12.5 | 5 | 0.591 | 0.409 | 0.140 | 0.733 |
| 1 | 5 | 12.5 | 5 | 0.662 | 0.338 | 0.046 | 0.800 |
| 1 | 6 | 12.5 | 5 | 0.402 | 0.598 | 0.354 | 0.808 |
| 1 | 7 | 12.5 | 5 | 0.210 | 0.790 | 0.562 | 0.927 |
| 2 | 5 | 12.5 | 5 | 0.100 | 0.900 | 0.531 | 0.993 |
| 2 | 6 | 12.5 | 5 | 0.026 | 0.974 | 0.890 | 0.996 |
| 2 | 7 | 12.5 | 5 | 0.006 | 0.994 | 0.960 | 0.999 |
| 3 | 5 | 12.5 | 5 | 0.032 | 0.968 | 0.758 | 0.999 |
| 3 | 6 | 12.5 | 5 | 0.006 | 0.994 | 0.978 | 0.999 |
| 3 | 7 | 12.5 | 5 | 0.001 | 0.999 | 0.992 | 1.000 |

Tables 36-38 show the Effect likelihood ratio Test of the various combinations. (*) indicates statistical significance.

TABLE 36
Survival Probabilities-Combination FAS/USP2a/AMACR/Gleason/PSA

| Source | DF | L-R ChiSquare | Prob > ChiSq |
|---|---|---|---|
| NPY (0-3) | 3 | 22.6450728 | <.0001* |
| USP2a (0-3) | 3 | 12.0233808 | 0.0073* |
| AMACR (0-3) | 3 | 7.78898035 | 0.0506 |
| Gleason Score | 5 | 8.98077582 | 0.1098 |
| Pre-therapy PSA (ng/ml) | 1 | 0.02195676 | 0.8822 |

TABLE 37
Survival Probabilities-Combination NPY/USP2a/AMACR/Gleason/PSA

| Source | DF | L-R ChiSquare | Prob > ChiSq |
|---|---|---|---|
| NPY (0-3) | 3 | 22.6450728 | <.0001* |
| USP2a (0-3) | 3 | 12.0233808 | 0.0073* |
| AMACR (0-3) | 3 | 7.78898035 | 0.0506 |
| Gleason Score | 5 | 8.98077582 | 0.1098 |
| Pre-therapy PSA (ng/ml) | 1 | 0.02195676 | 0.8822 |

TABLE 38
Survival Probabilities-Combination
FAS/NPY/USP2a/AMACR/Gleason/PSA

| Source | DF | L-R ChiSquare | Prob > ChiSq |
|---|---|---|---|
| FAS (0-3) | 3 | 13.7001748 | 0.0033* |
| NPY (0-3) | 3 | 4.24880878 | 0.2358 |
| USP2a (0-3) | 3 | 9.3122571 | 0.0254* |
| AMACR (0-3) | 3 | 10.9697295 | 0.0119* |
| Gleason Score | 5 | 7.69334616 | 0.1740 |
| Pre-therapy PSA (ng/ml) | 1 | 0.10599907 | 0.7447 |

Using all variables, including both FAS and NPY, shows that NPY is not significant, because the two (NPY and FAS) are very similar. However, for these data, FAS seems to be the stronger variable.

When three of the predictor variables (FAS, USP2a and AMACR) were combined with Gleason and PSA, all three were significant, but Gleason score was not significant at alpha=0.05. See Table 39. The fact that all three were significant simultaneously suggests that they explain slightly different aspects of the trade-offs between sensitivity and specificity.

TABLE 39
Survival Probabilities

| Model terms | p-value |
|---|---|
| FAS (0-3) | <.0001 |
| USP2a (0-3) | 0.0368 |
| AMACR (0-3) | 0.0166 |
| Gleason Score | 0.0862 |
| Pre-therapy PSA (ng/ml) | 0.8483 |

### Example 24

### Failure Probabilities by Predictor Variable

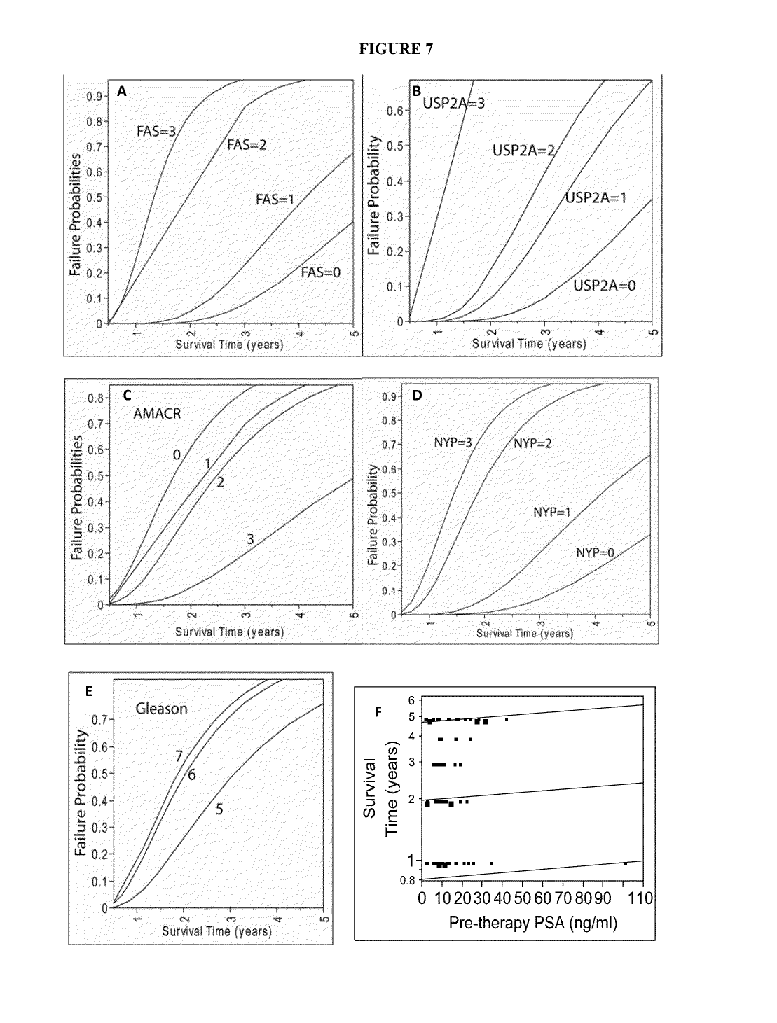

Based on a model relating survival times to each predictor variable separately, estimates of failure time (no survival) distributions by variable were calculated. These distributions can be used to explain the behavior of each predictor variable. The data are shown in FIG. 7. Each plot has Survival Time as the X-axis (from 0 to 5) and the Failure Probabilities on the Y-axis. The different distributions are labeled within each graph.

The first plot, FIG. 7A relates Survival Time to FAS. This plot shows that the curves for FAS=3 and FAS=2 are very close. Similarly, the curves of FAS=1 and FAS=0 are close. These two groups separate cases with little chance of five-year survival from those with a chance of five-year survival. The four curves are consistent, in that FAS=0 is best, FAS=3 is worst in terms of Survival. The confidence interval estimates for FAS show that the difference between FAS=1 and FAS=0 is not statistically significant, but that all other pairs of differences are significant at alpha=0.05.

The second plot, FIG. 7B, relates Survival Time to USP2a. This plot shows that the curves for USP2a=1 and USP2a=2 are very close. The only cases with a chance of five-year survival are the ones from USP2a=0. The four curves are consistent, in that USP2a=0 is best and USP2a=3 is worst in terms of Survival. The confidence interval estimates for USP2a show that the difference between USP2a=1 and USP2a=0 and the one for USP2a and USP2a=1 are not statistically significant, but that all other pairs of differences are significant at alpha=0.05.

The third plot, FIG. 7C, relates Survival Time to AMACR. This plot shows that the curves for AMACR=0, AMACR=1, and AMACR=2 are very close. The only cases with a chance of five-year survival are the ones from AMACR=3. The four curves are consistent, in that AMACR=3 is best, AMACR=0 is worst in terms of Survival. The confidence interval estimates for AMACR shows that the difference between AMACR=1 and AMACR=0 and the one for AMACR=2 and AMACR=1 are not statistically significant, but that all pairs of differences with AMACR=3 are significant at alpha=0.05.

The fourth plot, FIG. 7D, relates Survival Time to NPY. In appearance, this plot is very similar to the plot for FAS. This plot shows that the curves for NPY=3 and NPY=2 are very close. Similarly, the curves of NPY=1 and NPY=0 are close. These two groups separate cases with little chance of five-year survival from those with a chance of five-year survival. The four curves are consistent, in that NPY=0 is best, NPY=3 is worst in terms of survival. The confidence interval estimates for NPY shows that the difference between NPY=3 and NPY=2 is not statistically significant, but that all other pairs of differences are significant at alpha=0.05.

The fifth plot, FIG. 7E, relates Survival Time to Gleason Score. This plot shows that the curves for Gleason=7 and Gleason=6 are very close. The curves for Gleason=5 is not as close. The three curves are consistent, in that Gleason=5 is best, Gleason=7 is worst in terms of survival. However, the confidence interval estimates for Gleason score shows that the curves are not statistically significant at alpha=0.05.

The sixth plot, FIG. 7F, relates Survival Time to Pre-therapy PSA. This plot is different, because, unlike the other variables or Gleason, PSA is a continuous variable. The plot has Pre-therapy PSA as the X-axis and Survival Time in years as the Y-axis. The three lines represent estimates of the 0.1, 0.5 and 0.9 quantiles of Survival Time for each PSA levels. It can be seen that the resulting lines are inconsistent. As the PSA level increases, the survival probability increases. Such inconsistent results can happen when a variable is non-significant overall. This is the case with PSA with an overall p-level=0.77.

### Example 25

### Detection of FAS in Urine

The levels of FAS in urine in a series of patient samples was measured and compared to FAS levels in epithelial cells. All patients were male and each had transurethral resection (TUR). Lymph node involvement was not assessed. The data are shown in Table 40. N/A means not determined. G1 is the first Gleason score, while G2 refers to the second Gleason score and G Sum refers to the total of G1 and G2. The epithelial stain is on a scale of 0-4, where 0=0, 1=1-25%, 2=26-50%, 3=51-75% and 4=76-100%. Any measurement showing a “+” symbol refers to a level slightly above the numerical indicator. “M” stands for metastasis detected and “N” stands for nodes detected. It is evident from the table that for Gleason scores greater than 6, FAS is detected in the urine at scores of 1-4. However, at Gleason scores below 6, FAS levels tend to be low (scores 1 and 2). Therefore, there appears to be a Gleason grade threshold above which FAS spills into the urine at higher levels.

TABLE 40
Urine FAS Values

| Patient | | | | | | | | |
|---|---|---|---|---|---|---|---|---|
| Sample | | | | | G | Tumor | | |
| ID | Age | Tumor Type | G1 | G2 | Sum | Grade | Epithelium | FAS |
| A-1a | 77 | Adenocarcinoma | 3 | 3 | 6 | T1a | N | 3+ |
| A-2a | 74 | Adenocarcinoma | 3 | 2 | 5 | T1b | 1+ | 1+ |
| A-3a | 74 | Adenocarcinoma | 3 | 2 | 5 | T1b | M | 2 |
| A-4a | 62 | Adenocarcinoma | 2 | 2 | 4 | T1 | 3+ | 1+ |
| A-5a | 62 | Adenocarcinoma | 2 | 2 | 4 | T1 | 2+ | 1+ |
| A-6a | 62 | Adenocarcinoma | 2 | 2 | 4 | T1 | M | 2 |
| A-7a | 62 | Adenocarcinoma | 2 | 2 | 4 | T1 | 2+ | 1+ |
| A-8a | 62 | Adenocarcinoma | 2 | 2 | 4 | T1 | 3+ | 1+ |
| A-9a | 62 | Prostate-benign | 2 | 2 | 4 | T1 | 2+ | 1+ |
| | | hyperplasia | | | | | | |
| A-10a | 62 | Adenocarcinoma | 2 | 2 | 4 | T1 | M | 2 |
| A-1b | 85 | Adenocarcinoma | 5 | 4 | 9 | T1b | 3+ | 4 |
| A-2b | 85 | Adenocarcinoma | 5 | 4 | 9 | T1b | 4+ | 4 |
| A-3b | 85 | Adenocarcinoma | 5 | 4 | 9 | T1b | 4+ | 4 |
| A-4b | 85 | Adenocarcinoma | 5 | 4 | 9 | T1b | 3+ | 4 |
| A-5b | 85 | Adenocarcinoma | 5 | 4 | 9 | T1b | 3+ | 4 |
| A-6b | 85 | Adenocarcinoma | 5 | 4 | 9 | T1b | 3+ | 4 |
| A-7b | 85 | Adenocarcinoma | 5 | 4 | 9 | T1b | 3+ | 4 |
| A-8b | 85 | Adenocarcinoma | 5 | 4 | 9 | T1b | 3+ | 4 |
| A-9b | 85 | Adenocarcinoma | 5 | 4 | 9 | T1b | 3+ | 4 |
| A-10b | 85 | Adenocarcinoma | 5 | 4 | 9 | T1b | 3+ | 4 |
| A-1c | 85 | Adenocarcinoma | 5 | 4 | 9 | T1b | 2+ | 4 |
| A-2c | 85 | Adenocarcinoma | 5 | 4 | 9 | T1b | 3+ | 4 |
| A-3c | 85 | Adenocarcinoma | 5 | 4 | 9 | T1b | 4+ | 3 |
| A-4c | 85 | Adenocarcinoma | 5 | 4 | 9 | T1b | 4+ | 4 |
| A-5c | 85 | Adenocarcinoma | 5 | 4 | 9 | T1b | 3+ | 4 |
| A-6c | 85 | Adenocarcinoma | 5 | 4 | 9 | T1b | 2+ | 4 |
| A-7c | 85 | Adenocarcinoma | 5 | 4 | 9 | T1b | 2+ | 3 |
| A-8c | 85 | Adenocarcinoma | 5 | 4 | 9 | T1b | 3+ | 4 |
| A-9c | 85 | Adenocarcinoma | 5 | 4 | 9 | T1b | 3+ | 4 |
| A-10c | 85 | Adenocarcinoma | 5 | 4 | 9 | T1b | 2+ | 4 |
| A-1d | 78 | Adenocarcinoma | 3 | 4 | 7 | T1 | 1+ | 3 |
| A-2d | 78 | Adenocarcinoma | 3 | 4 | 7 | T1 | 2+ | 3 |
| A-3d | 78 | Adenocarcinoma | 3 | 4 | 7 | T1 | 1+ | 3 |
| A-4d | 78 | Adenocarcinoma | 3 | 4 | 7 | T1 | M | 2 |
| A-5d | 78 | Adenocarcinoma | 3 | 4 | 7 | T1 | 3+ | 3 |
| A-6d | 78 | Adenocarcinoma | 3 | 4 | 7 | T1 | 2+ | 3 |
| A-7d | 78 | Adenocarcinoma | 3 | 4 | 7 | T1 | 1+ | 2 |
| A-8d | 78 | Adenocarcinoma | 3 | 4 | 7 | T1 | 2+ | 3 |
| A-9d | 78 | Adenocarcinoma | 3 | 4 | 7 | T1 | N | 3 |
| A-10d | 78 | Adenocarcinoma | 3 | 4 | 7 | T1 | 1+ | 3 |
| A-1e | 78 | Adenocarcinoma | 3 | 4 | 7 | T1 | 2+ | 3 |
| A-2e | 78 | Adenocarcinoma | 3 | 4 | 7 | T1 | M | 3 |
| A-3e | 78 | Adenocarcinoma | 3 | 4 | 7 | T1 | 2+ | 3 |
| A-4e | 78 | Adenocarcinoma | 3 | 4 | 7 | T1 | 1+ | 2 |
| A-5e | 78 | Adenocarcinoma | 3 | 4 | 7 | T1 | 2+ | 3 |
| A-6e | 78 | Adenocarcinoma | 3 | 4 | 7 | T1 | 2+ | 3 |
| A-7e | 78 | Adenocarcinoma | 3 | 4 | 7 | T1 | 2+ | 2 |
| A-8e | 78 | Adenocarcinoma | 3 | 4 | 7 | T1 | 2+ | 3 |
| A-9e | 78 | Adenocarcinoma | 3 | 4 | 7 | T1 | 2+ | 3 |
| A-10e | 78 | Adenocarcinoma | 3 | 4 | 7 | T1 | 2+ | 4 |
| B-1a | 83 | Adenocarcinoma | 2 | 3 | 5 | T1b | 2+ | 1+ |
| B-2a | 83 | Adenocarcinoma | 2 | 3 | 5 | T1b | M | M |
| B-3a | 83 | Adenocarcinoma | 2 | 3 | 5 | T1b | 2+ | 1+ |
| B-4a | 83 | Adenocarcinoma | 2 | 3 | 5 | T1b | 2+ | 2+ |
| B-5a | 83 | Adenocarcinoma | 2 | 3 | 5 | T1b | 2+ | 1+ |
| B-6a | 83 | Adenocarcinoma | 2 | 3 | 5 | T1b | 2+ | 2+ |
| B-7a | 83 | Adenocarcinoma | 2 | 3 | 5 | T1b | 1+ | 1+ |
| B-8a | 76 | Adenocarcinoma | 3 | 5 | 8 | T1b | M | M |
| B-9a | 76 | Adenocarcinoma | 3 | 5 | 8 | T1b | 3+ | 3+ |
| B-10a | 76 | Adenocarcinoma | 3 | 5 | 8 | T1b | 3+ | 4+ |
| B-1b | 76 | Adenocarcinoma | 3 | 5 | 8 | T1b | 2+ | 3+ |
| B-2b | 76 | Adenocarcinoma | 3 | 5 | 8 | T1b | N | 3+ |
| B-3b | 76 | Adenocarcinoma | 3 | 5 | 8 | T1b | 2+ | 2+ |
| B-4b | 76 | Adenocarcinoma | 3 | 5 | 8 | T1b | 2+ | 2+ |
| B-5b | 76 | Adenocarcinoma | 3 | 5 | 8 | T1b | 2+ | 2+ |
| B-6b | 76 | Adenocarcinoma | 3 | 5 | 8 | T1b | 2+ | 3+ |
| B-7b | 76 | Adenocarcinoma | 3 | 5 | 8 | T1b | 2+ | 2+ |
| B-8b | 76 | Adenocarcinoma | 3 | 5 | 8 | T1b | M | M |
| B-9b | 76 | Adenocarcinoma | 3 | 5 | 8 | T1b | 2+ | 3+ |
| B-10b | 76 | Adenocarcinoma | 3 | 5 | 8 | T1b | 3+ | 3+ |
| B-1c | 69 | Adenocarcinoma | 5 | 5 | 10 | T1 | N | 3+ |
| B-2c | 69 | Adenocarcinoma | 5 | 5 | 10 | T1 | 2+ | 2+ |
| B-3c | 69 | Adenocarcinoma | 5 | 5 | 10 | T1 | N | 3+ |
| B-4c | 69 | Adenocarcinoma | 5 | 5 | 10 | T1 | 3+ | 2+ |
| B-5c | 69 | Adenocarcinoma | 5 | 5 | 10 | T1 | N | 3+ |
| B-6c | 69 | Adenocarcinoma | 5 | 5 | 10 | T1 | 2+ | 2+ |
| B-7c | 69 | Adenocarcinoma | 5 | 5 | 10 | T1 | 3+ | 2+ |
| B-8c | 69 | Adenocarcinoma | 5 | 5 | 10 | T1 | 3+ | 2+ |
| B-9c | 69 | Adenocarcinoma | 5 | 5 | 10 | T1 | 3+ | 2+ |
| B-10c | 69 | Adenocarcinoma | 5 | 5 | 10 | T1 | 2+ | 1+ |
| B-1d | 86 | Adenocarcinoma | 5 | 3 | 8 | T1 | 2+ | 1+ |
| B-2d | 86 | Adenocarcinoma | 5 | 3 | 8 | T1 | 2+ | 1+ |
| B-3d | 86 | Adenocarcinoma | 5 | 3 | 8 | T1 | 2+ | 1+ |
| B-4d | 86 | Adenocarcinoma | 5 | 3 | 8 | T1 | 2+ | 1+ |
| B-5d | 86 | Adenocarcinoma | 5 | 3 | 8 | T1 | 2+ | 1+ |
| B-6d | 86 | Adenocarcinoma | 5 | 3 | 8 | T1 | 2+ | 1+ |
| B-7d | 86 | Adenocarcinoma | 5 | 3 | 8 | T1 | 2+ | 1+ |
| B-8d | 86 | Adenocarcinoma | 5 | 3 | 8 | T1 | 2+ | 1+ |
| B-9d | 86 | Adenocarcinoma | 5 | 3 | 8 | T1 | 2+ | 2+ |
| B-10d | 86 | Adenocarcinoma | 5 | 3 | 8 | T1 | N | N |
| B-1e | 86 | Adenocarcinoma | 5 | 3 | 8 | T1 | 1+ | 1+ |
| B-2e | 86 | Adenocarcinoma | 5 | 3 | 8 | T1 | M | M |
| B-3e | 86 | Adenocarcinoma | 5 | 3 | 8 | T1 | 1+ | 1+ |
| B-4e | 86 | Adenocarcinoma | 5 | 3 | 8 | T1 | 1+ | 1+ |
| B-5e | 86 | Adenocarcinoma | 5 | 3 | 8 | T1 | 2+ | 2+ |
| B-6e | 86 | Adenocarcinoma | 5 | 3 | 8 | T1 | 3+ | 1+ |
| B-7e | 86 | Adenocarcinoma | 5 | 3 | 8 | T1 | 2+ | 1+ |
| B-8e | 86 | Adenocarcinoma | 5 | 3 | 8 | T1 | 2+ | 1+ |
| B-9e | 86 | Adenocarcinoma | 5 | 3 | 8 | T1 | 2+ | 1+ |
| B-10e | 86 | Adenocarcinoma | 5 | 3 | 8 | T1 | 3+ | 2+ |
| B-1f | 66 | prostatic | N/A | N/A | N/A | N/A | 2+ | 2+ |
| | | hyperplasia | | | | | | |
| B-2f | 81 | prostatic | N/A | N/A | N/A | N/A | 2+ | 2+ |
| | | hyperplasia | | | | | | |
| B-3f | 78 | prostatic | N/A | N/A | N/A | N/A | 2+ | 2+ |
| | | hyperplasia | | | | | | |
| B-4f | 78 | prostatic | N/A | N/A | N/A | N/A | 2+ | 3+ |
| | | hyperplasia | | | | | | |
| B-5f | 78 | prostatic | N/A | N/A | N/A | N/A | N | 2+ |
| | | hyperplasia | | | | | | |
| C-1a | 69 | Adenocarcinoma | 1 | 2 | 2 | T1b | 2+ | 2+ |
| C-2a | 69 | Adenocarcinoma | 1 | 2 | 2 | T1b | N | 2+ |
| C-3a | 69 | Adenocarcinoma | 1 | 2 | 2 | T1b | 2+ | 1+ |
| C-4a | 69 | Adenocarcinoma | 1 | 2 | 2 | T1b | N | 2+ |
| C-5a | 69 | Adenocarcinoma | 1 | 2 | 2 | T1b | 1+ | 2+ |
| C-6a | 69 | Adenocarcinoma | 1 | 2 | 2 | T1b | 1+ | 1+ |
| C-7a | 84 | Adenocarcinoma | 5 | 5 | 10 | T1 | 2+ | 2+ |
| C-8a | 84 | Adenocarcinoma | 5 | 5 | 10 | T1 | 2+ | 1+ |
| C-9a | 84 | Adenocarcinoma | 5 | 5 | 10 | T1 | M | M |
| C-10a | 84 | Adenocarcinoma | 5 | 5 | 10 | T1 | 2+ | 1+ |
| C-1b | 84 | Adenocarcinoma | 5 | 5 | 10 | T1 | 2+ | 1+ |
| C-2b | 84 | Adenocarcinoma | 5 | 5 | 10 | T1 | 2+ | 2+ |
| C-3b | 84 | Adenocarcinoma | 5 | 5 | 10 | T1 | 1+ | 2+ |
| C-4b | 84 | Adenocarcinoma | 5 | 5 | 10 | T1 | 2+ | 1+ |
| C-5b | 84 | Adenocarcinoma | 5 | 5 | 10 | T1 | 2+ | 1+ |
| C-6b | 84 | Adenocarcinoma | 5 | 5 | 10 | T1 | 2+ | 2+ |
| C-7b | 84 | Adenocarcinoma | 5 | 5 | 10 | T1 | 2+ | 1+ |
| C-8b | 84 | Adenocarcinoma | 5 | 5 | 10 | T1 | 1+ | 1+ |
| C-9b | 84 | Adenocarcinoma | 5 | 5 | 10 | T1 | 1+ | 1+ |
| C-10b | 84 | Adenocarcinoma | 5 | 5 | 10 | T1 | 1+ | 1+ |
| C-1c | 84 | Adenocarcinoma | 5 | 5 | 10 | T1 | 2+ | 2+ |
| C-2c | 84 | Adenocarcinoma | 5 | 5 | 10 | T1 | 2+ | 2+ |
| C-3c | 84 | Adenocarcinoma | 5 | 5 | 10 | T1 | M | M |
| C-4c | 84 | Adenocarcinoma | 5 | 5 | 10 | T1 | 2+ | 1+ |
| C-5c | 84 | Adenocarcinoma | 5 | 5 | 10 | T1 | 1+ | 1+ |
| C-6c | 84 | Adenocarcinoma | 5 | 5 | 10 | T1 | 1+ | 1+ |
| C-7c | 84 | Adenocarcinoma | 5 | 5 | 10 | T1 | 2+ | 1+ |
| C-8c | 84 | Adenocarcinoma | 5 | 5 | 10 | T1 | N | 1+ |
| C-9c | 84 | Adenocarcinoma | 5 | 5 | 10 | T1 | 2+ | 1+ |
| C-10c | 84 | Adenocarcinoma | 5 | 5 | 10 | T1 | 2+ | 1+ |
| C-1d | 84 | Adenocarcinoma | 5 | 5 | 10 | T1 | 2+ | 2+ |
| C-2d | 84 | Benign | N/A | N/A | 10 | T1 | 2+ | 2+ |
| | | prostatatic | | | | | | |
| | | hypertrophy | | | | | | |
| C-3d | 84 | Benign | N/A | N/A | 10 | T1 | 2+ | 1+ |
| | | prostatatic | | | | | | |
| | | hypertrophy | | | | | | |
| C-4d | 84 | Adenocarcinoma | 5 | 5 | 10 | T1 | 2+ | 1+ |
| C-5d | 84 | Adenocarcinoma | 5 | 5 | 10 | T1 | 1+ | 2+ |
| C-6d | 64 | Adenocarcinoma | 4 | 5 | 9 | T1 | 2+ | 2+ |
| C-7d | 64 | Adenocarcinoma | 4 | 5 | 9 | T1 | 3+ | 2+ |
| C-8d | 64 | Adenocarcinoma | 4 | 5 | 9 | T1 | 1+ | 1+ |
| C-9d | 64 | Adenocarcinoma | 4 | 5 | 9 | T1 | 3+ | 1+ |
| C-10d | 64 | Adenocarcinoma | 4 | 5 | 9 | T1 | 3+ | 1+ |
| C-1e | 64 | Adenocarcinoma | 4 | 5 | 9 | T1 | 2+ | 1+ |
| C-2e | 64 | Adenocarcinoma | 4 | 5 | 9 | T1 | N | 1+ |
| C-3e | 64 | Adenocarcinoma | 4 | 5 | 9 | T1 | 2+ | 0 |
| C-4e | 64 | Adenocarcinoma | 4 | 5 | 9 | T1 | 3+ | 0 |
| C-5e | 64 | Adenocarcinoma | 4 | 5 | 9 | T1 | 1+ | 1+ |
| C-6e | 64 | Adenocarcinoma | 4 | 5 | 9 | T1 | M | M |
| C-7e | 64 | Adenocarcinoma | 4 | 5 | 9 | T1 | 3+ | 2+ |
| C-8e | 64 | Adenocarcinoma | 4 | 5 | 9 | T1 | 2+ | 2+ |
| C-9e | 64 | Adenocarcinoma | 4 | 5 | 9 | T1 | 3+ | 2+ |
| C-10e | 64 | Adenocarcinoma | 4 | 5 | 9 | T1 | 1+ | 1+ |
| C-1f | 66 | benign prostatic | N/A | N/A | N/A | N/A | 2+ | 2+ |
| | | hpertrophy | | | | | | |
| C-2f | 66 | benign prostatic | N/A | N/A | N/A | N/A | 1+ | 1+ |
| | | hpertrophy | | | | | | |
| C-3f | 66 | benign prostatic | N/A | N/A | N/A | N/A | 2+ | 2+ |
| | | hpertrophy | | | | | | |
| D-1a | 73 | Adenocarcinoma | 2 | 3 | 5 | T1 | 3+ | 2+ |
| D-2a | 73 | Adenocarcinoma | 2 | 3 | 5 | T1 | 2+ | 2+ |
| D-3a | 73 | Adenocarcinoma | 2 | 3 | 5 | T1 | M | M |
| D-4a | 61 | Adenocarcinoma | 2 | 3 | 5 | T1 | 3+ | 2+ |
| D-5a | 61 | Adenocarcinoma | 2 | 3 | 5 | T1 | 2+ | 1+ |
| D-6a | 68 | Adenocarcinoma | 5 | 5 | 10 | T1 | 1+ | 1+ |
| D-7a | 68 | Adenocarcinoma | 5 | 5 | 10 | T1 | M | M |
| D-8a | 68 | Adenocarcinoma | 5 | 5 | 10 | T1 | 3+ | 1+ |
| D-9a | 68 | Adenocarcinoma | 5 | 5 | 10 | T1 | 3+ | 1+ |
| D-10a | 68 | Adenocarcinoma | 5 | 5 | 10 | T1 | 3+ | 2+ |
| D-1b | 68 | Adenocarcinoma | 5 | 5 | 10 | T1 | 1+ | 1+ |
| D-2b | 68 | Adenocarcinoma | 5 | 5 | 10 | T1 | 4+ | 2+ |
| D-3b | 68 | Adenocarcinoma | 5 | 5 | 10 | T1 | 1+ | 1+ |
| D-4b | 68 | Adenocarcinoma | 5 | 5 | 10 | T1 | M | M |
| D-5b | 68 | Adenocarcinoma | 5 | 5 | 10 | T1 | 1+ | 1+ |
| D-6b | 68 | Adenocarcinoma | 5 | 5 | 10 | T1 | 3+ | 1+ |
| D-7b | 68 | Adenocarcinoma | 5 | 5 | 10 | T1 | 3+ | 2+ |
| D-8b | 68 | Adenocarcinoma | 5 | 5 | 10 | T1 | 4+ | 1+ |
| D-9b | 68 | Adenocarcinoma | 5 | 5 | 10 | T1 | 4+ | 2+ |
| D-10b | 68 | Adenocarcinoma | 5 | 5 | 10 | T1 | M | M |
| D-1c | 77 | Adenocarcinoma | 5 | 5 | 10 | T1 | 1+ | 2+ |
| D-2c | 77 | Adenocarcinoma | 5 | 5 | 10 | T1 | N | 2+ |
| D-3c | 77 | Adenocarcinoma | 5 | 5 | 10 | T1 | 2+ | 1+ |
| D-4c | 77 | Adenocarcinoma | 5 | 5 | 10 | T1 | M | M |
| D-5c | 77 | Adenocarcinoma | 5 | 5 | 10 | T1 | 3+ | 1+ |
| D-6c | 77 | Adenocarcinoma | 5 | 5 | 10 | T1 | 2+ | 1+ |
| D-7c | 77 | Adenocarcinoma | 5 | 5 | 10 | T1 | 2+ | 1+ |
| D-8c | 73 | Adenocarcinoma | 2 | 4 | 6 | T1 | 3+ | 2+ |
| D-9c | 73 | Adenocarcinoma | 2 | 4 | 6 | T1 | 2+ | 1+ |
| D-10c | 73 | Adenocarcinoma | 2 | 4 | 6 | T1 | 1+ | 1+ |
| D-1d | 73 | Adenocarcinoma | 2 | 4 | 6 | T1 | 2+ | 2+ |
| D-2d | 73 | Adenocarcinoma | 2 | 4 | 6 | T1 | 3+ | 1+ |
| D-3d | 73 | Adenocarcinoma | 2 | 4 | 6 | T1 | 1+ | 1+ |
| D-4d | 73 | Adenocarcinoma | 2 | 4 | 6 | T1 | M | M |
| D-5d | 72 | Adenocarcinoma | 3 | 4 | 7 | T1 | M | M |
| D-6d | 72 | Adenocarcinoma | 3 | 4 | 7 | T1 | 3+ | 2+ |
| D-7d | 72 | Adenocarcinoma | 3 | 4 | 7 | T1 | 4+ | 2+ |
| D-8d | 94 | Adenocarcinoma | 3 | 2 | 5 | T1 | 4+ | 2+ |
| D-9d | 94 | Adenocarcinoma | 3 | 2 | 5 | T1 | 2+ | 1+ |
| D-10d | 94 | Adenocarcinoma | 3 | 2 | 5 | T1 | 2+ | 1+ |
| D-1e | 72 | Adenocarcinoma | 5 | 4 | 9 | T1 | 3+ | 2+ |
| D-2e | 72 | Adenocarcinoma | 5 | 4 | 9 | T1 | 4+ | 2+ |
| D-3e | 72 | Adenocarcinoma | 5 | 4 | 9 | T1 | 3+ | 1+ |
| D-4e | 72 | Adenocarcinoma | 5 | 4 | 9 | T1 | 3+ | 2+ |
| D-5e | 72 | Adenocarcinoma | 5 | 4 | 9 | T1 | 3+ | 2+ |
| D-6e | 72 | Adenocarcinoma | 5 | 4 | 9 | T1 | 4+ | 2+ |
| D-7e | 72 | Adenocarcinoma | 5 | 4 | 9 | T1 | 1+ | 1+ |
| D-8e | 72 | Adenocarcinoma | 5 | 4 | 9 | T1 | 2+ | 2+ |
| D-9e | 72 | Adenocarcinoma | 5 | 4 | 9 | T1 | N | 1+ |
| D-10e | 72 | Adenocarcinoma | 5 | 4 | 9 | T1 | 3+ | 2+ |
| D-1f | 65 | prostatic | N/A | N/A | N/A | N/A | 1+ | 2+ |
| | | hyperplasia | | | | | | |
| D-2f | 81 | prostatic | N/A | N/A | N/A | N/A | 1+ | 2+ |
| | | hyperplasia | | | | | | |
| D-3f | 66 | prostatic | N/A | N/A | N/A | N/A | 1+ | 1+ |
| | | hyperplasia | | | | | | |
| D-4f | 71 | benign prostatic | N/A | N/A | N/A | N/A | 1+ | 2+ |
| | | hypertrophy | | | | | | |
| D-5f | 71 | benign prostatic | N/A | N/A | N/A | N/A | 3+ | 3+ |
| | | hypertrophy | | | | | | |

Controls

| | | | | | | | | |
|---|---|---|---|---|---|---|---|---|
| 1 | 32 | normal | N/A | N/A | N/A | N/A | 2+ | N/A |
| 2 | 6 | normal | N/A | N/A | N/A | N/A | 2+ | N/A |
| 3 | 6 | normal | N/A | N/A | N/A | N/A | 2+ | N/A |
| 4 | 32 | normal | N/A | N/A | N/A | N/A | 2+ | N/A |
| 5 | 101 | normal | N/A | N/A | N/A | N/A | 1+ | N/A |
| 6 | 62 | normal | N/A | N/A | N/A | N/A | 2+ | N/A |
| 7 | 101 | normal | N/A | N/A | N/A | N/A | 3+ | N/A |
| 8 | 52 | normal | N/A | N/A | N/A | N/A | N | N/A |
| 9 | 62 | normal | N/A | N/A | N/A | N/A | 2+ | N/A |
| 10 | 32 | normal | N/A | N/A | N/A | N/A | 2+ | N/A |
| 11 | 36 | normal | N/A | N/A | N/A | N/A | N | N/A |
| 12 | 52 | normal | N/A | N/A | N/A | N/A | 4+ | N/A |

### Example 26

### Fit “X by Y Variables” With and Without Gleason Limitations

The relationship among the variables FAS, AMACR and USP2a and degree of regression were further investigated using the SAS statistical package, JMP, version 8 (Cary, N.C.). The data consisted of 90 observations which are detailed in Table 8 of Example 10. Of these data, X by Y fit was evaluated with an without a further limitation of Gleason score. In this instance, of the 90 observations, only those 82 observations with Gleason scores between 5 and 7 were used.

Two variables were used as surrogates for solid tumor (organ confined): FAS (0, 1, 2, 3) and Degree of regression. With each variable cross tabulation was performed and in the case of FAS, multivariate rank correlation using Kendall's tau and Spearman's Rho as measures of association between ordinal variables was also performed. The data for FAS, AMACR and USP2A are shown in Examples 27 and 28. The data for the evaluation across all variables including Degree of Regression are given in Example 29.

The results showed that the strongest association is between FAS and NPY with an almost equally strong association between FAS and USP2A. Regarding degree of regression, FAS was stronger than USP2A. However, with USP2A, if the cutoff was changed from USP2A=0, 1 versus 2, 3 to USP2A=0, 1, 2 versus 3, then a correlation was present.

Regarding AMACR, when FAS=3, 48 of 51 cases have AMACR=1 or 2. Thus a low AMACR value is associated with a high FAS value. However, the association is not as strong when FAS is low.

### Example 27

### Fit X by Y Variables: FAS, AMACR and USP2A

Using FAS=0 and FAS=1 as substitute for Excellent Prognosis, in Solid Tumor (organ confined) and supposing an Excellent Prognosis, the inventors sought to determine what happens to FAS, AMACR and USP2A. Twenty eight cases fell in the category. Twelve USP2A cases had values either 0 or 1, while 16 have values 2 and 3. There was a non-significant difference between the two USP2A groups. Nineteen cases have AMACR with values 0 and 1, while 9 have values. The difference between the 19 and 9 was statistically significant with p=0.044 of the exact Fisher's Test, assuming a one-sided test. The two-sided test has a Chi-square p-value=0.059 and thus missed the threshold. The contingency tables provided a fuller picture. The contingency tables are shown in Tables 41 and 42.

TABLE 41
Contingency Table FAS (0-3) By USP2A (0-3)

| | | USP2A | | | | |
|---|---|---|---|---|---|---|
| Count | | USP2A = 0 | USP2A = 1 | USP2A = 2 | USP2A = 3 | FAS Totals |
| FAS | FAS = 0 | 4 | 3 | 2 | 0 | 9 |
| | FAS = 1 | 1 | 4 | 12 | 2 | 19 |
| | FAS = 2 | 0 | 1 | 4 | 5 | 10 |
| | FAS = 3 | 0 | 1 | 5 | 46 | 52 |

| | | | | | |
|---|---|---|---|---|---|
| USP2A Totals | 5 | 9 | 23 | 53 | 90 |

TABLE 42
Contingency Table FAS (0-3) By AMACR (0-3)

| | | AMACR | | | | |
|---|---|---|---|---|---|---|
| Count | | AMACR = 0 | AMACR = 1 | AMACR = 2 | AMACR = 3 | FAS Totals |
| FAS | FAS = 0 | 5 | 0 | 0 | 4 | 9 |
| | FAS = 1 | 0 | 14 | 1 | 4 | 19 |
| | FAS = 2 | 0 | 0 | 10 | 0 | 10 |
| | FAS = 3 | 4 | 44 | 2 | 1 | 51 |

| | | | | | |
|---|---|---|---|---|---|
| AMACR Totals | 9 | 58 | 13 | 9 | 89 |

In tumors that have recurred or metastasized, the relationship between high values of FAS (3 or 4) and the values of USP2A and AMACR were also evaluated. Using FAS=2 and FAS=3 as substitute for Poor Prognosis, 61 cases fell in that category. Two USP2A cases had values either 0 or 1, while 60 had values 2 and 3. Virtually all high FAS values were associated with high USP2A values. This is a clear significant difference between the two USP2A groups.

Forty eight cases had AMACR with values 0 and 1, while 13 had values. The difference between the 48 and 13 was also statistically significant (p<0.0001). The two-sided test has a Chi-square p-value=0.059 and thus misses the threshold.

### Example 28

### Fit X by Y Variables: FAS, AMACR and USP2A

Using the “Excellent” Degree of Regression as substitute for “Excellent” Prognosis, we sought to determine what happens to FAS, AMACR and USP2A. It was found that 34 cases fell in this category with 25 FAS cases having values either 0 or 1, while 9 had values 2 and 3. This was not found to be a significant difference between the two FAS groups with p=0.006. Further, 13 USP2A cases had values either 0 or 1, while 21 had values 2 and 3. In contrast, this was a highly significant difference between the two USP2A groups. Finally, 19 cases had AMACR with values 0 and 1, while 15 had values of 2 and 3. The difference was not significant. The data are shown in Tables 43-45.

TABLE 43
Contingency Table Degree of Regression (2 grades) By FAS (0-3)

| | | FAS | | | | Degree of |
|---|---|---|---|---|---|---|
| | Count | FAS = 0 | FAS = 1 | FAS = 2 | FAS = 3 | Regression Totals |
| Degree of | Poor | 0 | 3 | 6 | 47 | 56 |
| Regression | Excellent | 9 | 16 | 4 | 5 | 34 |

| | | | | | |
|---|---|---|---|---|---|
| FAS Totals | 9 | 19 | 10 | 52 | 90 |

TABLE 44
Contingency Table Degree of Regression (2 grades) By USP2A (0-3)

| | | USP2A | | | | Degree of |
|---|---|---|---|---|---|---|
| | Count | USP2A = 0 | USP2A = 1 | USP2A = 2 | USP2A = 3 | Regression Totals |
| Degree of | Poor | 0 | 1 | 3 | 52 | 56 |
| Regression | Excellent | 5 | 8 | 20 | 1 | 34 |

| | | | | | |
|---|---|---|---|---|---|
| USP2A Totals | 5 | 9 | 23 | 53 | 90 |

TABLE 45
Contingency Table Degree of Regression (2 grades) By AMACR (0-3)

| | | AMACR | | | | Degree of |
|---|---|---|---|---|---|---|
| | Count | 0 | 1 | 2 | 3 | Regression Totals |
| Degree of | Poor | 4 | 44 | 7 | 0 | 55 |
| Regression | Excellent | 5 | 14 | 6 | 9 | 34 |

| | | | | | |
|---|---|---|---|---|---|
| AMACR Totals | 9 | 58 | 13 | 9 | 89 |

Next, for tumors that have recurred or metastasized, the same parameters were investigated, e.g., what happens to FAS, USP2A and AMACR. Using Poor Degree of Regression as substitute for Poor Prognosis, 56 cases fell in that category. By FAS results, 3 cases with FAS=1 (no FAS=0 cases), were compared with 53 cases with FAS=2 and FAS=3. The results were highly significant. In addition, one USP2A case had values either 0 or 1, while 55 had values 2 and 3. Virtually all high FAS values were associated with high USP2A values. This was a clearly significant difference between the two USP2A groups. Finally, 48 cases had AMACR with values 0 and 1, while 7 had values of 2 and 3. The difference in the between the 48 and 7 was also statistically significant (p<0.0001).

The degree of association between the variables FAS, USP2A and AMACR

Kendall's τ and Spearman's ρ (Rank Correlation) were then investigated using two measures of association between two ordinal scales. The two correlation tables (Tables 46 and 47) show that (apart from FAS and NPY) the strongest association is between FAS and USP2A, i.e., Kendall tau=0.7152 and was highly significantly different from 0 (p<0.0001). Likewise Spearman's rho=0.7697 and was highly significant.

Although associations between AMACR and FAS and USP2A are significant, the degree of association was significantly lower. The association between AMACR and FAS has a Kendall tau=−0.2452 with p=0.0098 being significantly different from 0. Likewise Spearman's rho=−0.7697 was significant with p=0.0208. The association between FAS and NPY is very high.

TABLE 46
Nonparametric: Kendall's τ

| Variable | by Variable | Kendall τ | Prob > \|τ\| |
|---|---|---|---|
| USP2A (0-3) | FAS (0-3) | 0.7152 | <.0001* |
| AMACR (0-3) | FAS (0-3) | −0.2452 | 0.0098* |
| AMACR (0-3) | USP2A (0-3) | −0.2159 | 0.0243* |
| NPY (0-3) | FAS (0-3) | 0.8104 | 0.0000* |
| NPY (0-3) | USP2A (0-3) | 0.7114 | <.0001* |
| NPY (0-3) | AMACR (0-3) | −0.1801 | 0.0583 |

TABLE 47
Nonparametric: Spearman's ρ

| Variable | by Variable | Spearman ρ | Prob > \|ρ\| |
|---|---|---|---|
| USP2A (0-3) | FAS (0-3) | 0.7697 | <.0001* |
| AMACR (0-3) | FAS (0-3) | −0.2447 | 0.0208* |
| AMACR (0-3) | USP2A (0-3) | −0.2225 | 0.0361* |
| NPY (0-3) | FAS (0-3) | 0.8506 | <.0001* |
| NPY (0-3) | USP2A (0-3) | 0.7713 | <.0001* |
| NPY (0-3) | AMACR (0-3) | −0.1872 | 0.0789 |

### Example 29

### Fit X by X Group of 90 Observations

Following the protocol outlined in Example 26, the Fit X by X data are given in the following Tables 48-54. The data are broken into 7 reporting groups A-G, below. For each group is provided a contingency table, the outline of the test R-squared values and the results of the chi squared statistical analysis. It is noted that 20% of cells had an expected count less than 5.

A. USP2 (0-3) By FAS (0-3)

TABLE 48
A. Contingency Table

| | | USP2A | | | | |
|---|---|---|---|---|---|---|
| Count | | USP2A = 0 | USP2A = 1 | USP2A = 2 | USP2A = 3 | FAS Totals |
| FAS | FAS = 0 | 4 | 3 | 2 | 0 | 9 |
| | FAS = 1 | 1 | 4 | 12 | 2 | 19 |
| | FAS = 2 | 0 | 1 | 4 | 5 | 10 |
| | FAS = 3 | 0 | 1 | 5 | 46 | 52 |

| | | | | | |
|---|---|---|---|---|---|
| USP2A Totals | 5 | 9 | 23 | 53 | 90 |

B. Test Parameters

| N | DF | −LogLike | RSquare (U) |
|---|---|---|---|
| 90 | 9 | 35.143656 | 0.3714 |

C. Chi Square

| Test | ChiSquare | Prob > ChiSq |
|---|---|---|
| Likelihood Ratio | 70.287 | <.0001* |
| Pearson | 75.802 | <.0001* |

B. AMACR (0-3) By FAS (0-3)

TABLE 49
A. Contingency Table

| | | AMACR | | | | |
|---|---|---|---|---|---|---|
| Count | | AMACR = 0 | AMACR = 1 | AMACR = 2 | AMACR = 3 | FAS Totals |
| FAS | FAS = 0 | 5 | 0 | 0 | 4 | 9 |
| | FAS = 1 | 0 | 14 | 1 | 4 | 19 |
| | FAS = 2 | 0 | 0 | 10 | 0 | 10 |
| | FAS = 3 | 4 | 44 | 2 | 1 | 51 |

| | | | | | |
|---|---|---|---|---|---|
| AMACR Totals | 9 | 58 | 13 | 9 | 89 |

B. Test Parameters

| N | DF | −LogLike | RSquare (U) |
|---|---|---|---|
| 89 | 9 | 44.366252 | 0.4871 |

C. Chi Square

| Test | ChiSquare | Prob > ChiSq |
|---|---|---|
| Likelihood Ratio | 88.733 | <.0001* |
| Pearson | 111.112 | <.0001* |

C. NPY (0-3) By FAS (0-3)

TABLE 50
A. Contingency Table

| | | NPY | | | | |
|---|---|---|---|---|---|---|
| | | | | | NPY = | NPY |
| | Count | NPY = 0 | NPY = 1 | NPY = 2 | 3 | Totals |
| FAS | FAS = 0 | 8 | 0 | 1 | 0 | 9 |
| | FAS = 1 | 1 | 15 | 1 | 2 | 19 |
| | FAS = 2 | 0 | 0 | 5 | 5 | 10 |
| | FAS = 3 | 0 | 0 | 4 | 47 | 51 |

| | | | | | |
|---|---|---|---|---|---|
| NPY Totals | 9 | 15 | 11 | 54 | 89 |

B. Test Parameters

| N | DF | −LogLike | RSquare (U) |
|---|---|---|---|
| 89 | 9 | 59.281630 | 0.6092 |

C. Chi Square

| Test | ChiSquare | Prob > ChiSq |
|---|---|---|
| Likelihood Ratio | 118.563 | <.0001* |
| Pearson | 152.050 | <.0001* |

D. FAS (0-3) By Degree of Regression (2 Grades)

TABLE 51
A. Contingency Table

| Count | 0 | 1 | 2 | 3 | |
|---|---|---|---|---|---|
| Poor | 0 | 3 | 6 | 47 | 56 |
| Excellent | 9 | 16 | 4 | 5 | 34 |
| | 9 | 19 | 10 | 52 | 90 |

B. Test Parameters

| N | DF | −LogLike | RSquare (U) |
|---|---|---|---|
| 90 | 3 | 28.189170 | 0.2797 |

C. Chi Square

| Test | ChiSquare | Prob > ChiSq |
|---|---|---|
| Likelihood Ratio | 56.378 | <.0001* |
| Pearson | 49.817 | <.0001* |

E. USP2A (0-3) By Degree of Regression (2 Grades)

TABLE 52
A. Contingency Table

| Count | 0 | 1 | 2 | 3 | |
|---|---|---|---|---|---|
| Poor | 0 | 1 | 3 | 52 | 56 |
| Excellent | 5 | 8 | 20 | 1 | 34 |
| | 5 | 9 | 23 | 53 | 90 |

B. Test Parameters

| N | DF | −LogLike | RSquare (U) |
|---|---|---|---|
| 90 | 3 | 42.660746 | 0.4509 |

C. Chi Square

| Test | ChiSquare | Prob > ChiSq |
|---|---|---|
| Likelihood Ratio | 85.321 | <.0001* |
| Pearson | 70.947 | <.0001* |

F. AMACR (0-3) By Degree of Regression (2 Grades)

TABLE 53
A. Contingency Table

| Count | 0 | 1 | 2 | 3 | |
|---|---|---|---|---|---|
| Poor | 4 | 44 | 7 | 0 | 55 |
| Excellent | 5 | 14 | 6 | 9 | 34 |
| | 9 | 58 | 13 | 9 | 89 |

B. Test Parameters

| N | DF | −LogLike | RSquare (U) |
|---|---|---|---|
| 89 | 3 | 11.979438 | 0.1315 |

C. Chi Square

| Test | ChiSquare | Prob > ChiSq |
|---|---|---|
| Likelihood Ratio | 23.959 | <.0001* |
| Pearson | 20.915 | 0.0001* |

G. NPY (0-3) By Degree of Regression (2 Grades)

TABLE 54
A. Contingency Table

| Count | 0 | 1 | 2 | 3 | |
|---|---|---|---|---|---|
| Poor | 0 | 1 | 4 | 50 | 55 |
| Excellent | 9 | 14 | 7 | 4 | 34 |
| | 9 | 15 | 11 | 54 | 89 |

B. Test Parameters

| N | DF | −LogLike | RSquare (U) |
|---|---|---|---|
| 89 | 3 | 34.045993 | 0.3499 |

C. Chi Square

| Test | ChiSquare | Prob > ChiSq |
|---|---|---|
| Likelihood Ratio | 68.092 | <.0001* |
| Pearson | 58.576 | <.0001* |

### Example 30

### Multivariate and Univariate Analysis of all 90 Observations

With each variable, cross tabulation was performed for both multivariate and univariate rank correlation using Kendall's tau and Spearman's Rho as measures of association between ordinal variables was also performed. The analyses are shown Tables 55-58. The correlations are estimated by REML method. It should be noted that statistics were calculated for each column independently without regard for missing values in other columns.

TABLE 55
Correlations

| | FAS (0-3) | USP2A (0-3) | AMACR (0-3) | NPY (0-3) |
|---|---|---|---|---|
| FAS (0-3) | 1.0000 | 0.7685 | −0.2494 | 0.8897 |
| USP2A (0-3) | 0.7685 | 1.0000 | −0.2021 | 0.7627 |
| AMACR (0-3) | −0.2494 | −0.2021 | 1.0000 | −0.2227 |
| NPY (0-3) | 0.8897 | 0.7627 | −0.2227 | 1.0000 |

TABLE 56
Nonparametric: Kendall's τ

| Variable | by Variable | Kendall τ | Prob > \|τ\| |
|---|---|---|---|
| USP2A (0-3) | FAS (0-3) | 0.7152 | <.0001* |
| AMACR (0-3) | FAS (0-3) | −0.2452 | 0.0098* |
| AMACR (0-3) | USP2A (0-3) | −0.2159 | 0.0243* |
| NPY (0-3) | FAS (0-3) | 0.8104 | 0.0000* |
| NPY (0-3) | USP2A (0-3) | 0.7114 | <.0001* |
| NPY (0-3) | AMACR (0-3) | −0.1801 | 0.0583 |

TABLE 57
Nonparametric: Spearman's ρ

| Variable | by Variable | Spearman ρ | Prob > \|ρ\| |
|---|---|---|---|
| USP2A (0-3) | FAS (0-3) | 0.7697 | <.0001* |
| AMACR (0-3) | FAS (0-3) | −0.2447 | 0.0208* |
| AMACR (0-3) | USP2A (0-3) | −0.2225 | 0.0361* |
| NPY (0-3) | FAS (0-3) | 0.8506 | <.0001* |
| NPY (0-3) | USP2A (0-3) | 0.7713 | <.0001* |
| NPY (0-3) | AMACR (0-3) | −0.1872 | 0.0789 |

TABLE 58
Univariate Simple Statistics

| Column | N | DF | Mean | Std Dev | Minimum | Maximum |
|---|---|---|---|---|---|---|
| FAS (0-3) | 90 | 89.00 | 2.1667 | 1.0836 | 0.0000 | 3.0000 |
| USP2A (0-3) | 90 | 89.00 | 2.3778 | 0.8815 | 0.0000 | 3.0000 |
| AMACR (0-3) | 89 | 88.00 | 1.2472 | 0.7728 | 0.0000 | 3.0000 |
| NPY (0-3) | 89 | 88.00 | 2.2360 | 1.0662 | 0.0000 | 3.0000 |

### Example 31

### Fit Y by X Group; 82 Observations With Gleason Between 5 and 7

Following the protocol outlined in Example 26 with the subgroup of observations showing a Gleason score of between 5-7, the Fit X by X data are given in the following Tables 59-61. The data are broken into 3 reporting groups A-C, below. For each group is provided a contingency table, the outline of the test R-squared values and the results of the chi squared statistical analysis. It is noted that 20% of cells had an expected count less than 5.

A. USP2A (0-3) By FAS (0-3)

TABLE 59
A. Contingency Table

| | | USP2A | | | | |
|---|---|---|---|---|---|---|
| Count | | USP2A = 0 | USP2A = 1 | USP2A = 2 | USP2A = 3 | Row Total |
| FAS | FAS = 0 | 4 | 3 | 1 | 0 | 8 |
| | FAS = 1 | 0 | 4 | 11 | 2 | 17 |
| | FAS = 2 | 0 | 1 | 4 | 4 | 9 |
| | FAS = 3 | 0 | 1 | 4 | 43 | 48 |

| | | | | | |
|---|---|---|---|---|---|
| Column Total | 4 | 9 | 20 | 49 | 82 |

B. Test Parameters

| N | DF | −LogLike | RSquare (U) |
|---|---|---|---|
| 82 | 9 | 35.540582 | 0.4161 |

Square Root of Correlation = 0.645

C. Chi Square

| Test | ChiSquare | Prob > ChiSq |
|---|---|---|
| Likelihood Ratio | 71.081 | <.0001* |
| Pearson | 85.210 | <.0001* |

B. AMACR (0-3) By FAS (0-3)

TABLE 60
A. Contingency Table

| | | AMACR | | | | |
|---|---|---|---|---|---|---|
| Count | | AMACR = 0 | AMACR = 1 | AMACR = 2 | AMACR = 3 | FAS Totals |
| FAS | FAS = 0 | 5 | 0 | 0 | 3 | 8 |
| | FAS = 1 | 0 | 13 | 0 | 4 | 17 |
| | FAS = 2 | 0 | 0 | 9 | 0 | 9 |
| | FAS = 3 | 4 | 40 | 2 | 1 | 47 |

| | | | | | |
|---|---|---|---|---|---|
| AMACR Totals | 9 | 53 | 11 | 8 | 81 |

B. Test Parameters

| N | DF | −LogLike | RSquare (U) |
|---|---|---|---|
| 81 | 9 | 41.699608 | 0.5040 |

C. Chi Square

| Test | ChiSquare | Prob > ChiSq |
|---|---|---|
| Likelihood Ratio | 83.399 | <.0001* |
| Pearson | 105.444 | <.0001* |

C. NPY (0-3) By FAS (0-3)

TABLE 61
A. Contingency Table

| | | NPY | | | | |
|---|---|---|---|---|---|---|
| | | | | | NPY = | FAS |
| Count | | NPY = 0 | NPY = 1 | NPY = 2 | 3 | Totals |
| FAS | FAS = 0 | 7 | 0 | 1 | 0 | 8 |
| | FAS = 1 | 1 | 13 | 1 | 2 | 17 |
| | FAS = 2 | 0 | 0 | 5 | 4 | 9 |
| | FAS = 3 | 0 | 0 | 4 | 43 | 47 |

| | | | | | |
|---|---|---|---|---|---|
| NPY Totals | 8 | 13 | 11 | 49 | 81 |

B. Test Parameters

| N | DF | −LogLike | RSquare (U) |
|---|---|---|---|
| 81 | 9 | 52.583504 | 0.5915 |

C. Chi Square

| Test | ChiSquare | Prob > ChiSq |
|---|---|---|
| Likelihood Ratio | 105.167 | <.0001* |
| Pearson | 136.227 | <.0001* |

### Example 32

### By Degree of Regression (2 Grades); Fit Y by X Group for Cases With Gleason Between 5 and 7

Following the protocol outlined in Example 26 with the subgroup of observations showing a Gleason score of between 5-7 and by Degree of Regression, the Fit X by X data are given in the following Tables 62-65. The data are broken into 4 reporting groups A-D, below. For each group is provided a contingency table, the outline of the test R-squared values and the results of the chi squared statistical analysis. It is noted that 20% of cells had an expected count less than 5.

A. FAS (0-3) by Degree of Regression (2 Grades)

TABLE 62
A. Contingency Table

FAS

| Count (FAS) | 0 | 1 | 2 | 3 | |
|---|---|---|---|---|---|
| Poor | 0 | 3 | 5 | 43 | 51 |
| Excellent | 8 | 14 | 4 | 5 | 31 |
| | 8 | 17 | 9 | 48 | 82 |

B. Test Parameters

| N | DF | −LogLike | RSquare (U) |
|---|---|---|---|
| 82 | 3 | 24.230772 | 0.2664 |

C. Chi Square

| Test | ChiSquare | Prob > ChiSq |
|---|---|---|
| Likelihood Ratio | 48.462 | <.0001* |
| Pearson | 42.992 | <.0001* |

B. USP2A (0-3) by Degree of Regression (2 Grades)

TABLE 63
A. Contingency Table

| | |
|---|---|
| Count | USP2A |

| (USP2A) | 0 | 1 | 2 | 3 | |
|---|---|---|---|---|---|
| Poor | 0 | 1 | 2 | 48 | 51 |
| Excellent | 4 | 8 | 18 | 1 | 31 |
| | 4 | 9 | 20 | 49 | 82 |

B. Test Parameters

| N | DF | −LogLike | RSquare (U) |
|---|---|---|---|
| 82 | 3 | 39.851573 | 0.4666 |

C. Chi Square

| Test | ChiSquare | Prob > ChiSq |
|---|---|---|
| Likelihood Ratio | 79.703 | <.0001* |
| Pearson | 66.398 | <.0001* |

C. AMACR (0-3) by Degree of Regression (2 Grades)

TABLE 64
A. Contingency Table

| | |
|---|---|
| Count | AMACR |

| (AMACR) | 0 | 1 | 2 | 3 | |
|---|---|---|---|---|---|
| Poor | 4 | 40 | 6 | 0 | 50 |
| Excellent | 5 | 13 | 5 | 8 | 31 |
| | 9 | 53 | 11 | 8 | 81 |

B. Test Parameters

| N | DF | −LogLike | RSquare (U) |
|---|---|---|---|
| 81 | 3 | 10.607920 | 0.1282 |

C. Chi Square

| Test | ChiSquare | Prob > ChiSq |
|---|---|---|
| Likelihood Ratio | 21.216 | <.0001* |
| Pearson | 18.519 | 0.0003* |

D. NPY (0-3) by Degree of Regression (2 Grades)

TABLE 65
A. Contingency Table

NPY

| Count (NPY) | 0 | 1 | 2 | 3 | |
|---|---|---|---|---|---|
| Poor | 0 | 1 | 4 | 45 | 50 |
| Excellent | 8 | 12 | 7 | 4 | 31 |
| | 8 | 13 | 11 | 49 | 81 |

B. Test Parameters

| N | DF | −LogLike | RSquare (U) |
|---|---|---|---|
| 81 | 3 | 29.305661 | 0.3297 |

C. Chi Square

| Test | ChiSquare | Prob > ChiSq |
|---|---|---|
| Likelihood Ratio | 58.611 | <.0001* |
| Pearson | 50.769 | <.0001* |

### Example 33

### Multivariate and Univariate Observations With Gleason From 5 to 7

With each variable, cross tabulation was performed for both multivariate and univariate rank correlation using Kendall's tau and Spearman's Rho as measures of association between ordinal variables was also performed. The analyses are shown Tables 66-69. The correlations are estimated by REML method. It should be noted that statistics were calculated for each column independently without regard for missing values in other columns.

TABLE 66
Correlations

| | FAS (0-3) | USP2A (0-3) | AMACR (0-3) | NPY (0-3) |
|---|---|---|---|---|
| FAS (0-3) | 1.0000 | 0.7899 | −0.1953 | 0.8819 |
| USP2A (0-3) | 0.7899 | 1.0000 | −0.2189 | 0.7775 |
| AMACR (0-3) | −0.1953 | −0.2189 | 1.0000 | −0.1745 |
| NPY (0-3) | 0.8819 | 0.7775 | −0.1745 | 1.0000 |

TABLE 67
Univariate Simple Statistics

| Column | N | DF | Mean | Std Dev | Minimum | Maximum |
|---|---|---|---|---|---|---|
| FAS (0-3) | 82 | 81.00 | 2.1829 | 1.0787 | 0.0000 | 3.0000 |
| USP2A (0-3) | 82 | 81.00 | 2.3902 | 0.8714 | 0.0000 | 3.0000 |
| AMACR (0-3) | 81 | 80.00 | 1.2222 | 0.7746 | 0.0000 | 3.0000 |
| NPY (0-3) | 81 | 80.00 | 2.2469 | 1.0551 | 0.0000 | 3.0000 |

TABLE 68
Nonparametric: Kendall's τ

| Variable | by Variable | Kendall τ | Prob > \|τ\| |
|---|---|---|---|
| USP2A (0-3) | FAS (0-3) | 0.7339 | <.0001* |
| AMACR (0-3) | FAS (0-3) | −0.1959 | 0.0493* |
| AMACR (0-3) | USP2A (0-3) | −0.2132 | 0.0340* |
| NPY (0-3) | FAS (0-3) | 0.8033 | <.0001* |
| NPY (0-3) | USP2A (0-3) | 0.7164 | <.0001* |
| NPY (0-3) | AMACR (0-3) | −0.1450 | 0.1460 |

TABLE 69
Nonparametric: Spearman's ρ

| Variable | by Variable | Spearman ρ | Prob > \|ρ\| |
|---|---|---|---|
| USP2A (0-3) | FAS (0-3) | 0.7819 | <.0001* |
| AMACR (0-3) | FAS (0-3) | −0.1905 | 0.0885 |
| AMACR (0-3) | USP2A (0-3) | −0.2185 | 0.0501 |
| NPY (0-3) | FAS (0-3) | 0.8433 | <.0001* |
| NPY (0-3) | USP2A (0-3) | 0.7713 | <.0001* |
| NPY (0-3) | AMACR (0-3) | −0.1475 | 0.1888 |

1. A method for the binary stratification of a subject suspected of having prostate cancer comprising;
determining the level or grade of one or more predictor variables selected from the group consisting of FAS, NPY, USP2a, and AMACR in a samples obtained from the subject; and stratifying the subject as likely to survive at least 5 years or not based on the level or grade of said one or more predictor variables, wherein the grade of the predictor variable is less than 3.

2. The method of claim 1 wherein the subject has previously been screened for elevated prostate specific antigen (PSA).

3. The method of claim 2 wherein the predictor variable is USP2a.

4. The method of claim 2 wherein the predictor variable is AMACR.

5. The method of claim 1 wherein the grade of the predictor variable is less than 2.

6. The method of claim 5 wherein the predictor variable is FAS.

7. The method of claim 5 wherein the predictor variable is NPY.

8. The method of claim 2 wherein the measurement of PSA is total PSA.

9. The method of claim 2, wherein the subject has undergone a tissue biopsy and wherein evaluation of the biopsy revealed a tissue Gleason score of between 5 and 7.

10. The method of claim 1 wherein determining the level or grade of one or more predictor variables comprises measuring either the RNA or protein levels of said one or more predictor variables.

11. The method of claim 10 wherein protein levels are measured.

12. The method of claim 11 wherein protein levels are measured by an immunohistochemical assay.

13. The method of claim 12 wherein the immunohistochemical assay utilizes one or more predictor variable specific antibodies.

14. The method of claim 13, wherein said one or more predictor variable specific antibodies contains a detectable label.

15. An immunohistochemical kit or assay for the prediction of 5-year survival in a subject
comprising one or more predictor variable specific antibodies, each comprising a detectable label and selected from the group consisting of FAS, NPY, USP2a, and AMACR.

16. The immunohistochemical kit or assay of claim 15, further comprising a probe targeting the prostate specific antigen (PSA) gene or protein.
